(19) United States
(12) Patent Application Publication
(10) Pub. No.: US 2024/0033200 A1
(43) Pub. Date: Feb. 1, 2024

(54) SEMI-PERMANENT TATTOOS

(71) Applicant: Inkbox Ink Inc., Toronto (CA)

(72) Inventors: Christopher B. CAPUTO, Toronto (CA); Sanjay MANHAS, Toronto (CA); Ian MALLOV, Halifax (CA); Ingrid UM NLEND, Toronto (CA); Fiona JEEVA, Toronto (CA); Tyler J. HANDLEY, Toronto (CA); Charley Nicole GARRARD, North York (CA)

(73) Assignee: Inkbox Ink Inc., Toronto, ON (CA)

(21) Appl. No.: 18/041,339

(22) PCT Filed: Aug. 12, 2021
(86) PCT No.: PCT/US2021/045767

§ 371 Date: Feb. 10, 2023

Related U.S. Application Data
(60) Provisional application No. 63/064,885, filed on Aug. 12, 2020.

(51) Int. Cl. A61K 8/49; A61K 8/35; A61K 8/60; A61K 8/73; A61K 8/81; C07D 311/94; C07F 7/18; A61Q 1/02

(52) U.S. Cl. CPC A61K 8/498; A61K 8/355; A61K 8/60; A61K 8/731; A61K 8/8147; C07D 311/94; C07F 7/1804; A61Q 1/025; A61K 2800/95; A61K 2800/432

(57) ABSTRACT

The present disclosure relates to semi-permanent tattoos, for example, compositions comprising semi-permanent colorants and semi-permanent colorant precursors that include derivatives of genipin and lawsone. In some embodiments, the composition comprises: a semi-permanent colorant present in an amount of about 10% to about 20% w/w of the composition; a solvent present in an amount of about 65% to about 80% w/w of the composition; and a thickening agent present in an amount of 0.01% to about 0.3% w/w of the composition. In some embodiments, the composition further includes one or more of a film-forming agent, a humectant, and a preservative.

### CROSS REFERENCE TO RELATED APPLICATIONS

This application is a National Stage Application of International Application PCT/US2021/045767, filed on Aug. 12, 2021, now published as WO 2022/036113 A1 and which claims priority to U.S. Provisional Patent Application Ser. No. 63/064,885, filed on Aug. 12, 2020, the entire contents of which are hereby incorporated by reference.

### TECHNICAL FIELD

This disclosure relates to semi-permanent tattoos, and more particularly to compositions comprising a genipin derivative, lawsone, a lawsone derivative, or combinations thereof as well as layered adhesive articles and methods for application of compositions comprising a genipin derivative, lawsone, a lawsone derivative, or combinations thereof.

### BACKGROUND

Temporary or semi-permanent tattoo or body inks have been used throughout human history to decorate the body. Generally, temporary or semi-permanent tattoos are transferred to the skin through the direct exposure of the skin to tattoo ink over a designated incubation period. The quality of the transferred image and its duration on the skin can depend on the ink distribution profile in the outer layer of the skin called the stratum corneum. Current manufacturing processes for pre-fabricated tattoo designs include the utilization of flexographic or gravure printing, stencils, and inkjet printers. However, due to the barrier properties of the stratum corneum, many of the current methods require moderate oversight by the end user during the tattoo application process to ensure adequate ink delivery to the skin such that the tattoo ink develops properly.

### SUMMARY

Provided herein are compositions comprising: a semi-permanent colorant, wherein the semi-permanent colorant comprises a genipin derivative, lawsone, a lawsone derivative, or combinations thereof; a solvent; and a semi-volatile semi-permanent colorant solubilizer.

Also provided herein are compositions comprising: a semi-permanent colorant, wherein the semi-permanent colorant comprises a genipin derivative, lawsone, a lawsone derivative, or combinations thereof; a solvent; and a thickening agent.

Also provided herein are compositions comprising: a semi-permanent colorant, wherein the semi-permanent colorant comprises a genipin derivative, lawsone, a lawsone derivative, or combinations thereof; a solvent; and a film-forming agent.

In some embodiments, the semi-permanent colorant further comprises genipin. In some embodiments, the genipin derivative is a compound of Formula I:

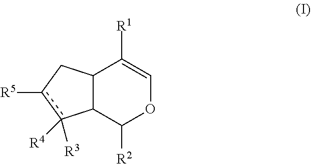

wherein:
R¹ is —C(═O)OR⁶ or phenyl substituted with 1-5 R⁷;
R⁶ is H, C₁₋₆ alkyl, or phenyl substituted with 1-5 R⁷;
each R⁷ is independently an electron withdrawing or an electron donating group;
R² is H, —OR⁹, or OC(═O)R⁹;
R⁹ is H or C₁₋₆ alkyl; and
the dotted line indicates the optional presence of a double bond;
   wherein if the double bond is absent, then R⁴ and R⁵ together with the carbon atoms to which they are attached form a 3-4 membered heterocyclic ring and R³ is —OH or —CH₂OH; or
   if the double bond is present, then R⁴ and R⁵ are absent and R³ is selected from the group consisting of: —NR⁷R⁸, —CH₂NR⁷R⁸, —C(═O)OR¹⁰, —CH(═O), phenyl, —CHR¹⁰R¹⁰, —CH═CHR¹⁰, —CH₂OR¹⁰, diethyl malonate, and phenyl substituted with 1-5 R⁷;
   wherein R¹⁰ is selected from H, C₁₋₆ alkyl, alkoxy, oxo, —OH, —CH(═O) and phenyl substituted with 1-5 R⁷; and
R⁸ is H or W.

In some embodiments, R⁶ methyl.

In some embodiments, R¹ is —C(═O)OR⁶ and R⁶ is methyl.

In some embodiments, R¹ is —C(═O)OR⁶ and R⁶ is phenyl substituted with 1-5 R⁷.

In some embodiments, R² is —OC(═O)CH₃.

In some embodiments, R² is —OH.

In some embodiments, the double bond is absent.

In some embodiments, R⁴ and R⁵ together with the carbon atoms to which they are attached form a 3-membered heterocyclic ring and R³ is —CH₂OH. In some embodiments, the 3-membered heterocyclic ring is:

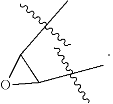 [SMILES: *C1OC1*]

In some embodiments, R⁴ and R⁵ together with the carbon atoms to which they are attached form a 4-membered heterocyclic ring and R³ is —OH. In some embodiments, the 4-membered ring is:

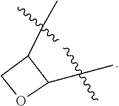 [SMILES: *C1COC1*]

In some embodiments, the double bond is present.

In some embodiments, R¹ is phenyl substituted with 1-3 R⁷.

In some embodiments, R¹ is —C(═O)OR⁶ and R⁶ is phenyl substituted with 1-3 R⁷.

In some embodiments, R² is —OH and R³ is CH₂OH.

In some embodiments, R³ is selected from the group consisting of: —NR⁷R⁸, —CH₂—NR⁷R⁸, —C(═O)OR¹⁰, —CH(═O), phenyl, —CHR¹⁰R¹⁰, —CH═CHR¹⁰, —CH₂OR¹⁰, diethyl malonate, and phenyl substituted with 1-5 R⁷; R¹ is —C(═O)OH; and R² is —OH.

In some embodiments, the genipin derivative is a compound selected from the group consisting of the compounds delineated in Table 1.

In some embodiments, R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO, —NO₂, —SO₂R⁸, —CN, —CR¹¹, —COR¹¹, —CHO, —CO₂R⁸, —C(═O)NR⁸R⁸, and halide; wherein R⁸ is selected from the group consisting of: H, —(C₁₋₉ alkyl), and —(C₂₋₉ alkenyl); and R¹¹ is halide.

In some embodiments, the lawsone derivative is the compound delineated in Table 4.

In some embodiments, the lawsone derivative is a compound of Formula III:

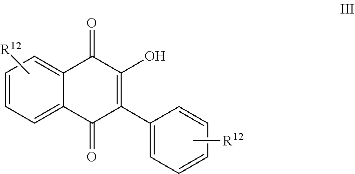

wherein R¹² is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO, —NO₂, —SO₂R⁸, —CN, —CR¹¹₃, —COR¹¹, —CHO, —CO₂R⁸, —C(═O)NR⁸R⁸, and halide;
wherein R⁸ is selected from the group consisting of: H, —(C₁₋₉ alkyl), and —(C₂₋₉ alkenyl); and R¹¹ is halide.

In some embodiments, the semi-permanent colorant is a combination of two or more of: genipin, a genipin derivative, lawsone, and a lawsone derivative.

In some embodiments, the semi-permanent colorant is a combination of genipin and Compound 1. In some embodiments, the ratio of genipin to Compound 1 is about 4:1 to about 1:4 genipin:Compound 1. In some embodiments, the ratio of genipin to Compound 1 is about 1:1 genipin:Compound 1. In some embodiments, semi-permanent colorant is a combination of Compound 1 and Compound 14. In some embodiments, the ratio of Compound 1 to Compound 14 is about 4:1 to about 1:4 Compound 1:Compound 14. In some embodiments, the ratio of Compound 1 to Compound 14 is about 1:1 Compound 1:Compound 14. In some embodiments, the semi-permanent colorant is a combination of genipin and Compound 14. In some embodiments, the ratio of genipin to Compound 14 is about 4:1 to about 1:4 genipin:Compound 14. In some embodiments, the ratio of genipin to Compound 14 is about 1:1 genipin:Compound 14. In some embodiments, the semi-permanent colorant is a combination of genipin, Compound 1, and Compound 14. In some embodiments, the ratio of genipin to Compound 1 to Compound 14 is about 4:1:1 to about 1:1:4 genipin:Compound 1:Compound 14. In some embodiments, the ratio of genipin to Compound 1 to Compound 14 is about 1:1:1 genipin:Compound 1:Compound 14.

Also provided herein are composition comprises a semi-permanent colorant, wherein the semi-permanent colorant comprises a genipin derivative, lawsone, a lawsone derivative, or combinations thereof and a thickening agent.

Also provided herein are compounds of Formula IIb:

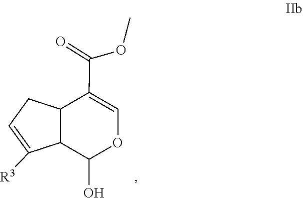

or a pharmaceutically acceptable salt thereof. In some embodiments:
R³ is selected from the group consisting of:—CH₂NR⁷R⁸, —C(═O)OR¹⁰, —CH(═O), —CHR¹⁰R¹⁰, —CH═CHR¹⁰, —CH₂OR¹⁰, and phenyl substituted with 1-5 R⁷;
each R¹⁰ is independently selected from H, —OH, —CH(═O) and phenyl substituted with 1-5 R⁷.

Also provided herein are compounds of Formula IIb:

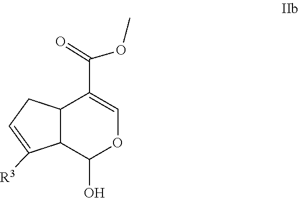

wherein:
R³ is selected from the group consisting of:—CH₂NR⁷R⁸, —C(═O)OR¹⁰, —CH(═O), —CHR¹⁰R¹⁰, —CH═CHR¹⁰, —CH₂OR¹⁰, and phenyl substituted with 1-5 R⁷;
wherein each R¹⁰ is independently selected from H, —OH, —CH(═O) and phenyl substituted with 1-5 R⁷; and
R⁷ is an electron withdrawing or an electron donating group.

In some embodiments, the compound is selected from the group consisting of the compounds delineated in Table 3. In some embodiments, the compound is selected from the group consisting of the compounds delineated in Table 2 or Table 3.

In some embodiments, R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, halide, and —NO; wherein R⁸ is selected from the group consisting of: H, —(C₁₋₉ alkyl), —(C₂₋₉ alkenyl), unsubstituted —(C₂₋₉ alkynyl); and R⁹ is halide.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

As used herein, a “colorant” refers to a substance that changes the color of reflected or transmitted light as the result of wavelength-selective absorption.

As used herein, a “temporary colorant” refers to a colorant that sits on top of the skin or, if it penetrates the skin, can diffuse out of the skin or can be washed off by, for example, water, soap, and/or isopropanol.

As used herein, “color” refers to wavelengths of electromagnetic radiation visible to the human eye.

“Surface-active agent” or “surfactant” as used herein refers to a substance that tends to reduce the surface tension of a liquid in which it is dissolved.

A “surface” as described herein refers to a portion of a layer of material (e.g., backing, adhesive backing, an adhesive layer, etc.) that may be substantially parallel to a different layer. In some embodiments, the surface area of a surface of a particular layer described herein may be larger than the surface of an edge of the particular layer. For example, an edge of a particular layer can be substantially perpendicular to a surface of the particular layer. As used herein, the term “substantially” intends that the characteristic needs not be absolute, but is close enough so as to achieve the advantages of the characteristic. For example, “substantially parallel” is not limited to absolute parallelism, and can include orientations that are intended to be parallel but due to manufacturing limitations may not be precisely parallel. For example, “substantially parallel” features are at least closer to a parallel orientation than a perpendicular orientation, and generally are formed within a few degrees of parallel. Similarly, “substantially perpendicular” is not limited to absolute perpendicularity, and can include orientations that are intended to be parallel but due to manufacturing limitations may not be precisely perpendicular. For example, “substantially perpendicular” features are at least closer to a perpendicular orientation than a parallel orientation, e.g., within a few degrees of perpendicular.

Where a compound disclosed herein has at least one chiral center, the compounds may accordingly exist as enantiomers. Where the compounds possess two chiral centers, the compounds may additionally exist as diastereomers. It is to be understood that all such isomers and mixtures thereof are encompassed within the scope of the present disclosure.

Unless otherwise required by context, singular terms shall include pluralities and plural terms shall include the singular. As used herein, the singular form “a”, “an”, and “the” include plural references unless indicated otherwise. For example, “an” excipient includes one or more excipients.

The details of one or more embodiments of the disclosure are set forth in the accompanying drawings and the description below. Other features, objects, and advantages of the disclosure will be apparent from the description and drawings, and from the claims. Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1A is a UV-VIS absorption spectrum of Compound 1 after the addition of L-lysine.

FIG. 1B shows the change in color of Compound 1 after the addition of L-lysine. Before the addition of L-lysine (e.g., when Time (h) is 0), the solution of Compound 1 appears colorless. After the addition of L-lysine (“Add Lysine”), the solution appears yellow. The solution gets progressively darker until it appears brown at 44 hours.

FIG. 2A shows the color of Compound 10 (oxetane) after the addition of L-lysine. The solution is a light brown color.

FIG. 2B is a UV-VIS absorption spectrum of Compound 10 after the addition of L-lysine, oxetane, or oxetane-lysine.

FIG. 3 is a UV-VIS absorption spectrum of lawsone after the addition of L-lysine and Compound 14 (Phen-Lawsone) after the addition of L-lysine.

FIG. 4A is a schematic side cross-sectional view of an exemplary article according to one embodiment of the present disclosure.

FIG. 4B is an exploded perspective view of an exemplary article according to another embodiment of the present disclosure.

FIG. 4C is a side cross-sectional view of the exemplary article of FIG. 4B.

FIG. 5 is an exploded perspective view of an adhesive applicator article according to another embodiment of the present disclosure.

FIG. 6 is a view of an adhesive applicator article according to another embodiment of the present disclosure.

FIG. 7 is an exploded perspective view of an adhesive applicator article according to another embodiment of the present disclosure.

FIG. 8 is another exploded perspective view of an adhesive applicator article according to another embodiment of the present disclosure.

FIGS. 9A, 9B, and 9C are perspective drawings of an adhesive applicator article according to an embodiment of the present disclosure.

FIGS. 10A, 10B, and 10C are perspective drawings of an adhesive applicator article according to an embodiment of the present disclosure.

FIGS. 11A, 11B, and 11C are perspective drawings of an adhesive applicator article according to an embodiment of the present disclosure.

FIGS. 12A, 12B, and 12C are perspective drawings of an adhesive applicator article according to an embodiment of the present disclosure.

FIGS. 13A, 13B, and 13C are perspective drawings of an adhesive applicator article according to an embodiment of the present disclosure.

FIG. 14 is another example of an adhesive applicator article according to another embodiment of the present disclosure.

FIGS. 15A and 15B illustrate perspective views of an example adhesive applicator article according to another embodiment of the present disclosure.

FIG. 16 is another example of an adhesive applicator article according to another embodiment of the present disclosure.

FIGS. 17A and 17B illustrate another example of an adhesive applicator article according to another embodiment of the present disclosure.

FIGS. 18A and 18B illustrate another example of an adhesive applicator article according to another embodiment of the present disclosure.

FIG. 19 is a UV-VIS absorption spectrum of Compound 11 (diethyl malonate-genipin) after the addition of L-lysine.

FIG. 20 is a UV-VIS absorption spectrum of Compound 12 (genipin carboxylic acid) before and after the addition of L-lysine.

FIG. 21 is a UV-VIS absorption spectrum of Compound 13 (genipin amide) before and after the addition of L-lysine.

FIG. 22 is a UV-VIS absorption spectrum of Compound 5 before and after the addition of L-lysine.

FIG. 23 is a UV-VIS absorption spectrum of Compound 7 before and after the addition of L-lysine.

FIG. 24 is a UV-VIS absorption spectrum of Compound 9 before and after the addition of L-lysine.

FIG. 25A is a UV-VIS absorption spectrum of genipin after the addition of L-lysine.

FIG. 25B shows the change in color of genipin after the addition of L-lysine. Before the addition of L-lysine, the solution of genipin appears colorless. After the addition of L-lysine (“Add Lysine”), the solution appears yellow. By 1 hr after the addition of the L-lysine, the solution appears dark blue. After dilution (vial at left), the solution appears light blue.

FIG. 26A is a UV-VIS absorption spectrum of Compound 1 (genipin aldehyde) after the addition of L-lysine.

FIG. 26B shows the change in color of Compound 1 after the addition of L-lysine. Before the addition of L-lysine, the solution of Compound 1 appears colorless. After the addition of L-lysine (“Lysine added”), the solution appears dark yellow. By 1 hour after the addition of the L-lysine, the solution appears dark brown. After dilution (vial at left), the solution appears yellow.

FIG. 27A is a UV-VIS absorption spectrum of Compound 12 after the addition of L-lysine.

FIG. 27B shows the change in color of Compound 12 after the addition of L-lysine. Before the addition of L-lysine, the solution of Compound 12 appears colorless. After the addition of L-lysine (“Lysine added”), the solution appears light yellow. The solution gets progressively darker until it appears dark brown at 18 hours. After dilution (vial at left), the solution appears light yellow.

FIG. 28A is a plot showing UV-VIS absorption spectra for genipin with L-lysine; Compound 1 with L-lysine; and a mixture of genipin and Compound 1 with L-lysine.

FIG. 28B shows the change in color of the mixture of genipin and Compound 1 after the addition of L-lysine. Before the addition of L-lysine, the solution of genipin and Compound 1 appears colorless. After the addition of L-lysine (“Lysine added”), the solution appears yellow. By 1 hr after the addition of the L-lysine, the solution appears black. After dilution (vial at left), the solution appears light green.

FIG. 29A is a plot showing UV-VIS absorption spectra for genipin with L-lysine; Compound 12 with L-lysine; and a mixture of genipin and Compound 12 with L-lysine.

FIG. 29B shows the change in color of mixture of genipin and Compound 12 after the addition of L-lysine. Before the addition of L-lysine, the solution of genipin and Compound 12 appears colorless. After the addition of L-lysine (“Lysine added”), the solution appears light yellow. By 1 hr after the addition of the L-lysine, the solution appears brown and by 2 hours after the addition of L-lysine, the solution appears black. After dilution (vial at left), the solution appears blue.

FIG. 30A is a plot showing UV-VIS absorption spectra for Compound 1 with L-lysine; Compound 12 with L-lysine; and a mixture of Compound 1 and Compound 12 with L-lysine.

FIG. 30B shows the change in color of the mixture of Compound 1 and Compound 12 after the addition of L-lysine. Before the addition of L-lysine, the solution of Compound 1 and Compound 12 appears colorless. After the addition of L-lysine (“Lysine added”), the solution appears light green. The solution gets progressively darker until it appears black at 18 hours. After dilution (vial at left), the solution appears light yellow.

FIG. 31A is a plot showing UV-VIS absorption spectra for genipin with L-lysine; Compound 1 with L-lysine; Compound 12 with L-lysine; and a mixture of genipin, Compound 1, and Compound 12 with L-lysine.

FIG. 31B shows the change in color of the mixture of genipin, Compound 1, and Compound 12 after the addition of L-lysine. Before the addition of L-lysine, the solution of genipin, Compound 1, and Compound 12 appears colorless. After the addition of L-lysine (“Lysine added”), the solution appears light tan. By 1 hr after the addition of the L-lysine, the solution appears black. After dilution (vial at left), the solution appears light green.

FIG. 32 is a plot showing UV-VIS absorption spectra for genipin with L-lysine; Compound 1 with L-lysine; Compound 12 with L-lysine; a mixture of genipin and Compound 1 with L-lysine (Mixture A); a mixture of genipin and Compound 12 with L-lysine (Mixture B); a mixture of Compound 1 and Compound 12 with L-lysine (Mixture C); and a mixture of genipin, Compound 1, and Compound 12 with L-lysine (Mixture D).

FIG. 33 shows the colors observed for genipin with L-lysine (light blue); Compound 1 with L-lysine (yellow); Compound 12 with L-lysine (light yellow); a mixture of genipin and Compound 1 with L-lysine (light green); a mixture of genipin and Compound 12 with L-lysine (blue); a mixture of Compound 1 and Compound 12 with L-lysine (light yellow); and a mixture of genipin, Compound 1, and Compound 12 with L-lysine (light green).

FIG. 34 has a plot showing UV-VIS absorption spectra for genipin+lawsone with L-lysine (left) and vials of solutions of genipin+lawsone with L-lysine (right).

FIG. 35 has a plot showing UV-VIS absorption spectra for Compound 1+lawsone with L-lysine (left) and vials of solutions of Compound 1+lawsone with L-lysine (right).

FIG. 36 has a plot showing UV-VIS absorption spectra for genipin+Compound 1+lawsone with L-lysine (left) and vials of solutions of genipin+Compound 1+lawsone with L-lysine (right).

FIG. 37 is a plot showing UV-VIS absorption spectra for genipin and Compound 1 with L-lysine; genipin and Compound 14 with L-lysine; Compound 1 and Compound 14 with L-lysine; genipin, Compound 1, and Compound 14 with L-lysine.

### DETAILED DESCRIPTION

Provided herein are semi-permanent colorants and semi-permanent colorant precursors that include derivatives of genipin and lawsone. Also provided herein are compositions including such semi-permanent colorants that can be applied to skin as a semi-permanent tattoo. Such compositions can be formulated to be safe for skin contact and sustain stability or skin transferability for the duration of the product shelf life. Also provided herein are layered adhesive articles and methods for application of the compositions described herein. In some embodiments, the compositions described herein can be suitable for application to skin using a pen-like applicator.

As used herein, a “semi-permanent colorant” refers to a colorant that penetrates one or more layers of the skin and is unable to be removed from the skin without physical disruption or natural desquamation of the skin. In some embodiments, a semi-permanent colorant can include a colorant precursor, for example, a molecule, such as genipin, that expresses color upon reaction with one or more other molecules. In some embodiments, a semi-permanent colorant may penetrate the stratum corneum and react with other molecules, e.g., other colorant molecules and/or other molecules present in the stratum corneum, such that the semi-permanent colorant is immobilized within the stratum corneum. For example, the semi-permanent colorant may cross-link with collagen or keratin found in the stratum corneum. In some embodiments, the semi-permanent colorant may react with other molecules, e.g., other colorant molecules and/or other molecules present in the stratum corneum, to form a molecule that is too large to diffuse out of the stratum corneum. In some embodiments, wherein the semi-permanent colorant penetrates the stratum corneum, the colorant residence time is determined by the natural skin desquamation process.

In some embodiments, the semi-permanent colorant cannot be washed off, for example, by water, soap, and/or isopropanol. In some embodiments, a semi-permanent colorant includes a natural or synthetic pigment or dye. Non-limiting examples of semi-permanent colorants include genipin, a genipin derivative, lawsone, and a lawsone derivative.

As used herein, “genipin” refers to genipin extract, partially purified genipin extract, and/or purified genipin, as described further herein. Genipin is derived from an iridoid glycoside called geniposide, which is present in nearly 40 plant species. Additionally, genipin is a precursor to gardenia blue that undergoes a transformative reaction upon exposure to amine groups to form a blue colorant (see, for example, Touyama et al., Chem. Pharm. Bull. 42:668-673, 1994; and Touyama et al., Chem. Pharm. Bull. 42(8):1571-1578, 1994; both of which are incorporated by reference herein in their entireties). When placed in contact with skin, genipin can react with the amines in skin to generate the polymer-based and/or oligomer-based blue color in situ.

A “genipin derivative” is a compound having a genipin core. For example, a genipin derivative is a compound having the following core structure:

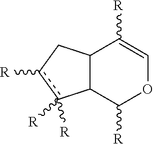

Non-limiting examples of genipin derivatives include compounds of Formula

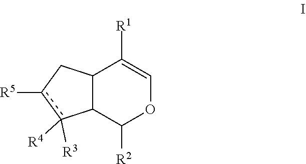

or a salt (e.g., pharmaceutically acceptable salt) thereof, wherein:
R¹ is —C(═O)OR⁶, C₁₋₆ alkyl, or C₆₋₁₀ aryl, wherein said C₁₋₆ alkyl and C₆₋₁₀ aryl are each optionally substituted with 1-5 R⁷;
R⁶ is H, C₁₋₆ alkyl, or C₆₋₁₀ aryl, wherein said C₁₋₆ alkyl and C₆₋₁₀ aryl are each optionally substituted with 1-5 R⁷;
each R⁷ is independently an electron withdrawing or an electron donating group;
R² is H, —OR⁹, or OC(═O)R^{9A};
R⁹ is selected from the group consisting of: H, C₁₋₆ alkyl, C₆₋₁₀ aryl, and a hydroxyl-protecting group;
R^{9A} is selected from C₁₋₆ alkyl and C₆₋₁₀ aryl; and
the dotted line indicates the optional presence of a double bond;
   wherein if the double bond is absent, then R⁴ and R⁵ together with the carbon atoms to which they are attached form a 3-4 membered heterocyclic ring and R³ is —OH or —CH₂OH; or
   if the double bond is present, then R⁴ and R⁵ are absent and R³ is selected from the group consisting of: —NR⁷R⁸, —CH₂NR⁷R⁸, —C(═O)OR¹⁰, —C(═O)N R¹⁰R¹⁰, —CH(═O), phenyl, —CHR¹⁰R¹⁰, —CH═CHR¹⁰, —CH═CR¹⁰R¹⁰, —CH₂OR¹⁰, and phenyl substituted with 1-5 R⁷;
   wherein R¹⁰ is selected from H, C₁₋₆ alkyl, alkoxy, oxo, —OH, —CH(═O), —C(═O)OR⁶, phenyl, and phenyl substituted with 1-5 R⁷; and
R⁸ is H or R⁷.

In some embodiments:
R¹ is —C(═O)OR⁶ or phenyl substituted with 1-5 R⁷;
R⁶ is H, C₁₋₆ alkyl, or phenyl substituted with 1-5 R⁷;
R² is H, —OR⁹, or OC(═O)R⁹;
R⁹ is H or C₁₋₆ alkyl;
if the double bond is present, then R⁴ and R⁵ are absent and R³ is selected from the group consisting of: —NR⁷R⁸, —CH₂NR⁷R⁸, —C(═O)OR¹⁰, —CH(═O), phenyl, —CHR¹⁰R¹⁰, —CH═CHR¹⁰, —CH₂OR¹⁰, diethyl malonate, and phenyl substituted with 1-5 R⁷; and
R¹⁰ is selected from H, C₁₋₆ alkyl, alkoxy, oxo, —OH, —CH(═O) and phenyl substituted with 1-5 R⁷.

In some embodiments:
R³ is selected from the group consisting of: —NR⁷R⁸, —CH₂NR⁷R⁸, —C(═O)OR¹⁰, —C(═O)N R¹⁰R¹⁰, —CH(═O), phenyl, —CHR¹⁰R¹⁰, —CH═CHR¹⁰, —CH═CR¹⁰R¹⁰, —CH₂OR¹⁰, and phenyl substituted with 1-5 R⁷;
R¹ is —C(═O)OH;
R² is —OH; and
R¹⁰ is selected from H, C₁₋₆ alkyl, alkoxy, oxo, —OH, —CH(═O), —C(═O)OR⁶, phenyl, and phenyl substituted with 1-5 R⁷.

Non-limiting examples of genipin derivatives include compounds of Formula

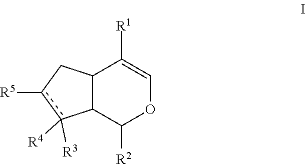

wherein:
R¹ is —C(═O)OR⁶ or phenyl substituted with 1-5 R⁷;
R⁶ is H, C₁₋₆ alkyl, or phenyl substituted with 1-5 R⁷;
each R⁷ is independently an electron withdrawing or an electron donating group;
R² is H, —OR⁹, or —OC(═O)R⁹;
R⁹ is H or C₁₋₆ alkyl; and
the dotted line indicates the optional presence of a double bond;
   wherein if the double bond is absent, then R⁴ and R⁵ together with the carbon atoms to which they are attached form a 3-4 membered heterocyclic ring and R³ is —OH or —CH₂OH; or
   if the double bond is present, then R⁴ and R⁵ are absent and R³ is selected from the group consisting of: —NR⁷R⁸, —CH₂NR⁷R⁸, —C(═O)OR¹⁶, —CH(═O), phenyl, —CHR¹⁰R¹⁰, —CH═CHR¹⁰, —CH₂OR¹⁰, diethyl malonate, and phenyl substituted with 1-5 R⁷;
   wherein each R¹⁰ is independently selected from H, C₁₋₆ alkyl, alkoxy, —OH, —CH(═O), and phenyl substituted with 1-5 R⁷; and
   R⁸ is H or R⁷.

In some embodiments, the Formula I has formula:

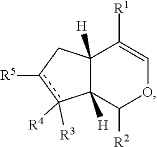

or a salt (e.g., pharmaceutically acceptable salt) thereof.

In some embodiments, the Formula I has formula

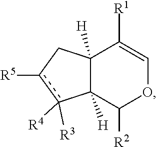

or a salt (e.g., pharmaceutically acceptable salt) thereof.

In some embodiments, the Formula I has formula

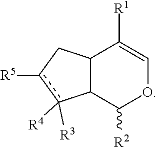

In some embodiments, the Formula I has formula:

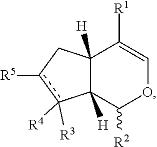

or a salt (e.g., pharmaceutically acceptable salt) thereof.

In some embodiments, R⁶ methyl. In some embodiments, R¹ is —C(═O)OR⁶ and R⁶ is methyl. In some embodiments, R¹ is —C(═O)OR⁶ and R⁶ is phenyl substituted with 1-5 R⁷.

In some embodiments, R² is —OC(═O)CH₃. In some embodiments, R² is —OH.

In some embodiments, R² is OR⁹ or OC(═O)R^{9A}.

In some embodiments, R² is OR⁹.

In some embodiments, R² is OC(═O)R^{9A}.

In some embodiments, R⁹ is selected from the group consisting of: H, C₁₋₆ alkyl, and C₆₋₁₀ aryl.

In some embodiments, R⁹ is a hydroxyl-protecting group. Suitable examples of protecting groups are described in Greene, Wuts, at al., Protective groups in organic synthesis, Fourth Edition, 2006 (ISBN: 9780471697541), and include t-butyl, allyl, methoxymethyl (MOM), tri(C₁₋₆ alkyl)silyl (e.g., tert-butyldimethylsilyl (TBS), trimethylsilyl (TMS), or triethylsilyl (TES)), acyl (e.g., benzoyl, pivaloyl, or acetyl), acetonide, tetrahydropyranyl (THP), and benzylidene acetal. In some embodiments, the hydroxyl-protecting group is tri(C₁₋₆ alkyl)silyl. In some embodiments, the hydroxyl-protecting group is tert-butyldimethylsilyl (TBS), trimethylsilyl (TMS), or triethylsilyl (TES).

In some embodiments, R^{9A} is C₁₋₆ alkyl.

In some embodiments, R^{9A} is C₆₋₁₀ aryl.

In some embodiments, the double bond is absent. In some embodiments, R⁴ and R⁵ together with the carbon atoms to which they are attached form a 3-membered heterocyclic ring and R³ is —CH₂OH. For example, R⁴ and R⁵ together with the carbon atoms can form:

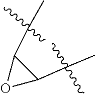 [SMILES: *C1OC1*]

In some embodiments, R⁴ and R⁵ together with the carbon atoms to which they are attached form a 4-membered heterocyclic ring and R³ is —OH. For example, R⁴ and R⁵ together with the carbon atoms can form:

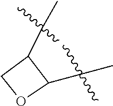 [SMILES: *C1COC1*]

In some embodiments, the double bond is present. In some embodiments, R¹ is phenyl substituted with 1-3 R⁷. In some embodiments, R¹ is —C(═O)OR⁶ and R⁶ is phenyl substituted with 1-3 R⁷. In some embodiments, R² is —OH and R³ is CH₂OH.

In some embodiments, R¹ is C₆₋₁₀ aryl, optionally substituted with 1-5 R⁷.

In some embodiments, R⁶ is C₆₋₁₀ aryl, optionally substituted with 1-5 R⁷.

In some embodiments, R³ is selected from the group consisting of: —NR⁷R⁸, —CH₂—NR⁷R⁸, —C(═O)OR¹⁰, —CH(═O), phenyl, —CHR¹⁰R¹⁰, —CH═CHR¹⁰, —CH₂OR¹⁰, diethyl malonate, and phenyl substituted with 1-5 R⁷; R¹ is —C(═O)OH; and R² is —OH.

In some embodiments, R³ is selected from —CH₂NR⁷R⁸, —C(═O)OR¹⁰, —C(═O)N R¹⁰R¹⁰, —CH(═O), phenyl, —CH(OH)R¹⁰, —CH═CHR¹⁰, —CH═CR¹⁰R¹⁰, —CH₂OR¹⁰, and phenyl substituted with 1-5 R⁷.

In some embodiments, R¹⁰ is selected from H, C₁₋₆ alkyl, —OH, —CH(═O), —C(═O)OR⁶, phenyl, and phenyl substituted with 1-5 R⁷.

In some embodiments, R¹⁰ is selected from H, C₁₋₆ alkyl, —CH(═O), —C(═O)OR⁶, phenyl, and phenyl substituted with 1-5 R⁷.

In some embodiments, R¹⁰ is selected from H, C₁₋₆ alkyl, phenyl, and phenyl substituted with 1-5 R⁷.

In some embodiments, R¹⁰ is H.

In some embodiments, R¹⁰ is selected from C₁₋₆ alkyl, phenyl, and phenyl substituted with 1-5 R⁷.

In some embodiments, R³ is —CH(OH)R¹⁰; and R¹⁰ is C₁₋₆ alkyl, phenyl, or phenyl substituted with 1-5 R⁷.

In some embodiments, R³ is —C(═O)N R¹⁰R¹⁰.

In some embodiments, R³ is —CH═CHR¹⁰; and R¹⁰ is C₁₋₆ alkyl, phenyl, or phenyl substituted with 1-5 R⁷. In some embodiments, R¹⁰ is phenyl or phenyl substituted with 1-5 R⁷.

In some embodiments, R³ is phenyl.

In some embodiments, R³ is phenyl substituted with 1-5 R⁷.

In some embodiments, R³ is —CH═CR¹⁰R¹⁰; each R¹⁰ is independently —CH(═O) or —C(═O)OR⁶; and R⁶ is C₁₋₆ alkyl, optionally substituted with 1-5 R⁷. In some embodiments, R³ is diethyl malonate.

In some embodiments, the genipin derivative is a compound selected from the group consisting of the compounds in Table 1.

TABLE 1

| Compound # | Structure |
|---|---|
| 1 | 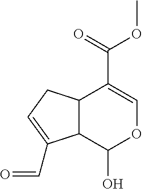 [SMILES: COC(=O)C1=COC(O)C2C(C=O)=CCC12] |
| 2 | 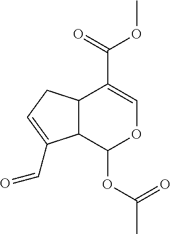 [SMILES: COC(=O)C1=COC(OC(C)=O)C2C(C=O)=CCC12] |
| 3 | 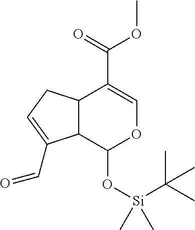 [SMILES: COC(=O)C1=COC(O[Si](C)(C)C(C)(C)C)C2C(C=O)=CCC12] |
| 4 | 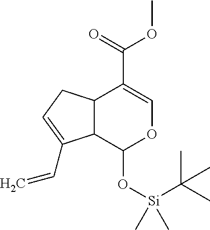 [SMILES: C=CC1=CCC2C(C(=O)OC)=COC(O[Si](C)(C)C(C)(C)C)C12] |
| 5 | 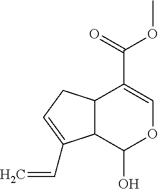 [SMILES: C=CC1=CCC2C(C(=O)OC)=COC(O)C12] |
| 6 | 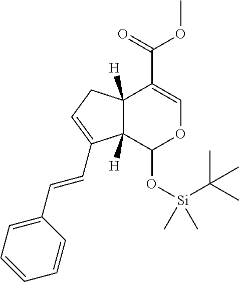 [SMILES: COC(=O)C1=COC(O[Si](C)(C)C(C)(C)C)[C@@H]2C(/C=C/c3ccccc3)=CC[C@H]12] |
| 7 | 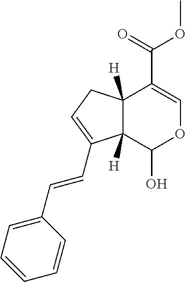 [SMILES: COC(=O)C1=COC(O)[C@@H]2C(/C=C/c3ccccc3)=CC[C@H]12] |
| 8 | 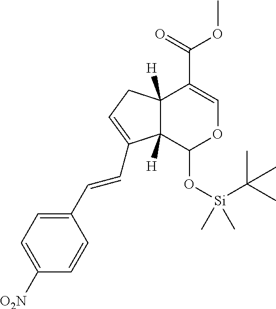 [SMILES: COC(=O)C1=COC(O[Si](C)(C)C(C)(C)C)[C@@H]2C(/C=C/c3ccc([N+](=O)[O-])cc3)=CC[C@H]12] |
| 9 | 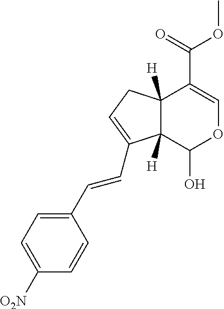 [SMILES: COC(=O)C1=COC(O)[C@@H]2C(/C=C/c3ccc([N+](=O)[O-])cc3)=CC[C@H]12] |
| 10 | 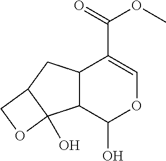 [SMILES: COC(=O)C1=COC(O)C2C1CC1COC12O] |
| 11 | 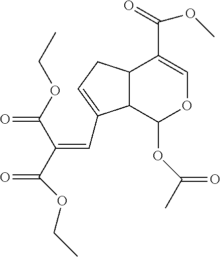 [SMILES: CCOC(=O)C(=CC1=CCC2C(C(=O)OC)=COC(OC(C)=O)C12)C(=O)OCC] |
| 12 | 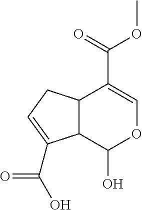 [SMILES: COC(=O)C1=COC(O)C2C(C(=O)O)=CCC12] |
| 13 | 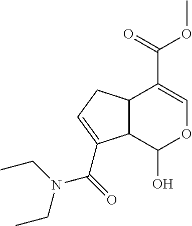 [SMILES: CCN(CC)C(=O)C1=CCC2C(C(=O)OC)=COC(O)C12] |
| 15 | 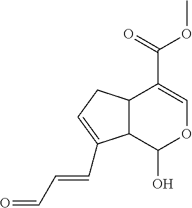 [SMILES: COC(=O)C1=COC(O)C2C(/C=C/C=O)=CCC12] |
| 16 | 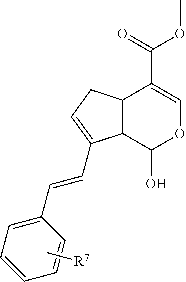 |
| 17 | 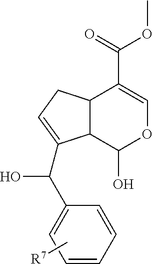 |
| 18 | 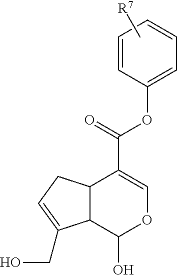 |
| 19 | 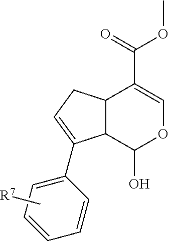 |
| 20 | 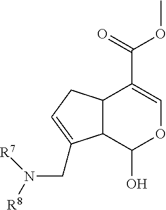 |
| 21 | 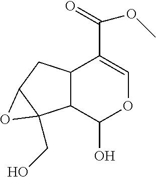 [SMILES: COC(=O)C1=COC(O)C2C1CC1OC12CO] |
| 22 | 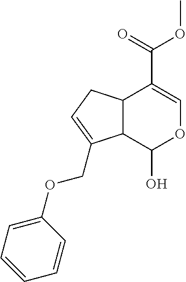 [SMILES: COC(=O)C1=COC(O)C2C(COc3ccccc3)=CCC12] |
| 23 | 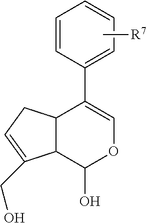 |

In some embodiments, the electron withdrawing or electron donating group of R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, halide, and —NO. In some embodiments, the electron withdrawing or electron donating group of R⁷ is selected from the group consisting of: —(C₁₋₉ alkyl), —(C₂₋₉ alkenyl), phenyl, —NR⁸R⁸, —OR⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, and —NO.

In some embodiments, R⁸ is H or R⁷. In some embodiments, R⁸ is selected from the group consisting of: H, —(C₁₋₉ alkyl), and —(C₂₋₉ alkenyl); and R⁹ is halide. In some embodiments, R⁸ is H.

In some embodiments, the present disclosure provides a genipin derivative which has Formula IIa:

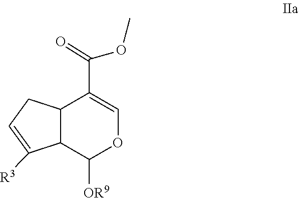

or a salt (e.g., a pharmaceutically acceptable salt) thereof, wherein:
R⁹ is selected from the group consisting of: H, C₁₋₆ alkyl, C₆₋₁₀ aryl, and a hydroxyl-protecting group, wherein said C₁₋₆ alkyl and C₆₋₁₀ aryl are each optionally substituted with 1-5 R⁷;
R³ is selected from the group consisting of: —CH₂NR⁷R⁸, —C(═O)OR¹⁰, —C(═O)N R¹⁰R¹⁰, —CH(═O), —CHR¹⁰R¹⁰, —CH═CHR¹⁰, —CH═CR¹⁰R¹⁰, —CH₂OR¹⁰, phenyl, and phenyl substituted with 1-5 R⁷;
wherein each R¹⁰ is independently selected from H, C₁₋₆ alkyl, —OH, —CH(═O), C(═O)OR⁹, phenyl, and phenyl substituted with 1-5 R⁷; and
R⁷ is an electron withdrawing or an electron donating group; and
R⁸ is H or R⁷.

In some embodiments:
R³ is selected from —C(═O)OR¹⁰, —CH(═O), and —CH₂OR¹⁰;
R¹⁰ is selected from H, C₁₋₆ alkyl, phenyl, and phenyl substituted with 1-5 R⁷;
and
R⁹ is selected from C₁₋₆ alkyl, C₆₋₁₀ aryl, and a hydroxyl-protecting group, wherein said C₁₋₆ alkyl and C₆₋₁₀ aryl are each optionally substituted with 1-5 R⁷.

In some embodiments, R³ is —C(═O)OR¹⁰.

In some embodiments, R³ is —CH(═O).

In some embodiments, R³ is —CH₂OR¹⁰.

In some embodiments:
R³ is —CH═CR¹⁰R¹⁰;
each R¹⁰ is independently CH(═O) or C(═O)OR⁹; and
R⁹ is selected from C₁₋₆ alkyl, C₆₋₁₀ aryl, and a hydroxyl-protecting group, wherein said C₁₋₆ alkyl and C₆₋₁₀ aryl are each optionally substituted with 1-5 R⁷.

In some embodiments:
R³ is selected from —CH₂NR⁷R⁸, —C(═O)N R¹⁰R¹⁰, —CH(OH)R¹⁰, —CH═CHR¹⁰, phenyl, and phenyl substituted with 1-5 R⁷; and
each R¹⁰ is independently selected from C₁₋₆ alkyl, phenyl, and phenyl substituted with 1-5 R⁷.

In some embodiments, R⁹ is a hydroxyl-protecting group. In some embodiments, R9 is selected from the group consisting of: t-butyl, allyl, methoxymethyl, tri(C₁₋₆ alkyl)silyl (e.g., tert-butyldimethylsilyl (TBS), trimethylsilyl (TMS), or triethylsilyl (TES)), acyl (e.g., benzoyl, pivaloyl, or acetyl), acetonide, and benzylidene acetal. In some embodiments, the hydroxyl-protecting group is tri(C₁₋₆ alkyl)silyl. In some embodiments, the hydroxyl-protecting group is tert-butyldimethylsilyl (TBS), trimethylsilyl (TMS), or triethylsilyl (TES).

In some embodiments, R⁹ is C₁₋₆ alkyl.

In some embodiments, R⁹ is C₆₋₁₀ aryl.

In some embodiments, the Formula IIa has formula:

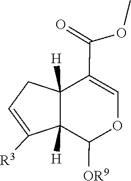

or a salt (e.g., a pharmaceutically acceptable salt) thereof.

In some embodiments, the Formula IIa has formula:

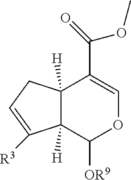

or a salt (e.g., a pharmaceutically acceptable salt) thereof.

In some embodiments, the Formula IIa has formula:

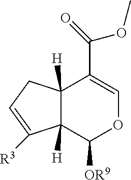

or a salt (e.g., a pharmaceutically acceptable salt) thereof.

In some embodiments, the Formula IIa has formula:

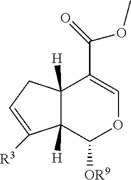

or a salt (e.g., a pharmaceutically acceptable salt) thereof.

In some embodiments, the Formula IIa has formula:

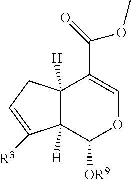

or a salt (e.g., a pharmaceutically acceptable salt) thereof.

In some embodiments, the Formula IIa has formula:

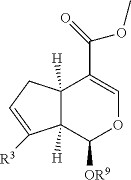

or a salt (e.g., a pharmaceutically acceptable salt) thereof.

In some embodiments, the genipin derivative is a compound of Formula IIa:

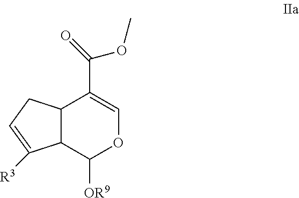

wherein:
R⁹ is selected from the group consisting of: H, C₁₋₆ alkyl, C₆₋₁₀ aryl, and a hydroxyl-protecting group, wherein said C₁₋₆ alkyl and C₆₋₁₀ aryl are each optionally substituted with 1-5 R⁷;
R³ is selected from the group consisting of: —C(═O)OR¹⁰, —CH(═O), —CHR¹⁰R¹⁰, —CH═CHR¹⁰, —CH₂OR¹⁰, —C(═O)NR¹⁰R¹⁰, phenyl, and phenyl substituted with 1-5 R⁷;
   wherein each R¹⁰ is independently selected from H, —OH, —CH(═O), diethyl malonate, phenyl, and phenyl substituted with 1-5 R⁷; and
   R⁷ is an electron withdrawing or an electron donating group, e.g., any of the electron withdrawing or an electron donating group described herein.

In some embodiments, R⁹ is tert-butyldimethylsilyl (TBS). In some embodiments, R³ is selected from the group consisting of: —C(═O)OR¹⁰, —CH(═O), —CH═CHR¹⁰, —CH₂OR¹⁰, and —C(═O)NR¹⁰R¹⁰. In some embodiments, each R¹⁰ is independently selected from H, C₁₋₆ alkyl, —CH(═O), -phenyl, and phenyl substituted with 1-5 R⁷. In some embodiments, R¹⁰ is phenyl substituted with —NO₂. In some embodiments, R³ is —C(═O)OR¹⁰ and R¹⁰ is H. In some embodiments, R³ is —CH═CHR¹⁰ and R¹⁰ is selected from the group consisting of: H, CH(═O), phenyl, and phenyl substituted with 1-5 R⁷. In some embodiments, R³ is —CH═CHR¹⁰ and R¹⁰ is phenyl substituted with —NO₂. In some embodiments, R³ is —CH₂OR¹⁰ and R¹⁰ is H or phenyl. In some embodiments, R³ is C(═O)NR¹⁰R¹⁰ and R¹⁰ is C₁₋₆ alkyl. In some embodiments, R³ is C(═O)NR¹⁰R¹⁰ and R¹⁰ is ethyl.

In some embodiments, R⁹ is —OH. In some embodiments, R³ is selected from the group consisting of: —C(═O)OR¹⁰, —CH(═O), —CH═CHR¹⁰, —CH₂OR¹⁰, and —C(═O)NR¹⁰R¹⁰. In some embodiments, each R¹⁰ is independently selected from H, C₁₋₆ alkyl, —CH(═O), -phenyl, and phenyl substituted with 1-5 R⁷. In some embodiments, R¹⁰ is phenyl substituted with —NO₂. In some embodiments, R³ is —C(═O)OR¹⁰ and R¹⁰ is H. In some embodiments, R³ is —CH═CHR¹⁰ and R¹⁰ is selected from the group consisting of: H, CH(═O), phenyl, and phenyl substituted with 1-5 R⁷. In some embodiments, R³ is —CH═CHR¹⁰ and R¹⁰ is phenyl substituted with —NO₂. In some embodiments, R³ is —CH₂OR¹⁰ and R¹⁰ is H or phenyl. In some embodiments, R³ is C(═O)NR¹⁰R¹⁰ and R¹⁰ is C₁₋₆ alkyl. In some embodiments, R³ is C(═O)NR¹⁰R¹⁰ and R¹⁰ is ethyl.

In some embodiments, the genipin derivative is a compound as delineated in Table 2.

TABLE 2

| Compound # | Structure |
|---|---|
| 1 | 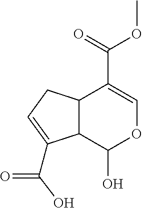 [SMILES: COC(=O)C1=COC(O)C2C(C(=O)O)=CCC12] |
| 3 | 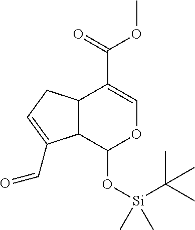 [SMILES: COC(=O)C1=COC(O[Si](C)(C)C(C)(C)C)C2C(C=O)=CCC12] |
| 4 | 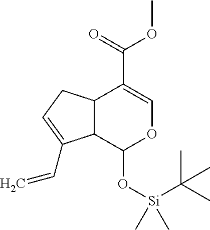 [SMILES: C=CC1=CCC2C(C(=O)OC)=COC(O[Si](C)(C)C(C)(C)C)C12] |
| 5 | 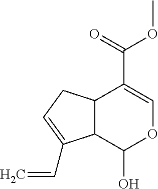 [SMILES: C=CC1=CCC2C(C(=O)OC)=COC(O)C12] |
| 6 | 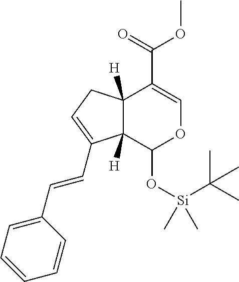 [SMILES: COC(=O)C1=COC(O[Si](C)(C)C(C)(C)C)[C@@H]2C(/C=C/c3ccccc3)=CC[C@H]12] |
| 7 | 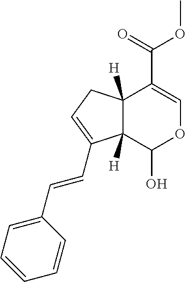 [SMILES: COC(=O)C1=COC(O)[C@@H]2C(/C=C/c3ccccc3)=CC[C@H]12] |
| 8 | 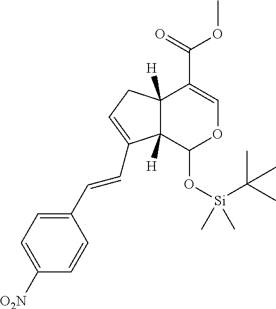 [SMILES: COC(=O)C1=COC(O[Si](C)(C)C(C)(C)C)[C@@H]2C(/C=C/c3ccc([N+](=O)[O-])cc3)=CC[C@H]12] |
| 9 | 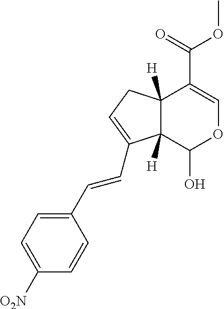 [SMILES: COC(=O)C1=COC(O)[C@@H]2C(/C=C/c3ccc([N+](=O)[O-])cc3)=CC[C@H]12] |
| 12 | 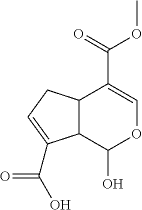 [SMILES: COC(=O)C1=COC(O)C2C(C(=O)O)=CCC12] |
| 13 | 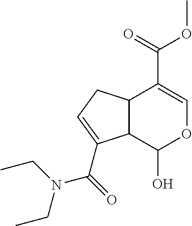 [SMILES: CCN(CC)C(=O)C1=CCC2C(C(=O)OC)=COC(O)C12] |
| 15 | 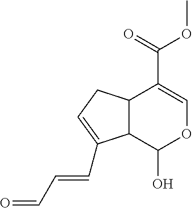 [SMILES: COC(=O)C1=COC(O)C2C(/C=C/C=O)=CCC12] |
| 16 | 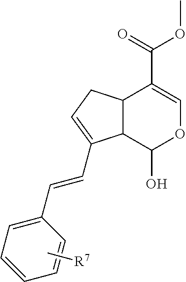 |
| 17 | 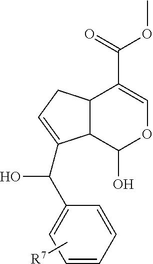 |
| 19 | 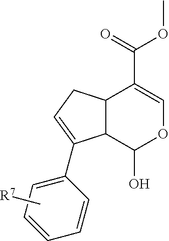 |
| 20 | 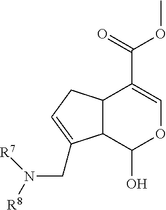 |
| 22 | 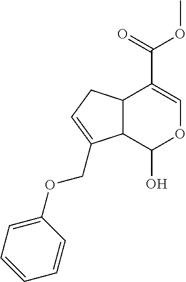 [SMILES: COC(=O)C1=COC(O)C2C(COc3ccccc3)=CCC12] |

In some embodiments, the compound has formula IIb:

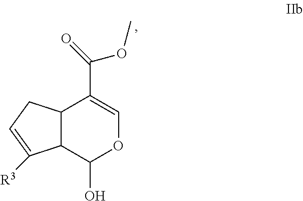

or a pharmaceutically acceptable salt thereof.

In some embodiments:
R³ is selected from the group consisting of:—CH₂NR⁷R⁸, —C(═O)OR¹⁰, —CH(═O), —CHR¹⁰R¹⁰, —CH═CHR¹⁰, —CH₂OR¹⁰, and phenyl substituted with 1-5 R⁷;
each R¹⁰ is independently selected from H, —OH, —CH(═O) and phenyl substituted with 1-5 R⁷.

In some embodiments, the genipin derivative is a compound of Formula IIb:

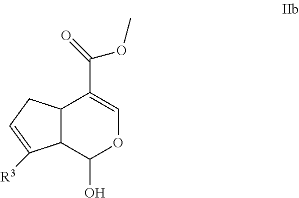

wherein:
R³ is selected from the group consisting of: —CH₂NR⁷R⁸, —C(═O)OR¹⁰, —CH(═O), —CHR¹⁰R¹⁰, —CH═CHR¹⁰, —CH₂OR¹⁰, and phenyl substituted with 1-5 R⁷;
   wherein each R¹⁰ is independently selected from H, —OH, —CH(═O), and phenyl substituted with 1-5 R⁷; and
   R⁷ is an electron withdrawing or an electron donating group, e.g., any of the electron withdrawing or an electron donating group described herein.

In some embodiments, R³ is selected from the group consisting of: —C(═O)OR¹⁰, —CH(═O), —CH═CHR¹⁰, —CH₂OR¹⁰, and —C(═O)NR¹⁰R¹⁰.

In some embodiments, each R¹⁰ is independently selected from H, C₁₋₆ alkyl, —CH(═O), -phenyl, and phenyl substituted with 1-5 R⁷. In some embodiments, R¹⁰ is phenyl substituted with —NO₂.

In some embodiments, R³ is —C(═O)OR¹⁰ and R¹⁰ is H.

In some embodiments, R³ is —CH═CHR¹⁰ and R¹⁰ is selected from the group consisting of: H, CH(═O), phenyl, and phenyl substituted with 1-5 R⁷. In some embodiments, R³ is —CH═CHR¹⁰ and R¹⁰ is phenyl substituted with —NO₂.

In some embodiments, R³ is —CH₂OR¹⁰ and R¹⁰ is H or phenyl.

In some embodiments, R³ is C(═O)NR¹⁰R¹⁰ and R¹⁰ is C₁₋₆ alkyl.

In some embodiments, R³ is C(═O)NR¹⁰R¹⁰ an d R¹⁰ is ethyl.

In some embodiments, the genipin derivative is a compound as delineated in Table 3.

TABLE 3

| Compound # | Structure |
|---|---|
| 1 | 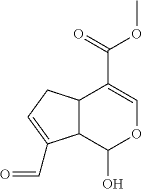 [SMILES: COC(=O)C1=COC(O)C2C(C=O)=CCC12] |
| 5 | 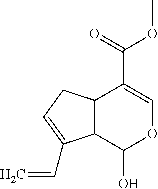 [SMILES: C=CC1=CCC2C(C(=O)OC)=COC(O)C12] |
| 7 | 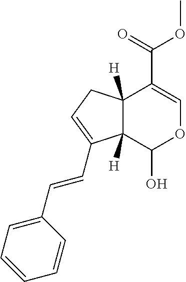 [SMILES: COC(=O)C1=COC(O)[C@@H]2C(/C=C/c3ccccc3)=CC[C@H]12] |
| 9 | 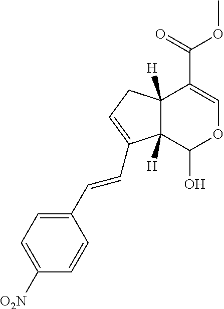 [SMILES: COC(=O)C1=COC(O)[C@@H]2C(/C=C/c3ccc([N+](=O)[O-])cc3)=CC[C@H]12] |
| 12 | 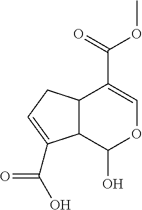 [SMILES: COC(=O)C1=COC(O)C2C(C(=O)O)=CCC12] |
| 13 | 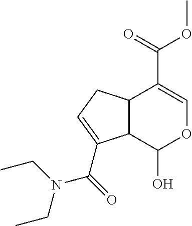 [SMILES: CCN(CC)C(=O)C1=CCC2C(C(=O)OC)=COC(O)C12] |
| 15 | 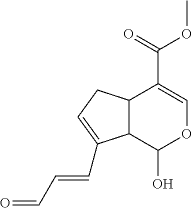 [SMILES: COC(=O)C1=COC(O)C2C(/C=C/C=O)=CCC12] |
| 16 | 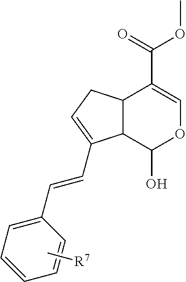 |
| 17 | 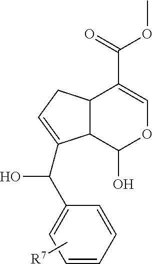 |
| 19 | 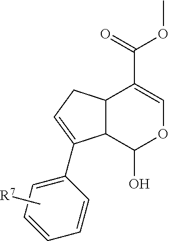 |
| 20 | 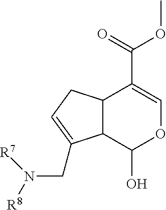 |
| 22 | 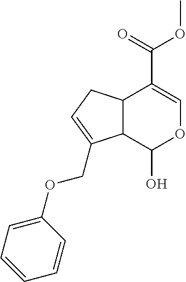 [SMILES: COC(=O)C1=COC(O)C2C(COc3ccccc3)=CCC12] |

In some embodiments, R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, halide, and —NO; wherein R⁸ is selected from the group consisting of: H, —(C₁₋₉ alkyl), —(C₂₋₉ alkenyl), unsubstituted —(C₂₋₉ alkynyl); and R⁹ is halide. In some embodiments, R⁷ is selected from the group consisting of: —(C₁₋₉ alkyl), —(C₂₋₉ alkenyl), phenyl, —NR⁸R⁸, —OR⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, and —NO.

In some embodiments, the chiral carbon atoms in the Formula IIb have the same configuration as in Formula IIa (R or S).

Without being bound by any theory, it is believed that the penetration profile of genipin or a genipin derivative can dictate the color concentration profile in the stratum corneum as the polymer-based and/or oligomer-based colorant is too large to penetrate the skin at relevant concentrations. For example, the average molecular weight of the polymer-based and/or oligomer-based blue colorant from genipin has been reported to be 8900±600 Daltons, and the polymer-based and/or oligomer-based blue colorant comprises 40 to 44 monomer units (see, for example, Touyama et al., Chem. Pharm. Bull. 42:668-673, 1994). In some embodiments, the average molecular weight of the monomeric, polymerized and/or oligomerized genipin or a genipin derivative present in the compositions described herein can range from about 200 Daltons to about 15000 Daltons. In some embodiments, the average molecular weight of the polymerized and/or oligomerized genipin or a genipin derivative present in the compositions described herein can range from about 500 Daltons to about 15000 Daltons. In some embodiments, a plurality of different molecular weights of genipin or a genipin derivative can be used. For example, in some embodiments, the compositions described herein can comprise genipin having an average molecular weight from about 200 Daltons to about 15000 Daltons. In some embodiments, the compositions described herein can comprise genipin or a genipin derivative having an average molecular weight of from about 200 to about 1000 Daltons, about 500 to about 1500 Daltons, about 1000 to about 2000 Daltons, about 1500 to about 2500 Daltons, about 2000 to about 3500 Daltons, 3500 to about 5000 Daltons, 5000 to about 6500 Daltons, 6000 to about 8300 Daltons, from about 8300 to about 9500 Daltons, and combinations thereof.

As used herein, “lawsone” refers to 2-hydroxy-1,4-naphthoquinone, also known as hennotannic acid. Lawsone is a red-orange dye present in the leaves of the henna plant (Lawsonia inermis) and in the flower of the water hyacinth (Eichhornia crassipes). Lawsone reacts with proteins such as keratin in skin and hair, and the semi-permanent colorant lasts until the skin or hair is shed.

As used herein, “lawsone derivative” refers to a compound having a lawsone core. For example, a lawsone derivative is a compound having the following core structure:

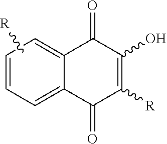

A non-limiting example of a lawsone derivative is the compound as delineated in Table 4.

TABLE 4

| Compound # | Structure |
|---|---|
| 14 | 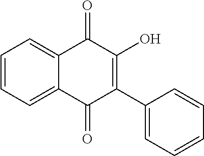 [SMILES: O=C1C(O)=C(c2ccccc2)C(=O)c2ccccc21] |

Further non-limiting examples of lawsone derivatives include compounds of Formula III:

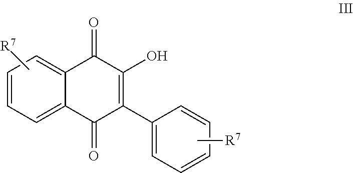

wherein R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, halogen, and —NO; wherein R⁸ is selected from the group consisting of: H, —(C₁₋₉ alkyl), and —(C₂₋₉ alkenyl); and R⁹ is halide.

In some embodiments, the electron withdrawing or electron donating group of R⁷ is selected from the group consisting of: —(C₁₋₉ alkyl), —(C₂₋₉ alkenyl), phenyl, —NR⁸R⁸, —OR⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, and —NO.

In some embodiments, the semi-permanent colorant (e.g., genipin derivative, lawsone, lawsone derivative, or combinations thereof) is purified. The presence of amines, for example, amines that are endogenously present in plant extracts or exogenously introduced, in a genipin or other semi-permanent colorant solution can shorten the shelf life of the semi-permanent colorant solution. For example, with time, the reactive precursors can develop into the polymer-based and/or oligomer-based colorant prior to application to the skin, limiting the penetration of the semi-permanent colorant to the stratum corneum. Penetration of the genipin or other semi-permanent colorant into the deeper layers of the stratum corneum can allow for the subsequent development and retention of the semi-permanent colorant (e.g., polymer-based and/or oligomer-based semi-permanent colorant) in the skin, which can lead to increased semi-permanent tattoo longevity. The semi-permanent tattoo longevity, or the semi-permanent colorant residence time in the skin, can then be limited by the natural skin desquamation process. In some embodiments, the purified semi-permanent colorant has a purity of greater than about 85% w/w. For example, the purified semi-permanent colorant has a purity of greater than about 86%, about 88%, about 90%, about 92%, about 94%, about 96%, about 98%, about 99%, or about 99.5% w/w of the semi-permanent colorant solution.

In some embodiments, a salt of any of the compounds described herein is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of this disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-I,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, p-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of this disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Provided herein are compositions comprising a semi-permanent colorant (e.g., a genipin derivative, lawsone, a lawsone derivative, or a combination thereof, as described herein). In some embodiments, the semi-permanent colorant comprises a compound of Formula I, a compound of Formula II, a compound of Formula III, or a combination thereof, wherein the Formulae I, II, and III are as described herein. In some embodiments, the semi-permanent colorant comprises a compound of Formula IIb:

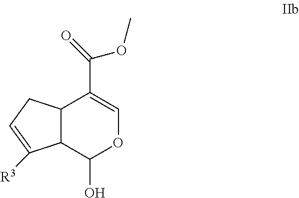

wherein:
R³ is selected from the group consisting of:—CH₂NR⁷R⁸, —C(═O)OR¹⁰, —CH(═O), —CHR¹⁰R¹⁰, —CH═CHR¹⁰, —CH₂OR¹⁰, and phenyl substituted with 1-5 R⁷;
   wherein each R¹⁰ is independently selected from H, —OH, —CH(═O) and phenyl substituted with 1-5 R⁷; and
   R⁷ is an electron withdrawing or an electron donating group, e.g., any of the electron withdrawing or an electron donating group described herein.

In some embodiments, the semi-permanent colorant comprises one or more compounds selected from the group consisting of the compounds as delineated in Table 1, Table 2, or Table 3. In some embodiments, the semi-permanent colorant is a compound selected from the group consisting of the compounds as delineated in Table 1, Table 2, or Table 3. In some embodiments, R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, halide, and —NO; wherein R⁸ is selected from the group consisting of: H, —(C₁₋₉ alkyl), —(C₂₋₉ alkenyl), unsubstituted —(C₂₋₉ alkynyl); and R⁹ is halide. In some embodiments, the electron withdrawing or electron donating group of R⁷ is selected from the group consisting of: —(C₁₋₉ alkyl), —(C₂₋₉ alkenyl), phenyl, —NR⁸R⁸, —OR⁸, —OC(═O)R⁸, —SR⁸, —CO₂⁻, —SO₂CF₃, —NO₂, —SO₂R⁸, —CN, —CR⁹₃, —COR⁹, —CHO, —CO₂R⁸, —C(═O)NR⁸R⁸, and —NO.

In some embodiments, the semi-permanent colorant further comprises genipin.

In some embodiments, the semi-permanent colorant is a combination of two or more semi-permanent colorants (e.g., a combination of two or more of genipin, a genipin derivative, lawsone, and a lawsone derivative). A combination of two or more semi-permanent colorants can include, for example, a combination of two or more genipin derivatives; a combination of one or more genipin derivatives and lawsone; a combination of one or more genipin derivatives and one or more lawsone derivatives; a combination of lawsone and one or more lawsone derivatives; and a combination of one or more genipin derivatives, lawsone, and one or more lawsone derivatives. In some embodiments, the semi-permanent colorant comprises a combination of two or more of: a compound of Formula I, a compound of Formula IIa, a compound of Formula IIb, and a compound of Formula III.

In some embodiments, the semi-permanent colorant comprises two semi-permanent colorants, e.g., a first semi-permanent colorant and a second semi-permanent colorant. For example, the semi-permanent colorant can comprise genipin and a genipin derivative or the semi-permanent colorant can comprise two different genipin derivatives (e.g., any two of the genipin derivatives described herein). In some embodiments, the first semi-permanent colorant is genipin and the second semi-permanent colorant is Compound 1. In some embodiments, the first semi-permanent colorant is genipin and the second semi-permanent colorant is Compound 12. In some embodiments, the first semi-permanent colorant is Compound 1 and the second semi-permanent colorant is Compound 12. In some embodiments, the first semi-permanent colorant is genipin and the second semi-permanent colorant is Compound 14. In some embodiments, the first semi-permanent colorant is Compound 1 and the second semi-permanent colorant is Compound 14. In some embodiments, the first semi-permanent colorant is Compound 7 and the second semi-permanent colorant is Compound 9. In some embodiments, the first semi-permanent colorant is Compound 7 and the second semi-permanent colorant is Compound 16. In some embodiments, the first semi-permanent colorant is Compound 9 and the second semi-permanent colorant is Compound 16.

In some embodiments, the first semi-permanent colorant and the second semi-permanent colorant are present at a ratio of about 1:4 to about 4:1 first semi-permanent colorant:second semi-permanent colorant. For example, about 1:4 to about 1:3, about 1:4 to about 1:2, about 1:4 to about 1:1, about 1:4 to about 2:1, about 1:4 to about 3:1, about 3:1 to about 4:1, about 2:1 to about 4:1, about 1:1 to about 4:1, about 1:2 to about 4:1, or about 1:3 to about 4:1 first semi-permanent colorant:second semi-permanent colorant. In some embodiments, the first semi-permanent colorant and the second semi-permanent colorant are present at a ratio of about 1:4, about 1:3, about 1:2, about 1:1, about 2:1, about 1:1, about 2:1, about 3:1, or about 4:1 first semi-permanent colorant:second semi-permanent colorant. In some embodiments, the first semi-permanent colorant and the second semi-permanent colorant are present at a ratio of about 1:1 first semi-permanent colorant:second semi-permanent colorant.

In some embodiments, the semi-permanent colorant comprises three semi-permanent colorants, e.g., a first semi-permanent colorant, a second semi-permanent colorant, and a third semi-permanent colorant. For example, the semi-permanent colorant can comprise genipin and two different genipin derivatives or the semi-permanent colorant can comprise three different genipin derivatives (e.g., any three of the genipin derivatives described herein). In some embodiments, the first semi-permanent colorant is genipin, the second semi-permanent colorant is Compound 1, and the third semi-permanent colorant is Compound 12. In some embodiments, the first semi-permanent colorant is genipin, the second semi-permanent colorant is Compound 1, and the third semi-permanent colorant is Compound 14. In some embodiments, the first semi-permanent colorant is Compound 7, the second semi-permanent colorant is Compound 9, and the third semi-permanent colorant is Compound 16.

In some embodiments, the first semi-permanent colorant, the second semi-permanent colorant, and the third semi-permanent colorant are present at a ratio of about 1:4:1 to about 4:1:1 first semi-permanent colorant:second semi-permanent colorant:third semi-permanent colorant. For example, about 1:4:1 to about 1:3:1, about 1:4:1 to about 1:2:1, about 1:4:1 to about 1:1:1, about 1:4:1 to about 2:1:1, about 1:4:1 to about 3:1:1, about 3:1:1 to about 4:1:1, about 2:1:1 to about 4:1:1, about 1:1:1 to about 4:1:1, about 1:2:1 to about 4:1:1, or about 1:3:1 to about 4:1:1 first semi-permanent colorant:second semi-permanent colorant:third semi-permanent colorant. In some embodiments, the first semi-permanent colorant, the second semi-permanent colorant, and the third semi-permanent colorant are present at a ratio of about 1:4:1, about 1:3:1, about 1:2:1, about 1:1:1, about 2:1:1, about 1:1:1, about 2:1:1, about 3:1:1, or about 4:1:1 first semi-permanent colorant:second semi-permanent colorant:third semi-permanent colorant. In some embodiments, the first semi-permanent colorant, the second semi-permanent colorant, and the third semi-permanent colorant are present at a ratio of about 1:1:1 first semi-permanent colorant:second semi-permanent colorant:third semi-permanent colorant.

### Composition A

Also provided herein are compositions comprising a semi-permanent colorant (e.g., genipin derivative, lawsone, lawsone derivative, or combinations thereof, e.g., any of the combinations described herein); a solvent; and a semi-volatile semi-permanent colorant solubilizer. In some embodiments, such compositions can be suitable for inkjet printing. The manufacturing approach employing inkjet printing directly onto a skin transfer substrate enables the rapid production of made-to-order custom designed semi-permanent tattoos, but inkjet printing can also impose constraints on the physico-chemical properties of the ink dispensed by the printer nozzles. U.S. application Ser. No. 16/785,549 (U.S. Publication No. 2020/0276101) describes genipin-based compositions that can be suitable for inkjet printing and is incorporated herein by reference in its entirety.

In some embodiments, the semi-permanent colorant (e.g., genipin derivative, lawsone, lawsone derivative, or combinations thereof) is present in an amount of about 0.1% to about 20% w/w of the composition. For example, about 0.1 to about 18%, about 0.1% to about 16%, about 0.1% to about 14%, about 0.1% to about 12%, about 0.1% to about 10%, about 0.1% to about 8%, about 0.1% to about 6%, about 0.1% to about 4%, about 0.1% to about 2%, about 16% to about 18%, about 14% to about 18%, about 12% to about 18%, about 10% to about 18%, about 8% to about 18%, about 6% to about 18%, about 4% to about 18%, or about 2% to about 18% w/w of the composition. In some embodiments, the semi-permanent colorant is present in an amount of about 5% to about 15%, about 5% to about 10%, about 7% to about 11%, about 5% to about 7%, about 6% to about 8%, about 7% to about 9%, about 8% to about 10%, or about 6.5% to about 7.5% w/w of the composition. In some embodiments, the semi-permanent colorant is present in an amount of about 1%, about 2%, about 3%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, or about 20% w/w of the composition. In some embodiments, the semi-permanent colorant comprises a genipin derivative, lawsone, a lawsone derivative, or a combination thereof.

Non-limiting examples of solvents include water, methanol, ethanol, isopropanol, and diethylene glycol monoethyl ether. In some embodiments, the solvent is a volatile solvent, e.g., the solvent can evaporate or vaporize at room temperature. Non-limiting examples of volatile solvents include water, methanol, ethanol, and isopropanol. In some embodiments, the solvent is selected to have a low toxicity profile when used as described herein. For example, the solvent can be selected such that the amount of any residual solvent left after a composition (e.g., a composition described herein) has dried on an applicator is not a toxic amount for contact with a human skin surface.

In some embodiments, the solvent is present in an amount of about 0.1% to about 95% w/w of the composition. For example, about 10% to about 95%, about 20% to about 95%, about 30% to about 95%, about 40% to about 95%, about 50% to about 95%, about 60% to about 95%, about 70% to about 95%, about 80% to about 95%, about 10% to about 20%, about 10% to about 30%, about 10% to about 40%, about 10% to about 50%, about 10% to about 60%, about 10% to about 70%, about 10% to about 80%, or about 10% to about 90% w/w of the composition. In some embodiments, the solvent is present in an amount of about 55% to about 75%, about 60% to about 80%, about 65% to about 85%, about 70% to about 90%, about 75% to about 95%, about 50% to about 75%, about 55% to about 80%, about 60% to about 85%, about 65% to about 90%, about 60% to about 70%, about 65% to about 75%, about 70% to about 80%, or about 75% to about 85% w/w of the composition. For example, about 60% to about 65%, about 62% to about 67%, about 65% to about 70%, about 67% to about 72%, about 70% to about 75%, about 72% to about 77%, about 75% to about 80%, about 77% to about 82%, about 80% to about 85%, about 82% to about 87%, about 85% to about 90%, about 87% to about 92%, or about 90% to about 95% w/w of the composition. In some embodiments, the solvent is present in an amount of about 50%, about 55%, about 60%, about 65%, about 66%, about 67%, about 68%, about 69%, about 70%, about 71%, about 72%, about 73%, about 74%, about 75%, about 76%, about 77%, about 78%, about 79%, about 80%, about 85%, about 90%, or about 95% w/w of the composition.

In some embodiments, the solvent can be a solvent in which the semi-permanent colorant, e.g., a colorant precursor such as genipin, is soluble. For example in some embodiments, the solvent is a solvent in which the semi-permanent colorant, e.g., a colorant precursor such as genipin has a solubility of at least 3% w/w of the composition, for example, at least 4%, at least 5%, at least 6%, at least 7%, at least 8%, or at least 9% w/w of the composition. In some embodiments, the solvent is selected from the group consisting of: water, methanol, ethanol, isopropanol, and a combination thereof.

In some embodiments, a combination of solvents can be used. In some embodiments, each solvent can be independently present in an amount of about 5% to about 80%. For example, about 5% to about 10%, about 5% to about 20%, about 5% to about 30%, about 5% to about 40%, about 5% to about 50%, about 5% to about 60%, about 5% to about 70%, about 70% to about 80%, about 60% to about 80%, about 50% to about 80%, about 40% to about 80%, about 30% to about 80%, about 20% to about 80%, or about 10% to about 80% w/w of the composition. In some embodiments, a solvent can be independently present in an amount of about 20% to about 30%, about 25% to about 35%, about 30% to about 40%, about 35% to about 45%, about 40% to about 50%, or about 45% to about 50% w/w of the composition. For example, about 20%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% w/w of the composition.

In some embodiments, the solvent can include a combination of water and ethanol. In some embodiments, the water can be independently present in an amount of about 40% to about 55% w/w of the composition and the ethanol can be independently present in an amount of about 15% to about 30% w/w of the composition. In some embodiments, the water independently can be present in an amount of about 45% to about 50% w/w of the composition and the independently ethanol can be present in an amount of about 20% to about 27% w/w of the composition. In some embodiments, the water can be independently present in an amount of about 25% to about 40% w/w of the composition and the ethanol can be independently present in an amount of about 35% to about 50% w/w of the composition. In some embodiments, the water can be independently present in an amount of about 25% to about 35% w/w of the composition and the ethanol can be independently present in an amount of about 35% to about 45% w/w of the composition. In some embodiments, the water can be independently present in an amount of about 28% to about 32% w/w of the composition and the ethanol can be independently present in an amount of about 40% to about 45% w/w of the composition.

A “semi-volatile semi-permanent colorant solubilizer” as used herein refers to a semi-volatile solvent in which the semi-permanent colorant (e.g., genipin derivative, lawsone, lawsone derivative, or combination thereof) is soluble. In some embodiments of the compositions described herein, the semi-permanent colorant (e.g., genipin derivative, lawsone, lawsone derivative, or combination thereof) exhibits a solubility of greater than 1 mg/mL in the semi-volatile semi-permanent colorant solubilizer. In some embodiments of the compositions described herein, the semi-permanent colorant (e.g., genipin derivative, lawsone, lawsone derivative, or combination thereof) exhibits a solubility of greater than 5 mg/mL in the semi-volatile semi-permanent colorant solubilizer. Delivery of semi-permanent colorant into the upper skin layers through passive diffusion is driven by the solubility of the semi-permanent colorant in the delivery vehicle and the skin permeability. As such, solvents that enhance the semi-permanent colorant solubility in water can help increase the penetration of the semi-permanent colorant into the stratum corneum. Non-limiting examples of a semi-volatile semi-permanent colorant solubilizer include a polyethylene glycol, an alkyl glycol, and an alkylene glycol ether. An alkylene glycol ether can include ethylene glycol dimethyl ether, ethylene glycol diethyl ether, and ethylene glycol dibutyl ether.

In some embodiments, the semi-volatile semi-permanent colorant solubilizer is present in an amount of about 0.1% to about 95% w/w of the composition. For example, about 1% to about 95%, about 5% to about 95%, about 10% to about 95%, about 20% to about 95%, about 30% to about 95%, about 40% to about 95%, about 50% to about 95%, about 60% to about 95%, about 70% to about 95%, about 80% to about 95%, about 10% to about 20%, about 10% to about 30%, about 10% to about 40%, about 10% to about 50%, about 10% to about 60%, about 10% to about 70%, about 10% to about 80%, or about 10% to about 90% w/w of the composition. In some embodiments, the semi-volatile semi-permanent colorant solubilizer is present in an amount of about 1% to about 30%, about 5% to about 35%, about 10% to about 40%, about 1% to about 20%, about 5% to about 20%, about 10% to about 25%, about 15% to about 30%, about 30% to about 35%, about 25% to about 40%, about 1% to about 10%, about 5% to about 15%, about 10% to about 20%, or about 15% to about 25% w/w of the composition. For example, about 10% to about 15%, about 12% to about 17%, about 15% to about 20%, about 17% to about 22%, about 20% to about 25%, about 22% to about 27%, about 25% to about 30%, about 27% to about 32%, about 30% to about 35%, about 32% to about 37%, or about 35% to about 40% w/w of the composition. In some embodiments, the semi-volatile semi-permanent colorant solubilizer is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w of the composition.

In some embodiments, the semi-volatile semi-permanent colorant solubilizer is selected from the group consisting of: a polyethylene glycol, an alkyl glycol, an alkylene glycol ether, and a combination thereof.

In some embodiments, the semi-volatile semi-permanent colorant solubilizer comprises an alkyl glycol. In some embodiments, the alkyl glycol is diethylene glycol monoethyl ether (e.g., ethoxydiglycol, TRANSCUTOL® CG). In some embodiments, diethylene glycol monoethyl ether is present in an amount of about 1% to about 30%, about 5% to about 35%, about 10% to about 40%, about 1% to about 20%, about 5% to about 20%, about 10% to about 25%, about 15% to about 30%, about 30% to about 35%, about 25% to about 40%, about 1% to about 10%, about 5% to about 15%, about 10% to about 20%, or about 15% to about 25% w/w of the composition. For example, about 10% to about 15%, about 12% to about 17%, about 15% to about 20%, about 17% to about 22%, about 20% to about 25%, about 22% to about 27%, about 25% to about 30%, about 27% to about 32%, about 30% to about 35%, about 32% to about 37%, or about 35% to about 40% w/w of the composition. In some embodiments, diethylene glycol monoethyl ether is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w of the composition. In some embodiments, the semi-volatile semi-permanent colorant solubilizer is diethylene glycol monoethyl ether.

In some embodiments, the composition has a viscosity from about 1 to about 20 centipoise. For example, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about about 1 to about 4, about 1 to about 3, about 1 to about 2, about 19 to about 20, about 18 to about 20, about 17 to about 20, about 16 to about 20, about 15 to about about 14 to about 20, about 13 to about 20, about 12 to about 20, about 11 to about 20, about 10 to about 20, about 9 to about 20, about 8 to about 20, about 7 to about 20, about 6 to about 20, about 5 to about 20, about 4 to about 20, about 3 to about 20 centipoise, or about 2 to about 20 centipoise. In some embodiments, the composition has a viscosity from about 5 to about 15, about 5 to about 10, about 10 to about 15, about 2 to about 4, about 3 to about 5, or about 4 to about 6 centipoise. In some embodiments, the composition has a viscosity of about 3, about 3.25, about 3.5, about 3.75, about 4, about 4.25, about 4.5, about 4.75, about 5, about 5.25, about about 5.75, about 6, about 6.25, about 6.5, about 6.75, about 7, about 8, about 9, about 10, about 11, about 12, about 13, about 14, about 15, about 16, about 17, about 18, about 19, or about 20 centipoise.

In some embodiments, the composition is configured to have a viscosity from about 1 to about 7 centipoise. For example, about 1 to about 6, about 1 to about about 1 to about 4, about 1 to about 3, about 1 to about 2, about 6 to about 7, about 5 to about 7, about 4 to about 7, about 3 to about 7 centipoise, or about 2 to about 7 centipoise. In some embodiments, the composition is configured to have a viscosity from about 2 to about 4, about 3 to about 5, or about 4 to about 6 centipoise. In some embodiments, the composition is configured to have a viscosity of about 3, about 3.25, about 3.5, about 3.75, about 4, about 4.25, about 4.5, about 4.75, about 5, about 5.25, about 5.5, about 5.75, about 6, about 6.25, about 6.5, about 6.75, about 7, about 8, about 9, about 10, about 11, about 12, about 13, about 14, about 15, about 16, about 17, about 18, about 19, or about 20 centipoise.

In some embodiments, the composition has a surface tension from about 10 to about 45 dynes/cm. For example, about 10 to about 20, about 10 to about 30, about 10 to about 45, about 40 to about 45, about 30 to about 45, or about 20 to about dynes/cm. In some embodiments, the composition has a surface tension from about 15 to about 25, about 20 to about 30, about 25 to about 35, about 30 to about about 35 to about 45, or about 20 to about 35 dynes/cm. For example, about 20, about 21, about 22, about 23, about 24, about 25, about 26, about 27, about 28, about 29, about 30, about 31, about 32, about 33, about 34, or about 35 dynes/cm.

In some embodiments, the composition is configured to have a surface tension from about 5 to about 60 dynes/cm. In some embodiments, the composition is configured to have a surface tension from about 10 to about 45 dynes/cm. For example, about 10 to about 20, about 10 to about 30, about 10 to about 45, about 40 to about 45, about 30 to about 45, or about 20 to about 45 dynes/cm. In some embodiments, the composition is configured to have a surface tension from about 15 to about 25, about 20 to about 30, about 25 to about 35, about 30 to about 40, about 35 to about 45, or about 20 to about 35 dynes/cm. For example, about 20, about 21, about 22, about 23, about 24, about 25, about 26, about 27, about 28, about 29, about 30, about 31, about 32, about 33, about 34, or about 35 dynes/cm.

In some embodiments, the composition further comprises a surface-active agent. Non-limiting examples of a surface-active agents include an alkylbenzene sulfonate, an alkyl sulfate, an alkyl ether sulfate, a soap, an ethoxylate, an alkyl alcohol, a lignosulfonate, and a triglyceride. For example, a surface-active agent can include sodium dodecyl benzenesulfonate, lauryl sulfate, di-alkyl sulfosuccinate, dimethyl ether of tetradecyl phosphonic, lauryl mono-ethanol, glycerol diester (diglyceride), dodecyl betaine, abietic acid, polyethoxylated octyl phenol, 1,2-hexanediole (e.g., OPTIPHEN® HD), and sorbitan monoester.

In some embodiments, the surface-active agent is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments, the surface-active agent is present in an amount of about 0.25% to about 5% w/w of the composition. For example, about 0.25% to about 5%, about 0.25% to about 4%, about 0.25% to about 3%, about 0.25% to about 2%, about 0.25% to about 1%, about 2% to about 5%, about 2% to about 4%, about 2% to about 3%, about 1% to about 4%, about 1% to about 3%, about 1% to about 2%, about 3% to about 5%, about 4% to about 5%, about 3% to about 5%, about 2% to about 5%, or about 1% to about 5% w/w of the composition. In some embodiments, the surface-active agent is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition.

In some embodiments, the surface-active agent is selected from the group consisting of: an alkylbenzene sulfonate, a polyethylene glycol, an alkyl sulfate, an alkyl ether sulfate, a soap, an ethoxylate, a lignosulfonate, a triglyceride, and a combination thereof. In some embodiments, the surface-active agent is selected from the group consisting of: sodium dodecyl benzenesulfonate, lauryl sulfate, di-alkyl sulfosuccinate, dimethyl ether of tetradecyl phosphonic, lauryl mono-ethanol, glycerol diester (diglyceride), dodecyl betaine, abietic acid, polyethoxylated octyl phenol, 1,2-hexanediol, sorbitan monoester, and a combination thereof.

In some embodiments, the surface-active agent is 1,2-hexanediol. In some embodiments, 1,2-hexanediol is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments, 1,2-hexanediol is present in an amount of about 0.25% to about 5% w/w of the composition. For example, about 0.25% to about 5%, about 0.25% to about 4%, about 0.25% to about 3%, about 0.25% to about 2%, about 0.25% to about 1%, about 2% to about 5%, about 2% to about 4%, about 2% to about 3%, about 1% to about 4%, about 1% to about 3%, about 1% to about 2%, about 3% to about 5%, about 4% to about 5%, about 3% to about 5%, about 2% to about 5%, or about 1% to about 5% w/w of the composition. In some embodiments, 1,2-hexanediol is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition.

In some embodiments, the surface-active agent is polyethylene glycol 400 (PEG 400). In some embodiments, the PEG 400 is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments, the PEG 400 is present in an amount of about 0.25% to about 5% w/w of the composition. For example, about 0.25% to about 5%, about 0.25% to about 4%, about 0.25% to about 3%, about 0.25% to about 2%, about 0.25% to about 1%, about 2% to about 5%, about 2% to about 4%, about 2% to about 3%, about 1% to about 4%, about 1% to about 3%, about 1% to about 2%, about 3% to about 5%, about 4% to about 5%, about 3% to about 5%, about 2% to about 5%, or about 1% to about 5% w/w of the composition. In some embodiments, the PEG 400 is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition.

In some embodiments, the composition further comprises a matrix component. A matrix component can form a solid or semi-solid, immobilized matrix on a substrate. For example, after a composition as described herein is applied to a substrate and the ink dries, e.g., the volatile solvents have evaporated or nearly evaporated off of the substrate, the matrix component can form a solid or semi-solid, immobilized matrix on a substrate. Non-limiting examples of a matrix component include a sugar, a sugar alcohol (e.g., sorbitol, mannitol, xylitol, isomalt, hydrogenated starch hydrolysates), a polymer, or a combination of two or more thereof. For example, a sugar can include a monosaccharide (e.g., glucose, fructose, galactose, ribose, and xylose), a disaccharide (e.g., trehalose such as TREHALOSE 100, sucrose, lactotrehalose, galactotrehalose, 6-azidotrehalose, maltose, lactose, lactulose, cellobiose, and chitobiose), or an oligosaccharide (e.g., starch, glycogen, cellulose, and chitin). In some embodiments, the polymer can include pullulan, a cellulose derivative (e.g., hydroxypropylmethylcellulose, hydroxyethylcellulose, and hydroxymethylcellulose), an acrylate, polyvinylalcohol, an ethylene oxide/propylene oxide copolymer, and polyester.

In some embodiments, the matrix component is present in an amount of about 0.1% to about 50% w/w of the composition. For example, about 0.1% to about 25%, about 25% to about 50%, or about 15% to about 35% w/w of the composition. In some embodiments, the matrix components is present in an amount of about 0.1% to about 10% w/w of the composition. For example, about 0.1% to about 8%, about 0.1 to about 6%, about 0.1% to about 4%, about 0.1% to about 3%, about 0.1% to about 2%, about 0.1% to about 1%, about 8% to about 10%, about 6% to about 10%, about 4% to about 10%, about 2% to about 10%, about 1% to about 5%, about 1% to about 4%, about 1% to about 3%, about 1% to about 2%, about 2% to about 5%, about 2% to about 4%, about 2% to about 3%, or about 3% to about 5% w/w of the composition. In some embodiments, the matrix component is present in an amount of about 0.1%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the matrix component is selected from the group consisting of: a sugar, a sugar alcohol, a polymer, or a combination thereof. In some embodiments, the matrix component is selected from the group consisting of: sorbitol, mannitol, xylitol, isomalt, hydrogenated starch hydrolysates, glucose, fructose, galactose, ribose, xylose, trehalose, sucrose, lactotrehalose, galactotrehalose, 6-azidotrehalose, maltose, lactose, lactulose, cellobiose, chitobiose, starch, glycogen, cellulose, chitin, pullulan, hydroxypropylmethylcellulose, hydroxyethylcellulose, hydroxymethylcellulose, an acrylate, polyvinylalcohol, an ethylene oxide/propylene oxide copolymer, polyester, and a combination thereof.

In some embodiments, the matrix component is trehalose. In some embodiments, trehalose is present in an amount of about 0.1% to about 8%, about 0.1% to about 6%, about 0.1% to about 4%, about 0.1% to about 3%, about 0.1% to about 2%, about 0.1% to about 1%, about 8% to about 10%, about 6% to about 10%, about 4% to about 10%, about 2% to about 10%, about 1% to about 5%, about 1% to about 4%, about 1% to about 3%, about 1% to about 2%, about 2% to about 5%, about 2% to about 4%, about 2% to about 3%, or about 3% to about 5% w/w of the composition. In some embodiments, trehalose is present in an amount of about 0.1%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the matrix component is hydroxypropylmethylcellulose. In some embodiments, hydroxypropylmethylcellulose is present in an amount of about 0.1% to about 8%, about 0.1% to about 6%, about 0.1% to about 4%, about 0.1% to about 3%, about 0.1% to about 2%, about 0.1% to about 1%, about 8% to about 10%, about 6% to about 10%, about 4% to about 10%, about 2% to about 10%, about 1% to about 5%, about 1% to about 4%, about 1% to about 3%, about 1% to about 2%, about 2% to about 5%, about 2% to about 4%, or about 3% to about 5% w/w of the composition. In some embodiments, hydroxypropylmethylcellulose is present in an amount of about 0.1%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the composition further comprises a skin penetration enhancer. “Skin penetration enhancer” as used herein refers to a substance that penetrates (penetrant) into skin to reversibly decrease the barrier resistance. In some embodiments, a skin penetration enhancer can also enhance the solubility of the penetrant to increase loading, which may, for example, enhance the flux of the penetrant across the skin. Non-limiting examples of a skin penetration enhancer include an alcohol, an amide, an ester, an ether alcohol, a fatty acid, a glycol, a pyrrolidone, a sulphoxide, a surfactant, and a terpene. For example, a skin penetration enhancer can include ethanol, isopropanol, decanol, hexanol, lauryl alcohol, myristyl alcohol, octanol, octyl dodecanol, oleyl alcohol, azone, ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, propylene glycol monocaprylate, diethylene glycol monoethyl ether (TRANSCUTOL®), lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, and neridol. See, for example, Lane, Int. J. Pharm. 447:12-21, 2013, which is herein incorporated by reference in its entirety.

In some embodiments, the skin penetration enhancer is present in an amount of about 0.1% to about 50% w/w of the composition. For example, about 0.1% to about 25%, about 25% to about 50%, or about 15% to about 35% w/w of the composition. In some embodiments, the skin penetration enhancer is present in an amount of about 0.1% to about 10% w/w of the composition. In some embodiments, the skin penetration enhancers is present in an amount of about 0.5% to about 5% w/w of the composition. For example, about 0.5% to about 5%, about 0.5% to about 4%, about 0.5% to about 3%, about 0.5% to about 2%, about 2% to about 5%, about 2% to about 4%, or about 3% to about 5% w/w of the composition. In some embodiments, the skin penetration enhancer is present in an amount of about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the skin penetration enhancer is selected from the group consisting of: an alcohol, an amide, an ester, an ether alcohol, a fatty acid, a glycol, a pyrrolidone, a sulphoxide, a surfactant, a terpene, and a combination thereof. In some embodiments, the skin penetration enhancer is selected from the group consisting of: ethanol, isopropanol, decanol, hexanol, lauryl alcohol, myristyl alcohol, octanol, octyl dodecanol, oleyl alcohol, azone, ethyl acetate, octyl salicylate, padimate ethyl oleate, glyceryl monoleate, glyceryl monocaprate, glyceryl tricaprylate, isopropyl myristate, isopropyl palmitate, propylene glycol monolaurate, propylene glycol monocaprylate, diethylene glycol monoethyl ether (TRANSCUTOL®), lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, neridol, and a combination thereof.

In some embodiments, the skin penetration enhancer is selected from the group consisting of: an alcohol, an amide, an ester, an ether alcohol, a fatty acid, a glycol, a pyrrolidone, a sulphoxide, a surfactant, a terpene, and a combination thereof. In some embodiments, the skin penetration enhancer is selected from the group consisting of: ethanol, isopropanol, decanol, hexanol, lauryl alcohol, myristyl alcohol, octanol, octyl dodecanol, oleyl alcohol, azone, ethyl acetate, octyl salicylate, padimate ethyl oleate, glyceryl monoleate, glyceryl monocaprate, glyceryl tricaprylate, isopropyl myristate, isopropyl palmitate, propylene glycol monolaurate, propylene glycol monocaprylate, lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, neridol, and a combination thereof.

In some embodiments, the composition further comprises a temporary colorant. In some embodiments, the temporary colorant can be used to show what the semi-permanent tattoo will look like, outline the location of the semi-permanent tattoo on an applicator, provide a logo, e.g., a brand logo, that does not transfer to the skin or transfers but is present for a short time as compared to the duration of the semi-permanent tattoo. In some embodiments, the temporary colorant can be used to provide a changing semi-permanent tattoo that changes appearance with time, e.g., with semi-permanent colorant duration and temporary colorant duration. Non-limiting examples of temporary colorants include iron oxide black (Fe₃O4), iron oxide (FeO), carbon, logwood, ochre, cinnabar (HgS), cadmium red (CdSe), iron (III) oxide (Fe₂O₃), naphthol-AS pigment, disazodiarylide, disazopyrazolone, cadmium seleno-sulfide, cadmium yellow (CdS, CdZnS), curcuma yellow, chrome yellow (PbCrO₄), disazodiarylide, chromium oxide (Cr₂O₃), malachite [Cu₂(CO₃)(OH)₂], a ferrocyanide, a ferricyanide, lead chromate, monoazo pigment, Cu/Al phthalocyanine, Cu phthalocyanine, azure blue, cobalt blue, Cu-phthalocyanine, manganese violet (manganese ammonium pyrophosphate), an aluminum salt, quinacridone, dioxazine/carbazole, lead white (lead carbonate), titanium dioxide (TiO₂), barium sulfate (BaSO₄), zinc oxide, anthraquinone dyes and derivatives, annatto, caramel, 11-carotene, bismuth citrate, disodium EDTA copper, potassium sodium copper chlorophyllin, dihydroxyacetone, bismuth oxychloride, guaiazulene, henna, ferric ammonium ferrocyanide, ferric ferrocyanide, chromium hydroxide green, chromium oxide greens, guanine, lead acetate, pyrophillite, mica, silver, titanium dioxide, aluminum powder, bronze powder, copper powder, ultramarines, manganese violet, zinc oxide, luminescent zinc sulfide, D&C Black Nos. 2 and 3, FD&C Blue No. 1 (e.g., acid blue 9), D&C Blue No. 4, D&C Brown No. 1, FD&C Green No. 3, D&C Green Nos. 6, and 8, D&C Orange Nos. 4, 5, 10 and 11, FD&C Red Nos. 4, D&C Red Nos. 6, 7, 17, 21, 22, 27, 28, 30, 32, 33, 34, 36 and 40, D&C Violet No. 2, Ext. D&C Violet No. 2, FD&C Yellow Nos. 5 and 6, D&C Yellow Nos. 7, 8, 10 and 11, and Ext. D&C Yellow No. 7. In some embodiments, the temporary colorant does not contain an amine group.

In some embodiments, the temporary colorant is present in an amount of about 0.01% to about 5% w/w of the composition. For example, about 0.01% to about 5%, about 0.01% to about 4%, about 0.01% to about 3%, about 0.01% to about 2%, about 0.01% to about 1%, about 0.01% to about 0.5%, about 0.5% to about 5%, about 1% to about 5%, about 2% to about 5%, about 3% to about 5% w/w, about 4% to about 5%, or about 2% to about 4% w/w of the composition. In some embodiments, the temporary colorant is present in an amount of about 0.06% to about 0.1%, about 0.07% to about 0.11%, about 0.08% to about 0.12%, about 0.09% to about 0.13%, about 0.1% to about 0.14%, about 0.11% to about 0.15%, about 0.12% to about 0.16% about 0.13% to about 0.17%, about 0.14% to about 0.18%, about 0.15% to about 0.19%, about 0.16% to about 0.2%, about 0.17% to about 0.21%, or about 0.18% to about 0.22% w/w of the composition. In some embodiments, the temporary colorant is present in an amount of about 0.01%, about 0.02%, about 0.03%, about 0.04% about 0.05%, about 0.06%, about 0.07%, about 0.08%, about 0.09%, about about 0.11%, about 0.12%, about 0.13%, about 0.14%, about 0.15%, about about 0.17%, about 0.18%, about 0.19%, about 0.2%, about 0.3%, about about 0.5%, about 0.6%, about 0.8%, about 1% to about 2%, about 1.5% to about 2.5%, about 2% to about 3%, about 2.5% to about 3%, about 3% to about 4%, about 3.5% to about 4.5%, or about 4% to about 5% w/w of the composition. In some embodiments, the temporary colorant is present in an amount of about 0.01% to about 1%, about 0.01% to about 0.1%, about 0.01% to about 0.2%, about 0.01% to about about 0.01% to about 0.4%, about 0.01% to about 0.5%, about 0.01% to about about 0.01% to about 0.7%, about 0.01% to about 0.8%, about 0.01% to about 0.9%, about 0.9% to about 1%, about 0.8% to about 1%, about 0.7% to about 1%, about 0.6% to about 1%, about 0.5% to about 1%, about 0.4% to about 1%, about 0.3% to about 1%, about 0.2% to about 1%, about 0.1% to about 1% w/w of the composition. For example, about 0.1% to about 0.3%, about 0.2% to about 0.4%, or about 0.3% to about 0.5% w/w of the composition. In some embodiments, the temporary colorant is present in an amount of about 0.01%, about 0.05%, about 0.1%, about 0.15%, about 0.2%, about 0.25%, about 0.3%, about 0.35%, about 0.4%, about about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the temporary colorant is acid blue 9 (e.g., FD&C Blue No. 1). In some embodiments, acid blue 9 is present in an amount of about 0.01% to about 5% w/w of the composition. For example, about 0.01% to about 5%, about 0.01% to about 4%, about 0.01% to about 3%, about 0.01% to about 2%, about 0.01% to about 1%, about 0.01% to about 0.5%, about 2% to about 5%, about 3% to about 5% w/w, about 4% to about 5%, or about 2% to about 4% of the composition. In some embodiments, acid blue 9 is present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.3%, about 0.4%%, about 0.5%, about 0.6%, about 0.8%, about 1% to about 2%, about 1.5% to about 2.5%, about 2% to about 3%, about 2.5% to about 3%, about 3% to about 4%, about 3.5% to about 4.5%, or about 4% to about 5% w/w of the composition. In some embodiments, acid blue 9 is present in an amount of about about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the composition further comprises a preservative. “Preservative” as used herein refers to an agent that protects against decay, discoloration, and/or spoilage. Non-limiting examples of a preservative include ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea]polyaminopropyl biguanide, methenamine, quaternium-climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), sodium lignosulfonate, butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, and a tocopherol.

In some embodiments, the preservative is present in an amount of about 0.05% to about 5% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 4% to about 5%, about 3% to about 5%, about 2% to about 5%, about 1% to about 5%, about 0.5% to about 5%, or about 0.1% to about 5% w/w of the composition. In some embodiments, the preservative is present in an amount of 0.1% to about 2%, 0.5% to about 2.5%, about 1% to about 3%, about 1.5% to about 3.5%, about 2% to about 4%, about 2.5% to about 4.5% w/w of the composition. In some embodiments, the preservative is present in an amount of about 0.5% to about 2% w/w of the composition. In some embodiments, the preservative is present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the preservative is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition.

In some embodiments, the preservative is selected form the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), sodium lignosulfonate, butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, a tocopherol, and a combination thereof.

In some embodiments, the preservative is a combination of phenoxyethanol and ethylhexylglycerin (e.g., Euxyl® PE9010). In some embodiments, the combination of phenoxyethanol and ethylhexylglycerin is present in the composition at a ratio of 9:1 phenoxyethanol:ethylhexylglycerin.

In some embodiments, the combination of phenoxyethanol and ethylhexylglycerin is present in an amount of about 0.05% to about 5% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 4% to about 5%, about 3% to about 5%, about 2% to about 5%, about 1% to about 5%, about 0.5% to about 5%, or about 0.1% to about 5% w/w of the composition. In some embodiments, the combination of phenoxyethanol and ethylhexylglycerin is present in an amount of 0.1% to about 2%, 0.5% to about 2.5%, about 1% to about 3%, about 1.5% to about 3.5%, about 2% to about 4%, about 2.5% to about 4.5% w/w of the composition. In some embodiments, the combination of phenoxyethanol and ethylhexylglycerin is present in an amount of about 0.5% to about 2% w/w of the composition. In some embodiments, the combination of phenoxyethanol and ethylhexylglycerin is present in an amount of about 0.5% to about 1.5%, about 0.5% to about 1.0%, about 0.05% to about 0.5%, about 0.05% to about 0.1%, about 1.5% to about 2%, about 1% to about 2%, about 0.5% to about 2%, or about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the combination of phenoxyethanol and ethylhexylglycerin is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition.

In some embodiments, the composition is adjusted to a pH of about 4 to about 7. For example, about 4 to about 5, about 4.5 to about 5.5, about 5 to about 6, about 5.5 to about 6.5, or about 6 to about 7. In some embodiments, the composition is adjusted to a pH of about 4, about 4.25, about 4.5, about 4.75, about 5, about 5.25, about 5.5, about 5.75, about 6, about 6.25, about 6.5, about 6.75, or about 7. In some embodiments, the composition is adjusted to a pH of about 4 to about 6. In some embodiments, the composition is adjusted to a pH of about 4.6.

In some embodiments, the composition comprises:
a semi-permanent colorant (e.g., a genipin derivative, lawsone, a lawsone derivative, or a combination thereof, e.g., any of the combinations described herein) present in an amount of about 5% to about 10% w/w of the composition;
a solvent present in an amount of about 65% to about 80% w/w of the composition;
a semi-volatile semi-permanent colorant solubilizer present in an amount of about 5% to about 25% w/w of the composition. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 7% w/w of the composition;
a solvent present in an amount of about 65% to about 80% w/w of the composition;
a semi-volatile semi-permanent colorant solubilizer present in an amount of about 5% to about 25% w/w of the composition. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 7% w/w of the composition;
a solvent present in an amount of about 70% to about 75% w/w of the composition;
a semi-volatile semi-permanent colorant solubilizer present in an amount of about 15% w/w of the composition. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 7% w/w of the composition;
a solvent present in an amount of about 73% w/w of the composition;
a semi-volatile semi-permanent colorant solubilizer present in an amount of about 15% w/w of the composition. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 5% to about 10% w/w of the composition;
a solvent present in an amount of about 65% to about 80% w/w of the composition, wherein the one or more semi-volatile solvents are water and ethanol;
a semi-volatile semi-permanent colorant solubilizer present in an amount of about 5% to about 25% w/w of the composition, wherein the semi-volatile semi-permanent colorant solubilizer comprises diethylene glycol monoethyl ether. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 7% w/w of the composition;
a solvent present in an amount of about 70% to about 75% w/w of the composition, wherein the one or more semi-volatile solvents are water and ethanol;
a semi-volatile semi-permanent colorant solubilizer present in an amount of about 5% to about 25% w/w of the composition, wherein the semi-volatile semi-permanent colorant solubilizer comprises diethylene glycol monoethyl ether. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 7% w/w of the composition;
a volatile solvent present in an amount of about 70% to about 75% w/w of the composition, wherein the one or more semi-volatile solvents are water and ethanol;
a semi-volatile semi-permanent colorant solubilizer present in an amount of about 15% w/w of the composition, wherein the semi-volatile semi-permanent colorant solubilizer comprises diethylene glycol monoethyl ether. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the composition comprises:
a semi-permanent colorant (e.g., a genipin derivative, lawsone, a lawsone derivative, or a combination thereof, e.g., any of the combinations described herein) present in an amount of about 7% w/w of the composition;
a volatile solvent present in an amount of about 73% w/w of the composition, wherein the one or more semi-volatile solvents are water and ethanol;
a semi-volatile semi-permanent colorant solubilizer present in an amount of about 15% w/w of the composition, wherein the semi-volatile semi-permanent colorant solubilizer comprises diethylene glycol monoethyl ether. In some embodiments, the composition further comprises a surface-active agent present in an amount of about 1% to about 3% w/w of the composition. In some embodiments, the composition further comprises a surface-active agent present in an amount of about 2% w/w of the composition. In some embodiments, the composition further comprises a matrix component present in an amount of about 1% to about 3% w/w of the composition. In some embodiments, the composition further comprises a matrix component present in an amount of about 1% to about 2% w/w of the composition. In some embodiments, the composition further comprises a preservative present in an amount of about 0.1% to about 2% w/w of the composition. In some embodiments, the composition further comprises a preservative present in an amount of about 1% w/w of the composition. In some embodiments, the composition further comprises a temporary colorant present in an amount of about 0.1% to about 0.5% w/w of the composition. In some embodiments, the composition further comprises a temporary colorant present in an amount of about 0.3% w/w of the composition.

In some embodiments, the surface-active agent is 1,2-hexanediol. In some embodiments, the matrix component is trehalose. In some embodiments, the preservative is a combination of phenoxyethanol and ethylhexylglycerin (e.g., Euxyl® PE9010). In some embodiments, the combination of phenoxyethanol and ethylhexylglycerin are present in the composition at a ratio of 9:1 phenoxyethanol:ethylhexylglycerin. In some embodiments, the temporary colorant is acid blue 9.

In some embodiments, the viscosity of the composition is about 3 to about 6 centipoise. In some embodiments, the viscosity of the composition is about 3.7 to about 4.1 centipoise. In some embodiments, the viscosity of the composition is about 3.8 centipoise.

In some embodiments, the dynamic surface tension of the composition is about 25 to about 30 dynes/cm. In some embodiments, the dynamic surface tension of the composition is about 25 to about 26 dynes/cm. In some embodiments, the dynamic surface tension of the composition is about 26 dynes/cm.

Also provided herein are applicator articles comprising a composition (e.g., Composition A as described in any of the above embodiments); an adhesive; and an adhesive backing.

In some embodiments, an adhesive applicator article described herein can further comprise a non-adhesive liner.

In some embodiments, an adhesive applicator article can further comprise, or can be configured for use with, a heat pad.

In some embodiments of an adhesive applicator article described herein, a composition as described in any of the above embodiments (e.g., Composition A) is disposed on and/or coupled to at least a portion of the adhesive. In some embodiments of an adhesive applicator article described herein, the composition is dried on the applicator article. For example, after the composition is printed onto the adhesive, it can be dried by heating. In some embodiments, the material can be heated and/or dried using an oven, a dual convection oven, a conveyor, and/or an infrared oven. In some embodiments, the composition is dried at about 40° C. to about 80° C. For example, about 40° C. to about 50° C., about 45° C. to about 55° C., about 50° C. to about 60° C., about 55° C. to about 65° C., about 60° C. to about 70° C., about 65° C. to about 75° C., or about 70° C. to about 80° C. In some embodiments, the composition is dried at about 50° C., about 55° C., about 60° C., about 65° C., about 70° C., or about 75° C. In some embodiments, the composition is dried for a period of time. For example, the composition can be dried for about 0.25 to about 3 hours. In some embodiments, the composition is dried for about 0.25 hours to about 0.75 hours, about 0.5 hours to about 1 hour, about 0.75 hours to about 1.25 hours, about 1 hour to about 1.5 hours, about 1.25 hours to about 1.75 hours, about 1.5 hours to about 2 hours, about 1.75 hours to about 2.25 hours, about 2 hours to about 2.5 hours, about 2.25 hours to about 2.75 hours, or about 2.5 hours to about 3 hours. In some embodiments, the composition is dried for about 0.5 hours, about 1 hour, about 1.5 hours, about 2 hours, about 2.5 hours, or about 3 hours. In some embodiments, the composition is dried for about 1.25 hours to about 1.75 hours at about 55° C. to about 65° C. In some embodiments, the composition is dried for about 1.5 hours at about 60° C. In some embodiments, the composition is dried for about 1 hour at about 60° C.

In some embodiments, an adhesive applicator article as described herein can further comprise a medicament or cosmetic for delivery to a subject. In some embodiments, the adhesive applicator article can comprise a patch for delivering a medicament or a cosmetic. In some embodiments, the composition is printed on a patch.

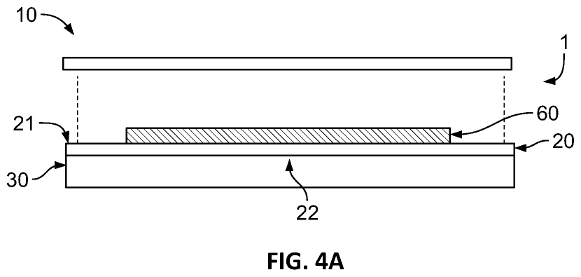

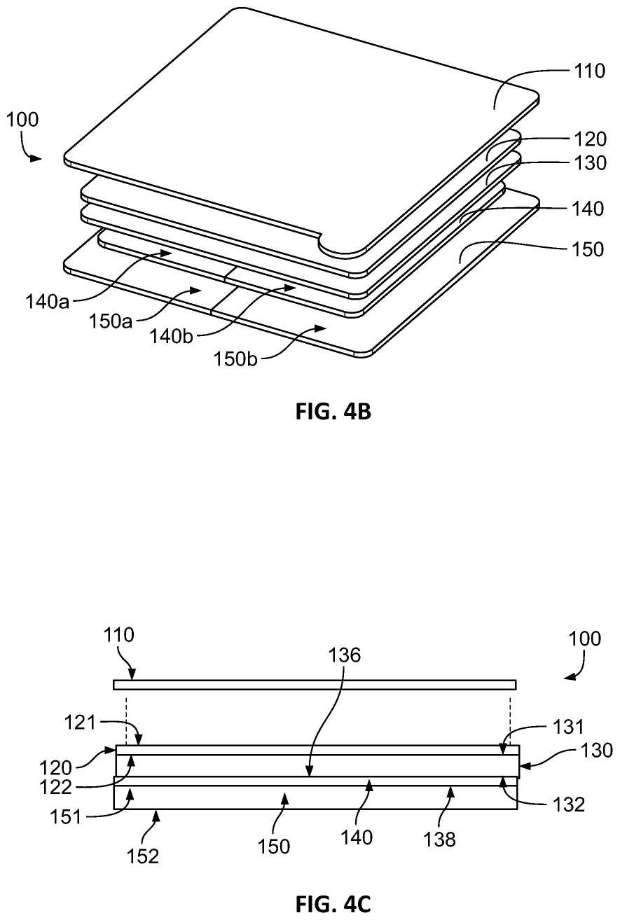

FIG. 4A illustrates an adhesive applicator article 1 according to one embodiment of the present disclosure. As shown in FIG. 4A, in some embodiments, the adhesive applicator article comprises an adhesive layer 20; an adhesive backing 30; and a composition 60 for example, a composition as described in the above embodiments (e.g., Composition A). In some embodiments, the adhesive applicator article optionally further comprises a liner 10. Liner 10 can be, e.g., a protective liner and/or a nonstick liner.

The adhesive layer 20 can include a first surface 21 and a second surface 22 opposite the first surface. In the illustrated embodiment, the adhesive layer 20 is shown with its second surface 22 disposed on and/or coupled to a first surface of the adhesive backing 30. The first surface 21 and the second surface 22 of the adhesive layer 20 are described further herein in connection with FIG. 4C, additionally, the first surface and a second surface of the adhesive backing 30 are described further herein in connection with FIG. 4C.

The composition 60 as described above is deposited on and/or coupled to the first surface 21 of the adhesive layer 20. In some embodiments, the composition 60 is deposited on and/or coupled to a portion of the first surface 21 of the adhesive layer 20. In some embodiments, the composition 60 is printed, e.g., via inkjet printing, onto the first surface 21 of the adhesive layer 20. In some embodiments, wherein the composition 60 is deposited on and/or coupled to a portion of the first surface 21 of the adhesive layer 20, the composition 60 is printed onto the first surface 21 of the adhesive layer 20 in any number of designs. For example, the composition 60 can be printed as a pattern, a shape, words, a picture, an image or art created by a user, or combinations of two or more thereof. In some embodiments, multiple layers of the composition 60 can be printed onto the first surface 21 of the adhesive layer 20 to create a desired pattern and/or effect.

In some optional embodiments, wherein the adhesive applicator article further comprises a liner 10, the liner 10 is deposited on and/or coupled to the composition 60. In some embodiments, wherein the adhesive applicator article further comprises a liner 10, the liner 10 is deposited on and/or coupled to the composition 60 and the first surface 21 of the adhesive layer 20, wherein the composition 60 is deposited on and/or coupled to a portion of the first surface 21 of the adhesive layer 20. In some embodiments, the liner 10 is a non-adhesive liner.

In some embodiments, the adhesive applicator article 1 is configured to provide proper structure for printing, manufacturing, storage, and/or application of a composition comprising a semi-permanent colorant such as those described herein (e.g., Composition A). In some embodiments, the adhesive applicator article 1 is configured to provide proper structure to eliminate or reduce the risk of the adhesive folding in on itself, e.g., during application, and/or to improve the ease of application of the applicator article to a surface, e.g., skin surface, by a user.

In some embodiments, the adhesive applicator article 1 optionally further comprises a heat pad. In some embodiments, the heat pad is adhered to the second surface of the adhesive backing 30. In some embodiments, the heat pad is not attached to the adhesive applicator article 1 and is placed by a user onto the second surface of the adhesive backing 30 during application of the composition 60 to skin.

FIG. 4B illustrates an exploded perspective view of adhesive applicator article 100 according to another embodiment of the present disclosure. FIG. 4C illustrates a side view of the adhesive applicator article 100. As shown in FIG. 4B, in some embodiments, the adhesive applicator article 100 comprises a first adhesive layer 120; a first backing 130 (which can be analogous to the adhesive backing 30 described in connection with FIG. 4A); a second adhesive layer 140, and a second backing 150. In some embodiments, the adhesive applicator article 100 optionally further comprises a liner 110. In some embodiments, the adhesive applicator article 100 optionally further comprises a composition, e.g., Composition A as described above.

As illustrated by FIG. 4C, the first adhesive layer 120 can include a first surface 121 and a second surface 122 opposite the first surface 121. The first adhesive layer 120 is shown with its second surface 122 as disposed on and/or coupled to the first surface 131 of the first backing 130. The second surface 132 of the first backing 130 can be disposed on and/or coupled to the first surface 136 of the second adhesive layer 140. A second surface 138 of the second adhesive layer 140 is shown as disposed on and/or coupled to the first surface 151 of the second backing 150, where the first surface 151 of the second backing 150 is opposite the second surface 152 of the second backing 150.

In some embodiments, the first adhesive layer 120 can optionally have a backing disposed on and/or coupled to its second surface 122. In some embodiments, the first backing 130 can optionally have an adhesive layer disposed on and/or coupled to its first surface 131, its second surface 132, or both. In some embodiments, wherein the first backing 130 has an adhesive layer disposed on and/or coupled to its first surface 131, and its second surface 132, the adhesive layer disposed on and/or coupled to its first surface 131 can be a stronger adhesive than adhesive layer disposed on and/or coupled to its second surface 132. For example, in some embodiments, the weaker peel strength is adhered to the first adhesive layer 120 and the stronger adhesive is adhered to the second backing 150 such that the second backing 150 is easily removed without leaving a residue. In some embodiments, the second adhesive layer 140 can optionally have a backing disposed on and/or coupled to its first surface 136, its second surface 138, or both, as depicted in FIG. 4C. In some embodiments, the second backing 150 can optionally have an adhesive layer disposed on and/or coupled to the first surface 151. In some embodiments, wherein the second backing 150 has an adhesive layer disposed on and/or coupled to a first surface 151, and a second surface 152, the second adhesive layer 140 disposed on and/or coupled to its first surface 151 can be a stronger adhesive than an adhesive layer disposed on and/or coupled to its second surface 152. In some embodiments, wherein the second backing 150 has an adhesive layer disposed on and/or coupled to its first surface 151, and its second surface 152, a liner can be disposed on and/or coupled to the second surface 152 of the second backing 150.

In some embodiments, a composition, e.g., Composition A as described above, can be deposited on and/or coupled to at least a portion of the first surface 121 of the first adhesive layer 120. In some embodiments, the composition can be deposited on and/or coupled to a portion of the first surface 121 of the first adhesive layer 120. In some embodiments, the composition is printed onto the first surface 121 of the first adhesive layer 120. In some embodiments, the composition can be printed onto the first surface 121 of the first adhesive layer 120 using an inkjet printer. In some embodiments, wherein the composition is deposited on and/or coupled to a portion of the first surface 121 of the first adhesive layer 120, the composition can be printed onto the first surface 121 of the first adhesive layer 120 in any number of designs. For example, the composition can be printed as a pattern, a shape, words, a picture, an image or art created by a user, or combinations of two or more thereof. Although examples of a pattern, a word, a picture, an image, and/or art created by a user are mentioned herein, embodiments are not so limited. For example, solid gradients of shapes (e.g., substantially solid ink distribution), variant gradients of shapes, various thicknesses of lines, among other effects can be printed. In some embodiments, multiple layers of the composition can be printed onto the first surface 121 of the first adhesive layer 120 to create a desired pattern and/or effect.

In some embodiments, the second adhesive 140 and second backing 150 are disposed on and/or coupled to at least a portion of the second surface 132 of the first backing 130. In some embodiments, a portion of a surface can include a perimeter, a portion of a perimeter, a grid, a section, and the like.

As illustrated in FIG. 4B, in some embodiments, each layer can optionally be composed of multiple parts or portions. For example, the second adhesive 140 and second backing 150 can be divided into two or more parts, e.g., part 140a and part 140b of the second adhesive 140, and part 150a and part 150b of the second backing 150. In some embodiments, the two or more parts can have an overlapping arrangement, can abut each other, can be separated by a distance, or a combination thereof. A portion can include a perimeter, a portion of a perimeter, a grid, a section, and the like.

In some embodiments, wherein the applicator article 100 further comprises a liner 110 and a composition (e.g., the composition 60 as described above in connection with FIG. 4A), the liner 110 can be disposed on and/or coupled to the composition. In some embodiments, wherein the adhesive applicator article 100 further comprises a liner 110 and a composition as described above (e.g., Composition A), the liner 110 can be disposed on and/or coupled to the composition and the first surface 121 of the first adhesive layer 120, wherein the composition is deposited on and/or coupled to a portion of the first surface 121 of the first adhesive layer 120. In some embodiments, the liner 110 is a non-adhesive liner. In some embodiments, the liner 110 can be silicone coated, fluorosilicone coated, wax coated, coated with a non-stick polymer, or similarly coated with a non-stick layer. In some embodiments, the liner 110 can be a silicone-coated paper liner.

In some embodiments, the first adhesive layer 120 can comprise an acrylic adhesive, a rubber adhesive, or other appropriate adhesive configured to receive the composition, allow it to dry, and allow release and penetration of at least a portion of the composition onto and/or into the skin of a user to which it is applied.

In some embodiments, the first backing 130 can be a polyurethane backing or other appropriate backing that is configured to provide, e.g., desired structure, strength, or geometry to the adhesive applicator article. In some embodiments, the first backing 130 can reduce and/or eliminate the stickiness of an adjacent adhesive layer. For example, the first backing 130 can reduce and/or eliminate the stickiness of the first adhesive layer 120. In some embodiments, the first backing 130 can increase the stiffness of the adhesive applicator article 100 during fabrication and/or use.

In some embodiments, the second adhesive layer 140 can be a laminating adhesive.

In some embodiments, the second backing 150 can be a polycarbonate backing that is configured to provide, e.g., desired structure, strength, or geometry to the adhesive applicator article 100. In some embodiments, the second backing 150 can provide structure that inhibits or prevents wrinkling of the adhesive during the application period.

In some embodiments, the adhesive applicator article 100 is configured to provide proper structure for printing, manufacturing, storage, and/or application of a composition comprising a semi-permanent colorant such as those described herein (e.g., Composition A).

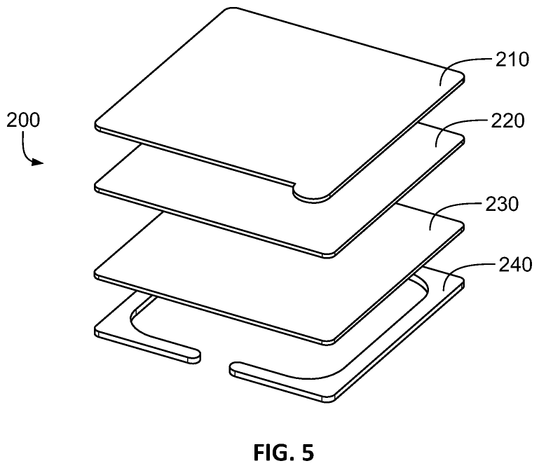

FIG. 5 illustrates an adhesive applicator article 200 according to another embodiment of the present disclosure. As shown in FIG. 5, in some embodiments, the adhesive applicator article 200 comprises an adhesive layer 220; a backing 230; and a structural layer 240. In some embodiments, the adhesive applicator article 200 optionally further comprises a liner 210. In some embodiments, the adhesive applicator article 200 optionally further comprises a composition (e.g., the composition as described above in connection with FIG. 4A). The adhesive layer 220 can, in some embodiments, be analogous to the adhesive layer 20, the adhesive layer 120, and/or the second adhesive layer 140, as described respectively in connection with FIGS. 4A, 4B, and 4C. The backing 230 can, in some embodiments, be analogous to the adhesive backing 30, the backing 130, and/or the second backing 150 as described respectively in connection with FIGS. 4A, 4B, and 4C. The liner 210 can, in some embodiments, be analogous to the liner 10, and/or the liner 110 as described respectively in connection with FIGS. 4A, 4B, and 4C.

Although not specifically labeled in FIG. 5, as to not obstruct the embodiments of the disclosure, the adhesive layer 220 can include a first surface and a second surface opposite the first surface as described in connection with FIG. 4C. Although not specifically labeled in FIG. 5, as to not obstruct the embodiments of the disclosure, the backing 230 can include a first surface and a second surface opposite the first surface as described in connection with FIG. 4C. The adhesive layer 220 is shown with its second surface as disposed on and/or coupled to a first surface of the backing 230. A second surface of the backing 230 is disposed on and/or coupled to a first surface of the structural layer 240.

In some embodiments, the adhesive layer 220 can optionally have a backing disposed on and/or coupled to its second surface. In some embodiments, the backing 230 can optionally have an adhesive layer disposed on and/or coupled to its first surface, its second surface, or both. In some embodiments, the structural layer 240 can optionally have an adhesive layer disposed on and/or coupled to its first surface, its second surface, or both.

In some embodiments, the backing 230 is laminated onto the adhesive layer 220.

In some embodiments, a composition (e.g., the composition 60 as described above in connection with FIG. 4A) is deposited on and/or coupled to at least a portion of the first surface of the adhesive layer 220. In some embodiments, the composition is deposited on and/or coupled to a portion of the first surface of the adhesive layer 220. In some embodiments, the composition is printed onto the first surface of the adhesive layer 220. In some embodiments, the composition can be printed onto the first surface of the adhesive layer 220 using an inkjet printer. In some embodiments, wherein the composition is deposited on and/or coupled to a portion of the first surface of the adhesive layer 220, the composition is printed onto the first surface of the adhesive layer 220 in any number of designs. For example, the composition can be printed as a pattern, a shape, words, a picture, an image or art created by a user, or combinations of two or more thereof. In some embodiments, multiple layers of the composition can be printed onto the first surface of the adhesive layer 220 to create a desired pattern and/or effect.

In some embodiments, the structural layer 240 is disposed on and/or coupled to a portion of the backing 230. In some embodiments, the liner 210 functions as an additional structural layer. For example, the liner 210 can be a framing. In some embodiments, a portion of a surface can include a perimeter, a portion of a perimeter, and a grid, a section, and the like. In some embodiments, the structural layer 240 and/or the liner 210) can be kiss cut. For example, die cut wherein the die only penetrates the structural layer. In some embodiments, the inner corners of the structural layer 240 are rounded. Rounded corners may, for example, prevent tearing during removal and/or provide an indication to the user about where to remove the applicator.

In some embodiments, one or more of the layers of the adhesive article 200 can be composed of more than one part or portion. In some embodiments, the two or more parts can have an overlapping arrangement, can abut each other, or the two or more parts can be a combination thereof. In some embodiments, the two or more parts are a portion of an adjacent layer. A portion can include a perimeter, a portion of a perimeter, and a grid.

In some embodiments, wherein the adhesive applicator article 200 optionally further comprises a liner 210 and a composition (e.g., a composition as described above in connection with FIG. 4A), the liner 210 is disposed on and/or coupled to the composition 260. In some embodiments, wherein the adhesive applicator article further comprises a liner 210 and a composition as described above 260, the liner 210 is disposed on and/or coupled to the composition 260 and the first surface of the adhesive layer 220, wherein the composition is deposited on and/or coupled to a portion of the first surface of the adhesive layer 220. In some embodiments, the liner 210 is a non-adhesive liner. For example, in some embodiments, the liner 210 is can be silicone coated, fluorosilicone coated, wax coated, coated with a non-stick polymer, or similarly coated with a non-stick layer. In some embodiments, the liner 210 is can be a silicone-coated paper liner. In some embodiments, the liner 210 is can be a fluorosilicone-coated paper liner.

In some embodiments, the adhesive layer 220 can be an acrylic adhesive, a rubber adhesive, or other appropriate adhesive configured to receive the composition, allow it to dry, and allow release and penetration of at least a portion of the composition onto and/or into the skin of a user to which it is applied.

In some embodiments, the backing 230 can be a polyurethane backing or other appropriate backing that is configured to provide, e.g., desired structure, strength, or geometry to the article. In another example, the polyurethane carrier can provide the desired structure, moisture vapor transmission properties and porosity. In some embodiments, the backing 230 can reduce and/or eliminate the stickiness of an adjacent adhesive layer. For example, the backing 230 can reduce and/or eliminate the stickiness of the adhesive layer 220. In some embodiments, the adhesive layer 220 can be an acrylic adhesive, a rubber adhesive, or other appropriate adhesive. In some embodiments, the backing 230 can increase the stiffness of the adhesive applicator article 200 during fabrication and/or use. In addition to or instead of increasing stiffness, the backing 230 can be transparent. In incidences when the backing 230 is transparent, a user can see the design formed by the composition (e.g., the composition 60 of FIG. 4A). In some embodiments, when the backing 230 is transparent, the backing 230 can allow an increase in the breathability of between the skin of the user and the external environment.

In some embodiments, the adhesive applicator article 200 is configured to provide proper structure for printing, manufacturing, storage, and/or application of a composition comprising a semi-permanent colorant such as those described herein.

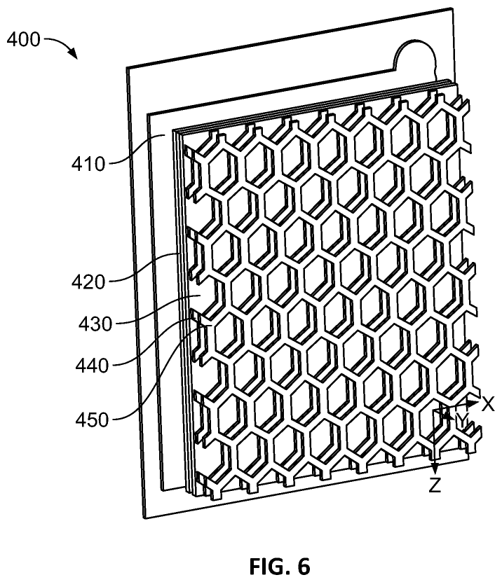

FIG. 6 illustrates an adhesive applicator article according to another embodiment of the present disclosure. As shown in FIG. 6, in some embodiments, the adhesive applicator article 400 comprises a first adhesive layer 420; a first backing 430; a second adhesive layer 440, and a second backing 450. The first adhesive layer 420 can, in some embodiments, be analogous to the adhesive layer 20, the first adhesive layer 120, and/or the adhesive layer 220 described in connection with FIGS. 4A, 4B, 4C, and/or 5 respectively. The liner 410 can, in some embodiments, be analogous to the liner 10, the liner 110, and/or the liner 210 described in connection with FIGS. 4A, 4B, 4C, and/or 5 respectively. The first backing 430 can, in some embodiments, be analogous to the adhesive backing 30, the first backing 130, and/or the backing 230 described in connection with FIGS. 4A, 4B, 4C, and/or 5 respectively. The second adhesive layer 440 can, in some embodiments, be analogous to the adhesive layer 20, the second adhesive layer 140, and/or the adhesive layer 220 described in connection with FIGS. 4A, 4B, 4C, and/or 5 respectively. The second backing 450 can, in some embodiments, be analogous to the adhesive backing 30, the second backing 150, and/or the backing 230 described in connection with FIGS. 4A, 4B, 4C, and/or 5 respectively.

In some embodiments, the second backing is 450 strongly adhered to the second adhesive layer 440 such that its removal during application is prevented. In some embodiments, the second backing 450 can provide structure that prevents wrinkling of the adhesive layer (e.g., the first adhesive layer 420 and/or the second adhesive layer 440) during the application period. In some embodiments, the second adhesive layer 440 is a portion of the first backing 430. In some embodiments, the second backing 450 is a portion of the first backing 430. In some embodiments, the second adhesive layer 440 and the second backing 450 are a portion of the first backing 430. In some embodiments, a portion of a surface (e.g., the first portion 140a and/or the second portion 140b in connection with FIG. 4B) can include a perimeter, a portion of a perimeter, a grid, a section, and the like. In some embodiments, a portion is a honeycomb pattern, for example, as shown in FIG. 6.

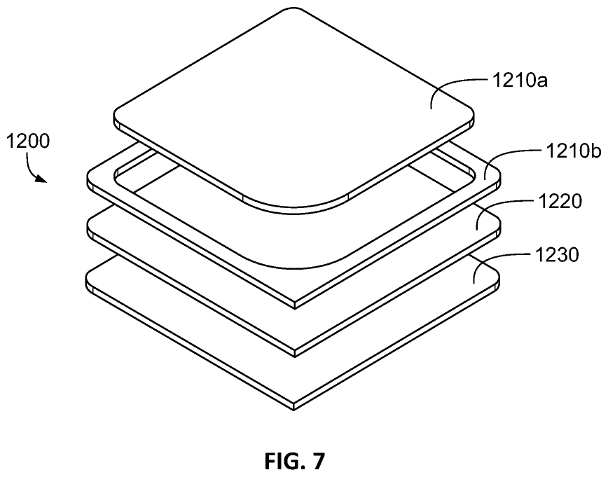

FIG. 7 is an exploded perspective view of an adhesive applicator article 1200 according to another embodiment of the present disclosure. FIG. 7 illustrates a first liner 1210a, a second liner 1210b, an adhesive layer 1220, and a backing 1230. The first liner 1210a and/or the second liner 1210b can, in some embodiments, be analogous to the liner 10, the liner 110, the liner 210, and/or the liner 410 described in connection with 4A, 4B, 4C, 5, and 6 respectively. The adhesive layer 1220 can, in some embodiments, be analogous to the adhesive layer 20, the first adhesive layer 120, the second adhesive layer 140, the adhesive layer 220, the first adhesive layer 420, and/or the second adhesive layer 440 described in connection with 1, 2A, 2B, 3, and 4 respectively. The backing 1230 can, in some embodiments, be analogous to the adhesive backing 30, the first backing 130, the backing 230, and/or the first backing 430, described in connection with 4A, 4B, 4C, 5, and 6 respectively.

In some embodiments, the first liner 1210a can be removable. For example, the first liner 1210a can be removed by a user during the application process. For instance, a user can position the adhesive applicator article 1200 and remove the first liner 1210a to expose a composition (e.g., the composition 60 of FIG. 4A). In some embodiments, the second liner 1210b can be made from a material that can provide some rigidity. For example, the second liner 1210b can be a liner made from a material that can provide a structural border for the adhesive applicator article 1200. For instance, the composition can be a part of an adhesive layer that can be surrounded by a structural border in the form of the second liner 1210b. The second liner 1210b can be configured to provide proper structure to eliminate or reduce the risk of the adhesive including the composition folding in on itself, e.g., during application, and/or to improve the ease of application of the applicator article to a surface, e.g., skin surface, by a user.

The backing 1230, in some embodiments, can be a polyurethane (PU) backing or other appropriate backing that is configured to provide, e.g., desired structure, strength, or geometry to the adhesive applicator article 1200. In some embodiments, a PU backing 1230 can include a thickness of 1 mil. In other embodiments, a PU backing 1230 can include a thickness of 2.4 mil. However, embodiments are not so limited and the backing 1230 can be more or less than 1 mil or 1.4 mil. In some embodiments, the backing 1230 can reduce and/or eliminate the stickiness of an adjacent adhesive layer. For example, the backing 1230 can reduce and/or eliminate the stickiness of the adhesive layer 1220. In some embodiments, the backing 1230 can increase the stiffness of the adhesive applicator article 1200 during fabrication and/or use. An increased stiffness can reduce the risk of damage to the design formed by the composition. In some example embodiments, the backing 1230 can be comprised of a transparent material.

FIG. 8 is another exploded perspective view of an adhesive applicator article 1300 according to another embodiment of the present disclosure. The adhesive applicator article 1300 can be an occlusion patch article and referred to herein as occlusion patch article 1300. FIG. 8 illustrates an occlusion patch article 1300. An occlusion patch can be used with another adhesive applicator article such as the adhesive applicator article 1200 described in connection with FIG. 7. For example, the adhesive applicator article 1300 can be positioned over the adhesive applicator article 1200 to contain moisture and heat during the application process. For example, an occlusion patch can include dimensions greater than adhesive applicator article 1200 such that it can adhere to the skin arounllimd the perimeter of adhesive applicator article 1200. As shown in FIG. 8, in some embodiments the occlusion patch article 1300 comprises a liner 1310, an adhesive layer 1320; a backing 1330; and a structural layer 1340. The liner 1310 can, in some respects, be analogous to the liner 10, the liner 110, the liner 210, the liner 410, the first liner 1210a and/or the second liner 1210b described in connection with FIGS. 4A, 4B, 4C, 5, 6, and 7 respectively. The adhesive layer 1320 can, in some embodiments, be analogous to the adhesive layer 20, the first adhesive layer 120, the second adhesive layer 140, the adhesive layer 220, the first adhesive layer 420, the second adhesive layer 440, and/or the adhesive layer 1220 described in connection with FIGS. 4A, 4B, 4C, 5, 6, and 7 respectively. The backing 1330 can, in some embodiments, be analogous to the adhesive backing 30, the first backing 130, the second backing 150, the backing 230, the first backing 430, and/or the backing 1230 described in connection with FIGS. 4A, 4B, 4C, 5, 6, and 7 respectively.

In some embodiments, the liner 1310 can be removable. For example, the liner 1310 can be removed by a user during the application process. For instance, a user can position the adhesive applicator article 1300 and remove the liner 1310 to expose a composition (e.g., the composition 60 of FIG. 4A). In another example, a user can position the adhesive applicator article 1200 after removing the liner the first liner 1210a and proceed to position the occlusion patch article 1300 on top of the adhesive applicator article 1200.

In some embodiments, the adhesive layer 1320 can be a double-sided adhesive layer 1320. For example, the adhesive layer 1320 can have a first surface 1321 and a second surface 1322. The first surface 1321 can be abut or otherwise be coupled to the liner 1320 and the second surface 1322 can be opposite the first surface 1321 and abut or otherwise contact the backing 1330. In instances when the adhesive layer 1320 is double-sided, an adhesive can be disposed on both the first surface 1321 and the second surface 1322.

In some embodiments, the backing 1330 is made from a transparent material. In incidences when the backing 1330 is transparent, a user can see the design formed by the composition (e.g., the composition 60 of FIG. 4A). In some embodiments, when the backing 1330 is transparent, the backing 1330 can allow an increase in the breathability of between the skin of the user and the external environment.

The structural layer 1340 can be a portion of the occlusion patch article 1300 that can provide a flexible frame to the composition design. The structural layer 1340 can be a support to protect the composition design and provide structure to the occlusion patch article 1300. In some embodiments the liner 1310 can be removable and/or may be a craft paper coated with silicone on the first surface 1321. In some embodiments, the double-sided adhesive layer 1320 may be a rubber synthetic adhesive such that first surface 1321 adheres to removable liner 1310 and the second surface 322 adheres to the transparent backing 1330. In some embodiments where the backing 1330 is transparent, the backing 1330 may be a polyurethane of thickness mil to 3.0 mil. In some embodiments, layer 340 may be a roll label frame adhering to the periphery of transparent layer 330, of thickness 2-5 mil.

In some embodiments, the occlusion patch article 1300 will have larger dimensions compared to other embodiments discussed herein. For example, the occlusion patch article 1300 can have dimensions greater than the adhesive applicator article 100 described in connection with FIGS. 4B and 4C. For instance, the occlusion patch article 1300 can include dimensions greater than adhesive applicator article 100 such that it can adhere to the skin around the perimeter of adhesive applicator article 100. In some embodiments, when the applicator article 1300 adheres to the skin, the removable liner 1310 will be removed exposing adhesive layer 1320 first surface 1321 such that the first surface 1321 can adhere to the skin. The occlusion patch article 1300 can surround the adhesive layer and/or a layer comprising the composition.

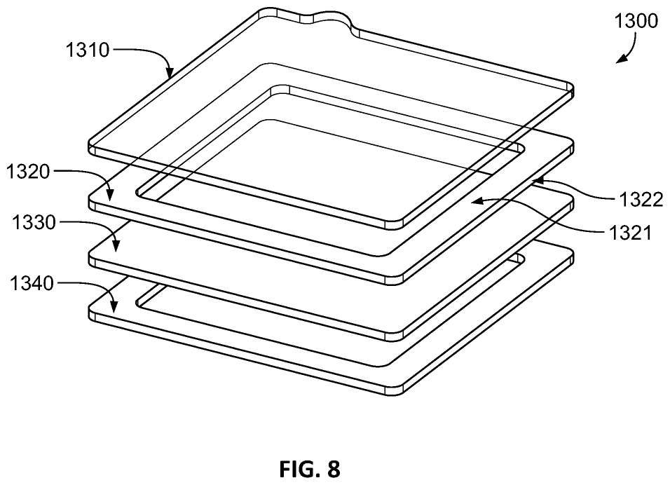

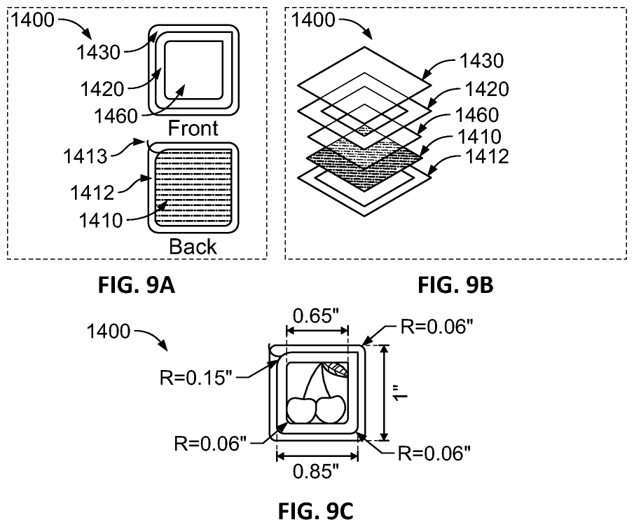

FIG. 9A, FIG. 9B, and FIG. 9C are perspective drawings of an adhesive applicator article 1400 according to an embodiment of the present disclosure. FIG. 9A is a top view of a front and a back of the adhesive applicator article 1400, FIG. 9B is an exploded view of the adhesive applicator article 1400, and FIG. 9C is an example of dimensions of the adhesive applicator article 1400. The example adhesive applicator 1400 can be an example of an occlusion patch article (e.g., the occlusion patch article 1300 described in connection with FIG. 8) with an embedded stack up 1460.

FIG. 9A illustrates a front and a back top view of the adhesive applicator article 1400. The adhesive applicator article 1400 includes a backing 1430. In some embodiments, the backing 1430 is a polyurethane (PU) backing. For instance, the backing 1430 can be a 1 mil PU backing. The adhesive applicator article 1400 can include an adhesive layer 1420. In some embodiments the adhesive layer 1420 is a double sided adhesive that can adhere to the backing 1430 and a stack up 1460. The stack up 1460 can include an adhesive layer, a double sided adhesive layer, and a 1 mil PU backing. The stack up can be positioned on top of a liner 1410. In some embodiments, the liner 1410 is a fluorosilicone (FS) liner. The liner 1410 and/or the border 1412 can be removed by a user to expose the stack up 1460 to the skin of the user. The liner can be coupled to and/or otherwise positioned with a border 1412. The border 1412 can provide a guide for the user as well as structure to the adhesive applicator article 1400. The adhesive applicator article 1400 can further include a pull-tab 1430 to facilitate the removal of the border 1412. For example, the pull-tab 1413 can facilitate the removal of the border 1410. After the removal of the border 1410 using the pull-tab 1413, the user can remove the liner 1410. The liner 1410 can facilitate the preservation and/or the shelf life of the composition.

FIG. 9B illustrates an exploded view of the adhesive applicator article 1400. The exploded view illustrates an example of the different elements of the adhesive applicator article 1400. In this instance, the adhesive applicator article 1400 includes a backing (e.g., a 1 mil PU backing) to couple to an adhesive layer 1420 (e.g., a double-sided adhesive layer 1420), to couple to a stack up 1460. The stack up 1460 can be coupled to a liner 1410 (e.g., a FS liner) which can be removed to expose the stack up 1460 to the skin of a user. The border 1412 can provide structure to the adhesive applicator article 1400 and facilitate the position and adherence to the skin of a user and/or the handling and position to the skin of the user.

FIG. 9C includes an example design with example measurements. While specific numbers are illustrated in FIG. 9, embodiments are not so limited. Multiple different dimensions have been considered and may be more or less than the dimensions illustrated in FIG. 9. As mentioned herein, an occlusion patch article can, in some embodiments, have larger dimensions compared to other embodiments discussed herein. For example, an occlusion patch article (e.g., the adhesive applicator article 1400) can have dimensions greater than the adhesive applicator article 1200 described in connection with FIG. 7.

The adhesive applicator article 1400 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 9C illustrates border (e.g., the border 1412 of FIGS. 9A and 9B) with a dimension of 1.0 Inch (in), an adhesive layer (e.g., the adhesive layer 1420 of FIGS. 9A and 9B) including a dimension of 0.85 in, and a stack up (e.g., the stack up 1460 of FIGS. 9A and 9B) including a dimension of 0.65 in. The adhesive layer and/or the stack up can include a design comprising a composition (e.g., the composition 60 as described above in connection with FIG. 4A). The border can have a radius of curvature (R) R=0.15 in, adhesive layer can have a radius of curvature of R=0.06 in, and the stack up can have a radius of curvature of R=0.06 in.

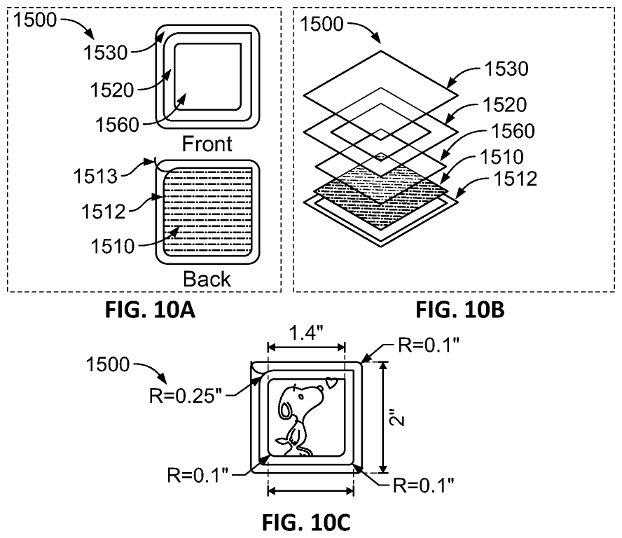

FIG. 10A, FIG. 10B, and FIG. 10C are perspective drawings of an adhesive applicator article 1500 according to an embodiment of the present disclosure. FIG. is a top view of a front and a back of the adhesive applicator article 1500, FIG. is an exploded view of the adhesive applicator article 1500, and FIG. 10C is an example of dimensions of the adhesive applicator article 1500. The example adhesive applicator 1500 can be an example of an occlusion patch article (e.g., the occlusion patch article 1300 described in connection with FIG. 8) with an embedded stack up 1560.

FIG. 10A illustrates a front and a back top view of the adhesive applicator article 1500. The adhesive applicator article 1500 includes a backing 1530. In some embodiments, the backing 1530 is a PU backing. For instance, the backing 1530 can be a 1 mil PU backing. The adhesive applicator article 1500 can include an adhesive layer 1520. In some embodiments the adhesive layer 1520 is a double sided adhesive that can adhere to the backing 1530 and a stack up 1560. The stack up 1560 can include an adhesive layer, a double sided adhesive layer, and a 1 mil PU backing. The stack up can be positioned on top of a liner 1510. In some embodiments, the liner 1510 is a FS liner. The liner 1510 and/or the border 1512 can be removed by a user to expose the stack up 1560 to the skin of the user. The liner can be coupled to and/or otherwise positioned with a border 1512. The border 1512 can provide a guide for the user as well as structure to the adhesive applicator article 1500. The adhesive applicator article 1500 can further include a pull-tab 1530 to facilitate the removal of the border 1512. For example, the pull-tab 1513 can facilitate the removal of the border 1510. After the removal of the border 1510 using the pull-tab 1513, the user can remove the liner 1510. The liner 1510 can facilitate the preservation and/or the shelf life of the composition.

FIG. 10B illustrates an exploded view of the adhesive applicator article 1500. The exploded view illustrates an example of the different elements of the adhesive applicator article 1500. In this instance, the adhesive applicator article 1500 includes a backing (e.g., a 1 mil PU backing) to couple to an adhesive layer 1520 (e.g., a double-sided adhesive layer 1520), to couple to a stack up 1560. The stack up 1560 can be coupled to a liner 1510 (e.g., a FS liner) which can be removed to expose the stack up 1560 to the skin of a user. The border 1512 can provide structure to the adhesive applicator article 1500 and facilitate the position and adherence to the skin of a user and/or the handling and position to the skin of the user.

FIG. 10C includes an example design with example measurements. While specific numbers are illustrated in FIG. 10, embodiments are not so limited. Multiple different dimensions have been considered and may be more or less than the dimensions illustrated in FIG. 10. As mentioned herein, an occlusion patch article can, in some embodiments, have larger dimensions compared to other embodiments discussed herein. For example, an occlusion patch article (e.g., the adhesive applicator article 1500) can have dimensions greater than the adhesive applicator article 1200 described in connection with FIG. 7.

The adhesive applicator article 1500 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

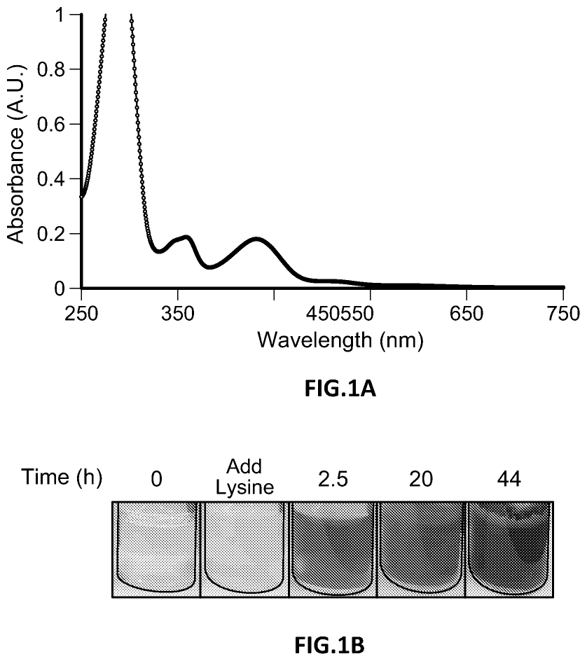

The embodiment illustrated in FIG. 10C illustrates border (e.g., the border 1512 of FIGS. 10A and 10B) with a dimension of 2.0 Inches (in), an adhesive layer (e.g., the adhesive layer 1520 of FIGS. 10A and 10B) including a dimension of 1.7 in, and a stack up (e.g., the stack up 1560 of FIGS. 10A and 1B) including a dimension of 1.4 in. The adhesive layer and/or the stack up can include a design comprising a composition (e.g., the composition 60 as described above in connection with FIG. 4A). The border can have a radius of curvature (R) R=0.25 in, adhesive layer can have a radius of curvature of R=0.1 in, and the stack up can have a radius of curvature of R=0.1 in.

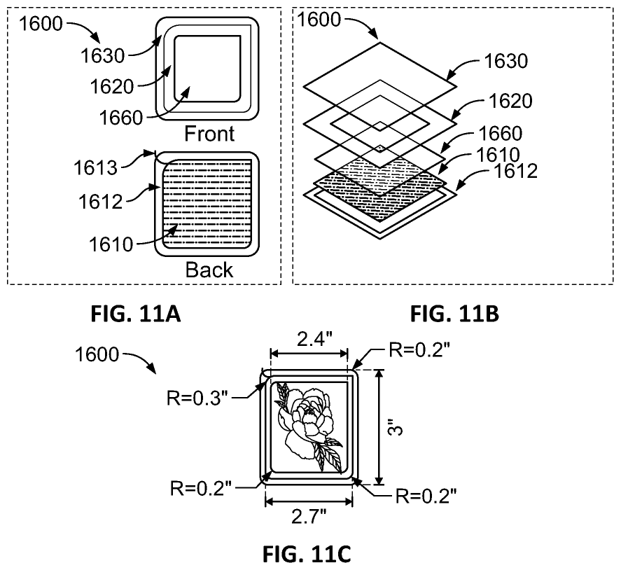

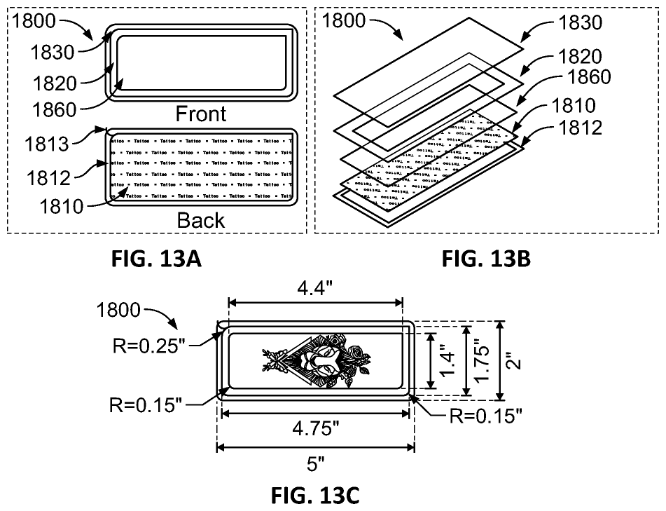

FIG. 11A, FIG. 11B, and FIG. 11C are perspective drawings of an adhesive applicator article 1600 according to an embodiment of the present disclosure. FIG. 11A is a top view of a front and a back of the adhesive applicator article 1600, FIG. 11B is an exploded view of the adhesive applicator article 1600, and FIG. 11C is an example of dimensions of the adhesive applicator article 1600. The example adhesive applicator 1600 can be an example of an occlusion patch article (e.g., the occlusion patch article 1300 described in connection with FIG. 13) with an embedded stack up 1660.

FIG. 11A illustrates a front and a back top view of the adhesive applicator article 1600. The adhesive applicator article 1600 includes a backing 1630. In some embodiments, the backing 1630 is a PU backing. For instance, the backing 1630 can be a 1 mil PU backing. The adhesive applicator article 1600 can include an adhesive layer 1620. In some embodiments the adhesive layer 1620 is a double sided adhesive that can adhere to the backing 1630 and a stack up 1660. The stack up 1660 can include an adhesive layer, a double sided adhesive layer, and a 1 mil PU backing. The stack up can be positioned on top of a liner 1610. In some embodiments, the liner 1610 is a FS liner. The liner 1610 and/or the border 1612 can be removed by a user to expose the stack up 1660 to the skin of the user. The liner can be coupled to and/or otherwise positioned with a border 1612. The border 1612 can provide a guide for the user as well as structure to the adhesive applicator article 1600. The adhesive applicator article 1600 can further include a pull-tab 1630 to facilitate the removal of the border 1612. For example, the pull-tab 1613 can facilitate the removal of the border 1610. After the removal of the border 1610 using the pull-tab 1613, the user can remove the liner 1610. The liner 1610 can facilitate the preservation and/or the shelf life of the composition.

FIG. 11B illustrates an exploded view of the adhesive applicator article 1600. The exploded view illustrates an example of the different elements of the adhesive applicator article 1600. In this instance, the adhesive applicator article 1600 includes a backing (e.g., a 1 mil PU backing) to couple to an adhesive layer 1620 (e.g., a double-sided adhesive layer 1620), to couple to a stack up 1660. The stack up 1660 can be coupled to a liner 1610 (e.g., a FS liner) which can be removed to expose the stack up 1660 to the skin of a user. The border 1612 can provide structure to the adhesive applicator article 1600 and facilitate the position and adherence to the skin of a user and/or the handling and position to the skin of the user.

FIG. 11C includes an example design with example measurements. While specific numbers are illustrated in FIG. 11, embodiments are not so limited. Multiple different dimensions have been considered and may be more or less than the dimensions illustrated in FIG. 11. As mentioned herein, an occlusion patch article can, in some embodiments, have larger dimensions compared to other embodiments discussed herein. For example, an occlusion patch article (e.g., the adhesive applicator article 1600) can have dimensions greater than the adhesive applicator article 1200 described in connection with FIG. 7.

The adhesive applicator article 1500 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

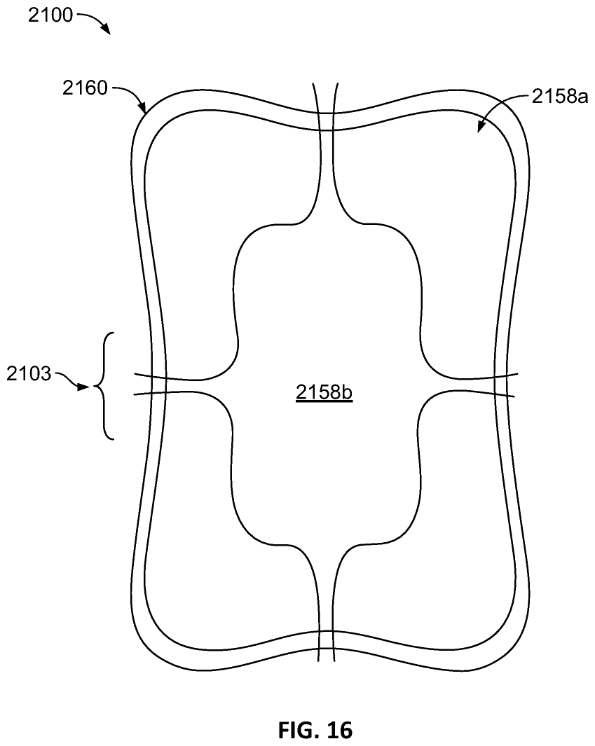

The embodiment illustrated in FIG. 11C illustrates border (e.g., the border 1612 of FIGS. 16A and 16B) with a dimension of 3.0 Inches (in), an adhesive layer (e.g., the adhesive layer 1620 of FIGS. 16A and 16B) including a dimension of 2.7 in, and a stack up (e.g., the stack up 1660 of FIGS. 16A and 16B) including a dimension of 2.4 in. The adhesive layer and/or the stack up can include a design comprising a composition (e.g., the composition 60 as described above in connection with FIG. 4A). The border can have a radius of curvature (R) R=0.3 in, adhesive layer can have a radius of curvature of R=0.2 in, and the stack up can have a radius of curvature of R=0.2 in.

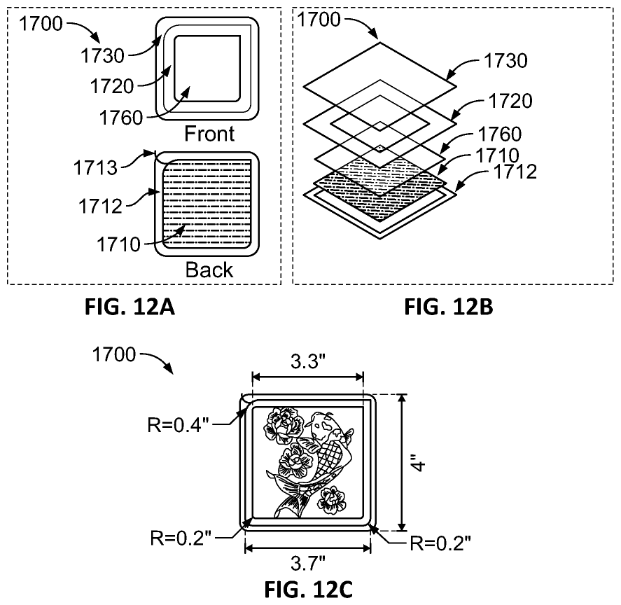

FIG. 12A, FIG. 12B, and FIG. 12C are perspective drawings of an adhesive applicator article 1700 according to an embodiment of the present disclosure. FIG. 12A is a top view of a front and a back of the adhesive applicator article 1700, FIG. 12B is an exploded view of the adhesive applicator article 1700, and FIG. 12C is an example of dimensions of the adhesive applicator article 1700. The example adhesive applicator 1700 can be an example of an occlusion patch article (e.g., the occlusion patch article 1300 described in connection with FIG. 13) with an embedded stack up 1760.

FIG. 12A illustrates a front and a back top view of the adhesive applicator article 1700. The adhesive applicator article 1700 includes a backing 1730. In some embodiments, the backing 1730 is a PU backing. For instance, the backing 1730 can be a 1 mil PU backing. The adhesive applicator article 1700 can include an adhesive layer 1720. In some embodiments the adhesive layer 1720 is a double sided adhesive that can adhere to the backing 1730 and a stack up 1760. The stack up 1760 can include an adhesive layer, a double sided adhesive layer, and a 1 mil PU backing. The stack up can be positioned on top of a liner 1710. In some embodiments, the liner 1710 is a FS liner. The liner 1710 and/or the border 1712 can be removed by a user to expose the stack up 1760 to the skin of the user. The liner can be coupled to and/or otherwise positioned with a border 1712. The border 1712 can provide a guide for the user as well as structure to the adhesive applicator article 1700. The adhesive applicator article 1700 can further include a pull-tab 1730 to facilitate the removal of the border 1712. For example, the pull-tab 1713 can facilitate the removal of the border 1710. After the removal of the border 1710 using the pull-tab 1713, the user can remove the liner 1710. The liner 1710 can facilitate the preservation and/or the shelf life of the composition.

FIG. 12B illustrates an exploded view of the adhesive applicator article 1700. The exploded view illustrates an example of the different elements of the adhesive applicator article 1700. In this instance, the adhesive applicator article 1700 includes a backing (e.g., a 1 mil PU backing) to couple to an adhesive layer 1720 (e.g., a double-sided adhesive layer 1720), to couple to a stack up 1760. The stack up 1760 can be coupled to a liner 1710 (e.g., a FS liner) which can be removed to expose the stack up 1760 to the skin of a user. The border 1712 can provide structure to the adhesive applicator article 1700 and facilitate the position and adherence to the skin of a user and/or the handling and position to the skin of the user.

FIG. 12C includes an example design with example measurements. While specific numbers are illustrated in FIG. 12, embodiments are not so limited. Multiple different dimensions have been considered and may be more or less than the dimensions illustrated in FIG. 12. As mentioned herein, an occlusion patch article can, in some embodiments, have larger dimensions compared to other embodiments discussed herein. For example, an occlusion patch article (e.g., the adhesive applicator article 1700) can have dimensions greater than the adhesive applicator article 1200 described in connection with FIG. 12.

The adhesive applicator article 1700 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 12C illustrates border (e.g., the border 1712 of FIGS. 12A and 12B) with a dimension of 4.0 Inches (in), an adhesive layer (e.g., the adhesive layer 1720 of FIGS. 12A and 12B) including a dimension of 3.7 in, and a stack up (e.g., the stack up 1760 of FIGS. 12A and 12B) including a dimension of 3.3 in. The adhesive layer and/or the stack up can include a design comprising a composition (e.g., the composition 60 as described above in connection with FIG. 4A). The border can have a radius of curvature (R) R=0.4 in, adhesive layer can have a radius of curvature of R=0.2 in, and the stack up can have a radius of curvature of R=0.2 in.

FIG. 13A, FIG. 13B, and FIG. 13C are perspective drawings of an adhesive applicator article 1800 according to an embodiment of the present disclosure. FIG. 13A is a top view of a front and a back of the adhesive applicator article 1800, FIG. 13B is an exploded view of the adhesive applicator article 1800, and FIG. 13C is an example of dimensions of the adhesive applicator article 1800. The example adhesive applicator 1800 can be an example of an occlusion patch article (e.g., the occlusion patch article 1300 described in connection with FIG. 8) with an embedded stack up 1860.

FIG. 13A illustrates a front and a back top view of the adhesive applicator article 1800. The adhesive applicator article 1800 includes a backing 1830. In some embodiments, the backing 1830 is a PU backing. For instance, the backing 1830 can be a 1 mil PU backing. The adhesive applicator article 1800 can include an adhesive layer 1820. In some embodiments the adhesive layer 1820 is a double sided adhesive that can adhere to the backing 1830 and a stack up 1860. The stack up 1860 can include an adhesive layer, a double sided adhesive layer, and a 1 mil PU backing. The stack up can be positioned on top of a liner 1810. In some embodiments, the liner 1810 is a FS liner. The liner 1810 and/or the border 1812 can be removed by a user to expose the stack up 1860 to the skin of the user. The liner can be coupled to and/or otherwise positioned with a border 1812. The border 1812 can provide a guide for the user as well as structure to the adhesive applicator article 1800. The adhesive applicator article 1800 can further include a pull-tab 1830 to facilitate the removal of the liner 1820. For example, the pull-tab 1813 can facilitate the removal of the border 1810. After the removal of the border 1810 using the pull-tab 1813, the user can remove the liner 1810. The liner 1810 can facilitate the preservation and/or the shelf life of the composition.

FIG. 13B illustrates an exploded view of the adhesive applicator article 1800. The exploded view illustrates an example of the different elements of the adhesive applicator article 1800. In this instance, the adhesive applicator article 1800 includes a backing (e.g., a 1 mil PU backing) to couple to an adhesive layer 1820 (e.g., a double-sided adhesive layer 1820), to couple to a stack up 1860. The stack up 1860 can be coupled to a liner 1810 (e.g., a FS liner) which can be removed to expose the stack up 1860 to the skin of a user. The border 1812 can provide structure to the adhesive applicator article 1800 and facilitate the position and adherence to the skin of a user and/or the handling and position to the skin of the user.

FIG. 13C includes an example design with example measurements. While specific numbers are illustrated in FIG. 13, embodiments are not so limited. Multiple different dimensions have been considered and may be more or less than the dimensions illustrated in FIG. 13. As mentioned herein, an occlusion patch article can, in some embodiments, have larger dimensions compared to other embodiments discussed herein. For example, an occlusion patch article (e.g., the adhesive applicator article 1800) can have dimensions greater than the adhesive applicator article 1200 described in connection with FIG. 7.

The adhesive applicator article 1800 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 13C illustrates border (e.g., the border 1812 of FIGS. 13A and 13B) with a dimension of length 5 Inches (in) and width 2.0 in, an adhesive layer (e.g., the adhesive layer 1820 of FIGS. 13A and 183) including a dimension of length 4.75 in and width 1.75 in, and a stack up (e.g., the stack up 1860 of FIGS. 13A and 13B) including a dimension of length 4.4 in and a width of 1.4 in. The adhesive layer and/or the stack up can include a design comprising a composition (e.g., the composition 60 as described above in connection with FIG. 4A). The border can have a radius of curvature (R) R=0.25 in, adhesive layer can have a radius of curvature of R=0.15 in, and the stack up can have a radius of curvature of R=0.15 in.

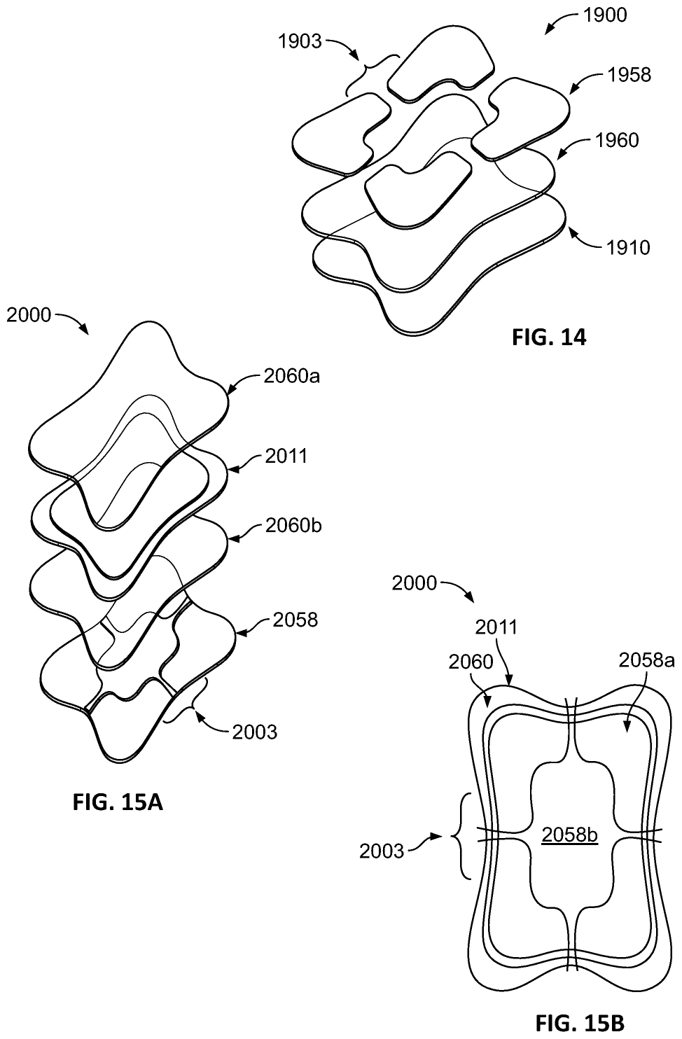

FIG. 14 is another example of an adhesive applicator article 1900 according to another embodiment of the present disclosure. The adhesive applicator article 1900 can include an aperture 1903, one or more portions of a backing 1958, a stack up 1960, and a liner 1910. While one aperture 1903 is identified in FIG. 14, there can be more or less than one aperture 1903. The stack up 1960 can be analogous to the stack up 1460, 1560, 1660, 1760, and 1860 described in connection with FIGS. 9, 10, 11, 12, and 13 respectively. The liner can be an FS liner which can be selectively removable by a user to expose the stack up 1960 to the skin of the user. The backing 1958 can be a removable backing. In some embodiments, the backing 1958 can be a removable paper backing that can be removed by a user during the application of the adhesive applicator article 1900. FIG. 14 labels a portion of the backing 1958, however, as illustrated in FIG. 14, there are four portions of the backing 1958. While four portions of the backing 1958 are illustrated in the example of the adhesive applicator article 1900, embodiments are not so limited and there may be more or less than four portions of backing 1958.

The adhesive applicator article 1900 can include one or more apertures 1903 which can facilitate the stiffness of the adhesive applicator article 1900 during the application of the adhesive applicator article 1900 onto a body part of a user. For example, the adhesive applicator article 1900 can be a design for large designs (e.g., oversized tattoo designs). As illustrated in FIG. 14 the shape of the adhesive applicator article 1900 can be curved to provide a butterfly shape. The butterfly shape of the adhesive applicator article 1900 can adapt to the contours of the body. The butterfly shape in combination with the aperture(s) 1903 can prevent and/or reduce lifting and delamination of the adhesive applicator article 1900 once an oversized tattoo applicator is applied on any convex body surfaces (e.g., biceps, forearms, thighs, shoulder, etc.). The incorporation of a structural layer such as the paper backing 1958 on top of the adhesive applicator article 1900 can provide further structural support to prevent wrinkles and/or facilitate further wrinkle free application. A user can remove the FS liner 1910 to expose the adhesive layer included in (e.g., in contact with) the stack up 1960 to apply the design to the skin. The user may smooth out any wrinkles by applying pressure to the paper backing 1958 and/or by smoothing out the paper backing 1958. When the user is satisfied with the application, they can remove the one or more superficial paper backing 1958 portions.

In another example embodiment, structural support can be further provided to the adhesive applicator article 1900 by cutting through a FS liner on the adhesive side of the stack up and/or providing a border. These embodiments are discussed in connection with FIGS. 15A, 16B, and 16.

FIGS. 15A and 15B illustrate perspective views of an example adhesive applicator article 2000 according to another embodiment of the present disclosure. FIG. 15A is an exploded view of the layers of the example adhesive applicator article 2000 described in connection with FIG. 15B. FIG. 15B is a bottom view of the example adhesive applicator article 2000.

FIG. 15A shows an exploded view of layers of the adhesive applicator article 2000. The adhesive applicator article 2000 includes one or more apertures 2003, a liner 2060a (e.g., a 1 mil PU liner), an adhesive layer (e.g., a double sided adhesive layer) 2011, a stack up 2060b, and an FS liner 2058.

While one aperture 2003 is identified in FIGS. 15A and 15B, there can be more or less than one aperture 2003. The stack up 2060 of FIGS. 15A and 15B can be analogous to the stack up 1460, 1560, 1660, 1760, and 1860 described in connection with FIGS. 9, 10, 11, 12, and 13 respectively. The adhesive layer 2011 of FIGS. 15A and 15B can provide structure and rigidity to the adhesive applicator article 2000.

The FS liner 2058 can be removed in portions as described in connection with FIG. 15A and the adhesive layer 2011 can provide support to the adhesive applicator article 2000 during application and/or during the removal of the FS liner 2058. FIGS. 15A and 15B label a portion of the FS liner 2058, however, as illustrated in FIGS. 15A and 15B, there are four portions of the backing 2058. While four portions of the backing 2058 are illustrated in the example of the adhesive applicator article 2000, embodiments are not so limited and there may be more or less than four portions of backing 2058.

FIG. 15B illustrates layer 2060 which is an overlapped view of liner 2060a combined with stack up 2060b described in connection with FIG. 15A. FIG. 15B further illustrates the adhesive layer 2011 which can provide support during application. FIG. 15B further illustrates FS liner 2058 in two sections 2058a and 2058b. A user can remove the center portion 2058b to expose the design imprinted on the stack up 2060. The user can position the design onto the skin and smooth out wrinkles. When the user is satisfied with the placement, the user can remove the four portions of the FS liner 2058a. During this application process, the double sided adhesive layer 2011 can provide a strong structural border. The example adhesive applicator article 2000 can include extending one or more of the apertures 2003 through the FS liner 2058 such that the FS liner 2058 can act as a structural border. The butterfly shape of the adhesive applicator article 2000 can adapt to the contours of the body

The butterfly shape in combination with the aperture(s) 2003 extending through the FS liner 2058 can prevent and/or reduce lifting and delamination of the adhesive applicator article 2000 once an oversized tattoo applicator is applied on any convex body surfaces (e.g., biceps, forearms, thighs, shoulder, etc.). The incorporation of a structural layer 2011 can provide further structural support to prevent wrinkles and/or facilitate further wrinkle free application.

FIG. 11 is another example of an adhesive applicator article 2100 according to another embodiment of the present disclosure. FIG. 11 is a bottom up view that is similar to FIGS. 15A and 15B except it is without the adhesive layer to provide a structural border. Adhesive applicator article 2100 includes an overlapped combination of a stack up and an FS liner 2160. The adhesive applicator article 2100 can include one or more apertures 2103, one or more portions of a backing 2158a and a center portion of the backing 2058b. While one aperture 2103 is identified in FIG. 11, there can be more or less than one aperture 2103. The stack up included in 2160 can be analogous to the stack up 1460, 1560, 1660, 1760, and 1860 described in connection with FIGS. 9, 10, 11, 12, and 13 respectively.

The backing 2158 can be a removable backing. In some embodiments, the backing 2158 can be a removable paper backing and/or and FS liner that can be removed by a user during the application of the adhesive applicator article 2100. FIG. 11 labels a portion of the backing 2158, however, as illustrated in FIG. 11, there are four portions of the backing 2158. While four portions of the backing 2158 are illustrated in the example of the adhesive applicator article 2100, embodiments are not so limited and there may be more or less than four portions of backing 2158. The example adhesive applicator article 2100 can include extending one or more of the apertures 2103 through the FS liner (not illustrated) on the adhesive side of the stack up 2160 in order to provide structural support to the adhesive applicator article 2100.

The extension of the one or more of the apertures 2103 through the FS liner on the adhesive side of the stack up included in 2160 can provide support and better adhesion by removing the backing 2058a and 2158b into smaller parts of the article 2100 from lifting off of the users skin when applied to convex surfaces. The butterfly shape of the adhesive applicator article 2100 can adapt to the contours of the body. The butterfly shape in combination with the aperture(s) 2103 extending through the FS liner can prevent and/or reduce lifting and delamination of the adhesive applicator article 2100 once an oversized adhesive applicator is applied on any convex body surfaces (e.g., biceps, forearms, thighs, shoulder, etc.). The incorporation of a structural layer such as the paper backing 2158 on top of the adhesive applicator article 2100 can provide further structural support to prevent wrinkles and/or facilitate further wrinkle free application.

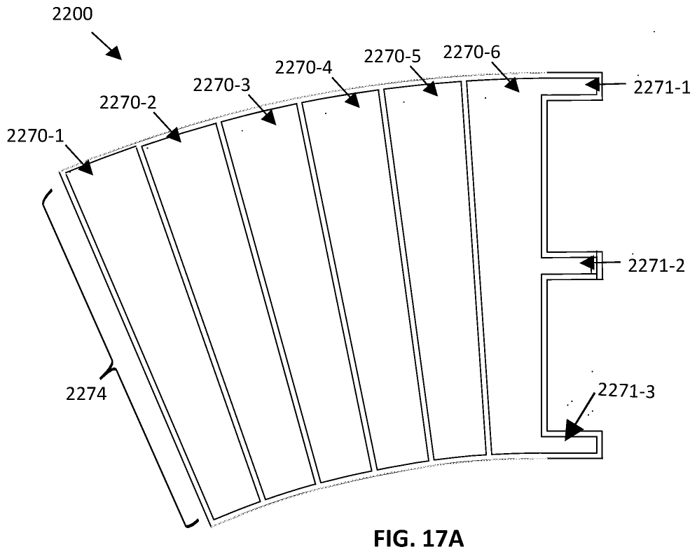

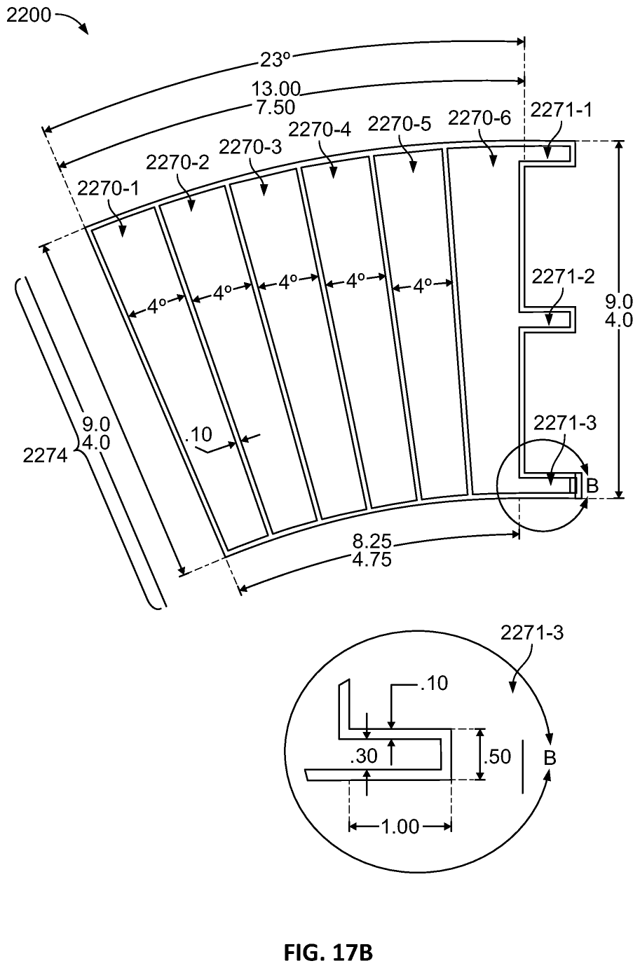

FIGS. 17A and 17B illustrate another example of an adhesive applicator article 2200 according to another embodiment of the present disclosure. The example of adhesive applicator article 2200 is an example of an adhesive applicator designed to provide the placement of a design to a convex portion of a user. For example, the adhesive applicator article 2200 can be applied to such parts of a user's body as a forearm, a thigh, a shoulder, a bicep, etc.

Both FIG. 12A and FIG. 12B include a plurality of panels that can include a design comprising a composition (e.g., the composition 60 described in connection with FIG. 4A). The panels 2270-1, 2270-2, 2270-3, 2270-4, 2270-5, and 2270-6 can be collectively referred to herein as the panels 2270. While six panels 2270 are illustrated in FIG. 12A and FIG. 12B, embodiments are not so limited and embodiments can include more or less than six panels 2270.

Although not specifically labeled as to not obscure embodiments of the disclosure, the adhesive applicator article 2200 can include any of the elements described in connection with FIGS. 4-18. For example, the adhesive applicator article 2200 can include adhesive layers, backings, liners, compositions, and designs, among other elements as described herein. The adhesive applicator article 2200 can include an external layer. The external layer may be comprised of can include adhesive layers, backings, liners, etc. The external layer can be opposite the portion of the adhesive applicator article 2200 that contacts the skin of the user.

As mentioned, the adhesive applicator article 2200 can be used for a convex portion of a user's body such as a forearm. The adhesive applicator article 2200 illustrated by FIGS. 17A and 17B can include one or more tabs 2271-1, 2271-2, and 2271-3 which can be referred to herein as tabs 2271. While three tabs 2271 are illustrated in FIG. 12A and FIG. 12B, embodiments are not so limited and embodiments can include more or less than three tabs 2271. The tabs 2271 are included to prevent and/or reduce delamination and to give the user a direction of application. For example, a user can apply the adhesive applicator article 2200 to their forearm and, using the tabs 2271, wrap the adhesive applicator article 2200 such that the tabs 2271 contact the portion 2274. The portion 2274 can contacted by the tabs 2271 can include the external layer. The tabs 2271 can adhere to the outer layer of the adhesive applicator article 2200 after wrapping around the forearm. The tabs 2271 contacting the portion 2274 can keep the adhesive applicator article 2200 on the skin of the user even during movement of the user.

FIG. 12B illustrates example dimensions (all dimensions are illustrated in inches). For example, the top of the tabs 2270 span from 7.5 in to 13.0 in (not including the tabs 2271) at an arc of 23 degrees. The bottom of the tabs 2270 span 8.25 in including the tabs 2271 and 4.75 in not including the tabs 2271. The height of the adhesive applicator article 2200 is can be (for example) between 9.0 and 4.0 in. Each panel 2270 includes a 4 degree arc with a 0.10 in space that can act as a border. FIG. 4C includes a zoomed in perspective of the tab 2271-3. The tab 2271-3 has the dimension of a 0.10 in border where each tab includes a width of 0.50 in and a length of 1.0 in with a 0.30 in space between the borders of each tab 2271. While specific numbers are listed and illustrated herein, embodiments are not so limited, and different sizes and configurations of adhesive applicator article 2200 have been contemplated to accommodate users of differing sizes.

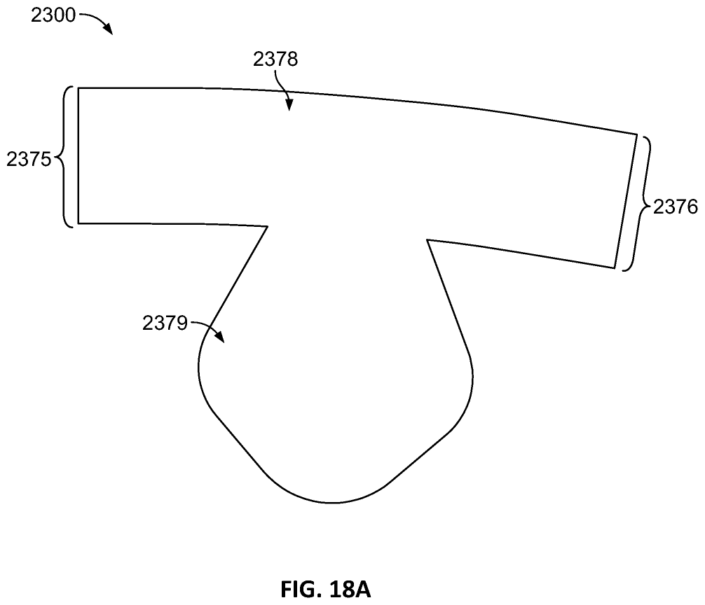

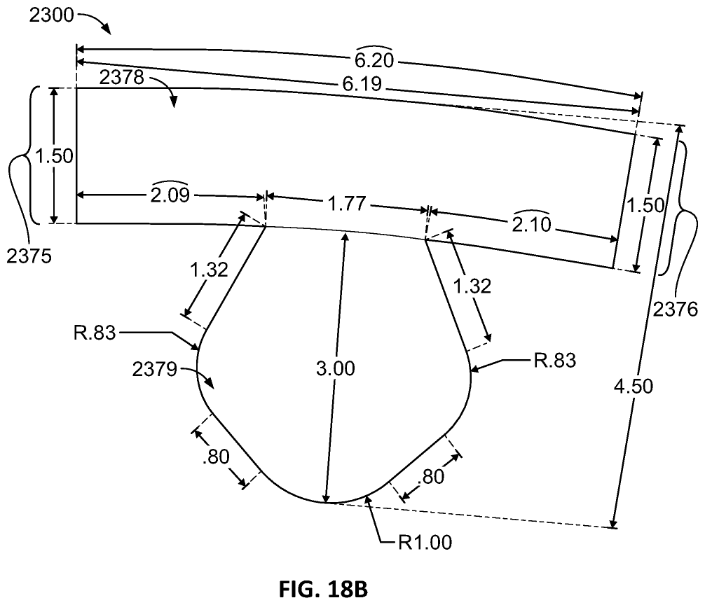

FIGS. 18A and 18B illustrate another example of an adhesive applicator article 2300 according to another embodiment of the present disclosure. The example of adhesive applicator article 2300 is an example of an adhesive applicator designed to provide the placement of a design to a convex and/or moving portion of a user. For example, the adhesive applicator article 2300 can be applied to such parts of a user's hand, foot, wrist and/or ankle, etc.

Although not specifically labeled as to not obscure embodiments of the disclosure, the adhesive applicator article 2300 can include any of the elements described in connection with FIGS. 4-18. For example, the adhesive applicator article 2300 can include adhesive layers, backings, liners, compositions, and designs, among other elements as described herein. The adhesive applicator article 2300 can include an external layer. The external layer may be comprised of can include adhesive layers, backings, liners, etc. The external layer can be opposite the portion of the adhesive applicator article 2300 that contacts the skin of the user.

Both FIGS. 18A and 18B include a first portion 2378 having a left side 2375 (e.g., a first side), a right side 2376 (e.g., a second side), and a second portion 2379. The first portion 2378 and/or the second portion 2379 can include a composition (e.g., the composition 60 described in connection with FIG. 4A). The left side 2375 and the right side 2376 are included to prevent and/or reduce delamination and to give the user a direction of application. For example, a user can apply the adhesive applicator article 2300 to their hand and/or wrist and, using the left side 2375 and the right side 2376, wrap the adhesive applicator article 2300 such that left side 2375 contacts the right side 2376. The left side 2375 and the right side 2376 can adhere to the outer layer of each other after wrapping around the wrist. The left side 2375 and the right side 2376 can keep the adhesive applicator article 2300 on the skin of the user even during movement of the user. Using these methods the second portion 2379 can be positioned to provide a design to the top of a user's hand and the first portion 2378 can be positioned to provide a design to the wrist of a user.

FIG. 13B illustrates example dimensions (all dimensions are illustrated in inches). For example, from the left side 2375 to the right side 2376 the first portion 2378 can be 6.19 in and 6.20 in including the curved portion. The width of the left side 2375 and the right side 2376 can be 1.50 in and the distance from the left side 2375 and the right side 2376 to the second portion 2376 can be 2.10 in. The flat portion of the second portion 2379 can be 1.32 in while the curved portions have an R=0.83 in. The end of the second portion 2379 can include an R=1.00 in with flat portions of 0.80 in between. The distance from the top of the first portion 2378 to the bottom of the second portion 2379 can be 4.50 in. While specific numbers are listed and illustrated herein, embodiments are not so limited, and different sizes and configurations of adhesive applicator article 2300 have been contemplated to accommodate users of differing sizes.

In some of any of the above embodiments, two or more compositions can be applied to an applicator, e.g., any of the applicators described herein. For example, a first composition and a second composition can be applied to an applicator; a first composition, a second composition, and a third composition can be applied to an applicator; or a first composition, a second composition, a third composition, and a fourth composition can be applied to an applicator. In some embodiments, each composition has a different semi-permanent colorant. In some embodiments each semi-permanent colorant has a different color.

Also provided herein are methods for applying a temporary tattoo ink to an article.

In some embodiments, the composition (e.g., Composition A as described herein or the composition 60 of FIG. 4A) is applied to the first surface 121 of the first adhesive layer 120 as shown in FIG. 4B. In some embodiments two or more composi

In some embodiments, the composition 160 is applied by inkjet printing.

In some embodiments, the composition (e.g., the composition 60 of FIG. 4A) is applied to the first surface of the first adhesive layer 220 described in connection with FIG. 5.

In some embodiments, the composition 260 is applied by inkjet printing.

In some embodiments, the ink cartridges of the inkjet printer used to print an composition as described above onto an adhesive applicator article as described above can be loaded with two different compositions. For example, when two ink cartridges are used in the printer, one ink cartridge, e.g., the dark cyan cartridge, may be loaded with an composition having a greater concentration of a semi-permanent colorant and/or temporary colorant than the composition loaded into the second ink cartridge, e.g., the light cyan cartridge.

Also provided herein is a kit comprising the adhesive applicator article of any one of the above adhesive applicator article embodiments. In some embodiments, the adhesive applicator article is an adhesive applicator article with an occlusive patch. In some embodiments, the kit further comprises a skin priming composition (see, e.g., U.S. application Ser. No. 16/785,549). In some embodiments of the kit, a priming article, e.g., a towelette, may be provided with the skin priming composition. In some embodiments, the skin priming composition can be provided in the kit on a priming article, e.g., a towelette, premoistened with the skin priming composition. In some embodiments, the kit further includes instructions.

Also provided herein is a kit comprising the adhesive applicator article of any one of the above adhesive applicator article embodiments and a skin priming composition. In some embodiments, the adhesive applicator article is an adhesive applicator article with an occlusive patch. In some embodiments, the kit further includes a towelette. In some embodiments, the towelette is moistened with the skin priming composition. In some embodiments of the kit, a priming article, e.g., a towelette, may be provided with the skin priming composition. In some embodiments, the skin priming composition may be provided in the kit on a priming article, e.g., a towelette, premoistened with the skin priming composition. In some embodiments, the kit further includes instructions. In some embodiments, the kit further includes an aftercare composition as described herein.

Additional ink (temporary and/or semi-permanent colorant) may be added to each above kit to allow e.g., correction, personalization, recoloring, etc. of the semi-permanent tattoo by freehand application. In some embodiments, the extra ink can have a composition as described above, or as described in U.S. Pat. No. 10,143,641, which is incorporated by reference herein in its entirety.

Also provided herein is a kit comprising one or more compositions (e.g., one or more compositions described herein) and/or an inkjet printing cartridge containing the composition, and an applicator article without the composition printed on it, for printing a custom design using an inkjet printer, or freehand drawing a design onto the applicator prior to application by a user. In some embodiments, each composition has a different semi-permanent colorant. In some embodiments each semi-permanent colorant has a different color.

### Composition B

In some embodiments, the compositions provided herein include a semi-permanent colorant (e.g., a genipin derivative, lawsone, a lawsone derivative, or a combination thereof, e.g., any of the combinations described herein); a solvent; and a humectant. In some embodiments, the compositions provided herein include a semi-permanent colorant (e.g., a genipin derivative, lawsone, a lawsone derivative, or a combination thereof, e.g., any of the combinations described herein); a solvent; and a thickening agent. In some embodiments, the compositions provided herein include a semi-permanent colorant; a solvent; and a humectant. In some embodiments, the compositions provided herein include a semi-permanent colorant; a solvent; a thickening agent, and a humectant. In some embodiments, the composition further includes a film-forming agent. In some embodiments, the ink compositions provided herein include a semi-permanent colorant; a solvent; and a film-forming agent. In some embodiments, the ink compositions described herein can be suitable for application to skin using a pen-like applicator. International Application No. PCT/CA2021/050790 describes genipin-based compositions that can be suitable for inkjet printing and is incorporated herein by reference in its entirety.

In some embodiments, the semi-permanent colorant (e.g., a genipin derivative, lawsone, a lawsone derivative, or a combination thereof, e.g., any of the combinations described herein) is present in an amount of about 0.1% to about 25% w/w of the composition. For example, about 0.1% to about 24%, about 0.1% to about 22%, about 0.1% to about 20%, about 0.1% to about 18%, about 0.1% to about 16%, about 0.1% to about 14%, about 0.1% to about 12%, about 0.1% to about 10%, about 0.1% to about 8%, about 0.1% to about 6%, about 0.1% to about 4%, about 0.1% to about 2%, about 24% to about 25%, about 22% to about 25%, about 20% to about 25%, about 18% to about 25%, about 16% to about 25%, about 14% to about 25%, about 12% to about 25%, about 10% to about 25%, about 8% to about 25%, about 6% to about 25%, about 4% to about 25%, or about 2% to about 25% w/w of the composition. In some embodiments, the semi-permanent colorant is present in an amount of about 0.1% to about 3%, about 0.1% to about 1%, about 0.5% to about 1%, about 0.5% to about 1.5%, or about 0.5% to about 3% w/w of the composition. In some embodiments, the semi-permanent colorant is present in an amount of about 0.5% to about 1.5% w/w of the composition. In some embodiments, the semi-permanent colorant is present in an amount of about 5% to about 15%, about 10% to about 15%, about 10% to about 20%, about 15% to about 20%, about 8% to about 12%, about 10% to about 14%, about 12% to about 16%, about 14% to about 18%, or about 16% to about 20% w/w of the composition. In some embodiments, the semi-permanent colorant is present in an amount of about 12% to about 16% w/w of the composition. In some embodiments, the semi-permanent colorant is present in an amount of about 9% to about 11% w/w of the composition. In some embodiments, the semi-permanent colorant is present in an amount of about 0.5%, about 0.9%, about 1%, about 2%, about 3%, about 4%, about 4.5%, about 5%, about 5.5%, about 6%, about 6.5%, about 7%, about 7.5%, about 8%, about 8.5%, about 9%, about 9.5%, about 10%, about about 11%, about 11.5%, about 12%, about 12.5%, about 13%, about 13.5%, about 14%, about 15%, about 16%, about 17%, about 18%, about 19%, about 20%, about 21%, about 22%, about 23%, about 24%, or about 25% w/w of the composition. In some embodiments, the semi-permanent colorant is present in an amount of about 14% w/w of the composition. In some embodiments, the semi-permanent colorant is present in an amount of about 101% w/w of the composition. In some embodiments, the semi-permanent colorant comprises a genipin derivative, lawsone, a lawsone derivative, or a combination thereof.

In some embodiments, the solvent (e.g., any of the solvents described herein) is present in an amount of about 1% to about 95% w/w of the composition. For example, about 10% to about 95%, about 20% to about 95%, about 30% to about 95%, about 40% to about 95%, about 50% to about 95%, about 60% to about 95%, about 70% to about 95%, about 80% to about 95%, about 1% to about 20%, about 1 to about 30%, about 1% to about 40%, about 1% to about 50%, about 1% to about 60%, about 1% to about 70%, about 1% to about 80%, or about 1% to about 90% w/w of the composition. In some embodiments, the solvent is present in an amount of about 50% to about 75%, about 55% to about 80%, about 60% to about 85%, about 65% to about 90%, about 55% to about 75%, about 60% to about 80%, about 65% to about 85%, about 70% to about 90%, about 75% to about 95%, about 60% to about 70%, about 65% to about 75%, about 70% to about 80%, or about 75% to about 85% w/w of the composition. For example, about 60% to about 65%, about 62% to about 67%, about 65% to about 70%, about 68% to about 72%, about 70% to about 75%, about 72% to about 76%, about 75% to about 80%, about 77% to about 82%, about 80% to about 85%, about 82% to about 87%, about 85% to about 90%, about 87% to about 92%, or about 90% to about 95% w/w of the composition. In some embodiments, the solvent is present in an amount of about 80% to about 90% w/w of the composition. In some embodiments, the solvent is present in an amount of about 76% to about 79% w/w of the composition. In some embodiments, the solvent is present in an amount of about 77% to about 78% w/w of the composition. In some embodiments, the solvent is present in an amount of about 72% to about 76% w/w of the composition. In some embodiments, the solvent is present in an amount of about 69% to about 71% w/w of the composition. In some embodiments, the solvent is present in an amount of about 70% to about 71% w/w of the composition. In some embodiments, the solvent is present in an amount of about 50%, about 55%, about 60%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, or about 95% w/w of the composition. In some embodiments, the solvent is present in an amount of about 74% w/w of the composition. In some embodiments, the solvent is present in an amount of about 77% w/w of the composition. In some embodiments, the solvent is present in an amount of about 71% w/w of the composition. In some embodiments, the solvent is present in an amount of about 86% w/w of the composition.

In some embodiments, the solvent is a combination of two or more solvents, e.g., two or more of water, methanol, ethanol, isopropanol, and diethylene glycol monoethyl ether. In some embodiments, each solvent is independently present in an amount of about 5% to about 80% w/w of the composition. For example, each solvent can be independently present in an amount of about 5% to about 10%, about 5% to about 20%, about 5% to about 30%, about 5% to about 40%, about 5% to about 50%, about 5% to about 60%, about 5% to about 70%, about 70% to about 80%, about 60% to about 80%, about 50% to about 80%, about 40% to about 80%, about 30% to about 80%, about 20% to about 80%, or about 10% to about 80% w/w of the composition. In some embodiments, the solvent is independently present in an amount of 10% to about 20%, about 15% to about 25%, about 20% to about 30%, about 25% to about 35%, about 30% to about 40%, about 35% to about 45%, about 40% to about 50%, about 45% to about 50%, about 50% to about 60%, or about 65% to about 75% w/w of the composition. For example, each solvent can be independently present in an amount of about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 60%, about 65%, about 70%, or about 72%, w/w of the composition.

In some embodiments, the solvent is a combination of water and ethanol. In some embodiments, the water is present in an amount of about 15% to about 40% w/w of the composition and the ethanol is present in an amount of about 30% to about 55% w/w of the composition. For example, the water is present in an amount of about 20% to about 30%, about 25% to about 35%, about 25% to about 30%, or about 28% to about 32% w/w of the composition and the ethanol is present in an amount of about 35% to about 45%, about 40% to about 50%, about 40% to about 55%, about 45% to about 55%, about 45% to about 50%, about 40% to about 45%, about 42% to about 47%, or about 45% to about 50% w/w of the composition. In some embodiments, the water is present in an amount of about 25% to about 30% w/w of the composition and the ethanol is present in an amount of about 40% to about 50% w/w of the composition. In some embodiments, the water is present in an amount of about 28% to about 32% w/w of the composition and the ethanol is present in an amount of about 45% to about 50% w/w of the composition. In some embodiments, the water is present in an amount of about 15% to about 25%, about 18% to about 22%, or about 19% to about 21% w/w of the composition and the ethanol is present in an amount of about 45% to about 55%, about 48% to about 52%, or about 49% to about 51% w/w of the composition. In some embodiments, the water is present in an amount of about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%, w/w of the composition and the ethanol is present in an amount of about 40%, about 41%, about 42%, about 43%, about 44%, about 45%, about 46%, about 47%, about 48%, about 49%, about 50%, about 51%, about 52%, about 53%, about 54%, or about 55% w/w of the composition. In some embodiments, the water is present in an amount of about 28% w/w of the composition and the ethanol is present in an amount of about 46% w/w of the composition.

In some embodiments, the water is present in an amount of about 65% to about 75% w/w of the composition and the ethanol is present in an amount of about 10% to about 20% w/w of the composition. In some embodiments, the water is present in an amount of about 72% w/w of the composition and the ethanol is present in an amount of about 14% w/w of the composition. In some embodiments, the water is present in an amount of about 30% w/w of the composition and the ethanol is present in an amount of about 48% w/w of the composition. In some embodiments, the water is present in an amount of about 21% w/w of the composition and the ethanol is present in an amount of about 50% w/w of the composition.

As used herein, a “thickening agent” refers to an agent that increases the viscosity of a liquid. In some embodiments, the thickening agent increases the viscosity of the liquid without substantially changing other properties of the liquid. Non-limiting examples of thickening agents include starches, gums (e.g., natural and synthetic gums), cellulosics, and arabinogalactan. Non-limiting examples of starches include aluminum starch octenylsuccinate. Non-limiting examples of gums include xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, and pullulan. Non-limiting examples of cellulosics include hydroxyethylcellulose, hydroxypropyl cellulose, hydroxypropyl methylcellulose, ethylcellulose, soybean hemicellulose, and sodium carboxy methylcellulose.

In some embodiments, the thickening agent is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments, the thickening agent is present in an amount of about 1% to about 5% w/w of the composition. In some embodiments, the thickening agent is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition. In some embodiments, the thickening agent is present in an amount of about 4% w/w of the composition.

In some embodiments, the thickening agent is present in an amount of about 0.01% to about 1% w/w of the composition. For example about 0.01% to about 0.05%, about 0.01% to about 0.1%, about 0.01% to about 0.15%, about 0.01% to about 0.2%, about 0.01% to about 0.3%, about 0.01% to about 0.4%, about 0.01% to about 0.5%, about 0.01% to about 0.6%, about 0.01% to about 0.7%, about 0.01% to about 0.8%, about 0.01% to about 0.9%, about 0.9% to about 1%, about 0.8% to about 1%, about 0.7% to about 1%, about 0.6% to about 1%, about 0.5% to about 1%, about 0.4% to about 1%, about 0.3% to about 1%, about 0.2% to about 1%, about 0.15% to about 1%, about 0.1% to about 1%, or about 0.05% to about 1% w/w of the composition. In some embodiments, the thickening agent is present in an amount of about 0.05% to about 0.25%, about 0.05% to about 0.2%, about 0.1% to about 0.2%, or about 0.1% to about 0.15% w/w of the composition. In some embodiments, the thickening agent is present in an amount of about 0.05% to about 0.2% w/w of the composition. In some embodiments, the thickening agent is present in an amount of about 0.01%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.2%, about 0.22%, about 0.24%, about 0.26%, about 0.28%, about 0.3%, about 0.4%, about 0.5%, about 0.6%, about 0.8%, about or about 1% w/w of the composition. In some embodiments, the thickening agent is present in an amount of about 0.12% w/w of the composition.

In some embodiments, the thickening agent is xanthan gum. In some embodiments, the xanthan gum is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments, the xanthan gum is present in an amount of about 1% to about 5% w/w of the composition. In some embodiments, the xanthan gum is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition. In some embodiments, the xanthan gum is present in an amount of about 4% w/w of the composition.

In some embodiments, the thickening agent is a combination of two or more thickening agents, e.g., two or more of arabinogalactan, aluminum starch octenylsuccinate, xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, pullulan, hydroxyethylcellulose, hydroxypropyl cellulose, hydroxypropyl methylcellulose, ethylcellulose, soybean hemicellulose, and sodium carboxy methylcellulose. In some embodiments, each thickening agent is independently present in an amount of about 0.01% to about 0.5% w/w of the composition. For example, each thickening agent can be independently present in an amount of about 0.01% to about 0.05%, about 0.01% to about 0.1%, about 0.01% to about 0.15%, about 0.01% to about 0.2%, about 0.01% to about 0.25%, about 0.01% to about 0.3%, about 0.01% to about 0.35% w/w of the composition. about 0.01% to about 0.4%, about 0.01% to about 0.45%, about 0.45% to about 0.5%, about 0.4% to about 0.5%, about 0.35% to about 0.5%, about 0.3% to about 0.5%, about 0.25% to about 0.5%, about 0.2% to about 0.5%, about 0.15% to about 0.5%, about 0.1% to about 0.15%, or about 0.05% to about 0.15% w/w of the composition. In some embodiments, each thickening agent is independently present in an amount of about 0.1% to about 0.04%, about 0.02% to about 0.06%, about 0.03% to about 0.07%, 0.04% to about 0.08%, about 0.06% to about 0.1%, or about 0.08% to about 0.12%, or about 0.1% to about 0.14% w/w of the composition. For example, each thickening agent can be independently present in an amount of about 0.01%, about 0.02%, about about 0.04%, about 0.05%, about 0.06%, about 0.07%, about 0.08%, about about 0.1%, about 0.11%, about 0.12%, about 0.13%, about 0.14%, about 0.15%, about 0.16%, about 0.17%, about 0.18%, about 0.19%, about 0.2%, about about 0.24%, about 0.26%, about 0.28%, about 0.3%, about 0.4%, about about 0.6%, about 0.8%, about 0.9% or about 1% w/w of the composition.

In some embodiments, the thickening agent is a combination of hydroxyethylcellulose and xanthan gum. In some embodiments, the hydroxyethylcellulose is present in an amount of about 0.01% to about 0.5% w/w of the composition and xanthan gum is present in an amount of about 0.01% to about 0.5% w/w of the composition. In some embodiments, the hydroxyethylcellulose is present in an amount of about 0.01% to about 0.04%, about 0.02% to about 0.06%, about 0.03% to about 0.07%, 0.04% to about 0.08%, about 0.06% to about 0.1%, or about 0.08% to about 0.12%, or about 0.1% to about 0.14% w/w of the composition and xanthan gum is present in an amount of about about 0.01% to about 0.04%, about 0.02% to about 0.06%, about 0.03% to about 0.07%, 0.04% to about 0.08%, about 0.06% to about 0.1%, or about 0.08% to about 0.12%, or about 0.1% to about 0.14% w/w of the composition. In some embodiments, the hydroxyethylcellulose is present in an amount of about 0.04% to about 0.08% w/w of the composition and xanthan gum is present in an amount of about 0.04% to about 0.08% w/w of the composition. In some embodiments, the hydroxyethylcellulose is present in an amount of about about 0.02%, about 0.03%, about 0.04%, about 0.05%, about 0.06%, about about 0.08%, about 0.09%, about 0.1%, about 0.11%, about 0.12%, about about 0.14%, about 0.15%, about 0.16%, about 0.17%, about 0.18%, about or about 0.2% w/w of the composition and xanthan gum is present in an amount of about 0.01%, about 0.02%, about 0.03%, about 0.04%, about 0.05%, about about 0.07%, about 0.08%, about 0.09%, about 0.1%, about 0.11%, about about 0.13%, about 0.14%, about 0.15%, about 0.16%, about 0.17%, about about 0.19%, or about 0.2% w/w of the composition. In some embodiments, the hydroxyethylcellulose is present in an amount of about 0.06% w/w of the composition and the xanthan gum is present in an amount of about 0.06% w/w of the composition.

A “humectant” as used herein refers to a substance that attracts water. For example, a humectant may attract water to bring moisture to the skin. Non-limiting examples of humectants include polyhydric alcohols, for example, polyalkylene glycols (e.g., alkylene polyols and their derivatives), alpha hydroxy acids, sugars, Aloe vera gel, vegetable oil, lithium chloride, allantoin, urea, and dicyanamide. Non-limiting examples of polyhydric alcohols include glycerol, lactic acid, propylene glycol, propanediol, polyethylene glycol, dipropylene glycol, polypropylene glycol, polyethylene glycol and derivatives thereof, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid.

In some embodiments, the humectant is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the humectant is present in an amount of about 0.5% to about 2%, about 0.5% to about 5%, about 1% to about 10%, about 1% to about 5%, about 2% to about 7%, about 5% to about 10%, about 7% to about 12%, or about 10% to about 15% w/w of the composition. In some embodiments, the humectant is present in an amount of about 0.5% to about 1.5% w/w of the composition. In some embodiments, the humectant is present in an amount of about 2% to about 6% w/w of the composition. In some embodiments, the humectant is present in an amount of about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the humectant is present in an amount of about 4% w/w of the composition. In some embodiments, the humectant is present in an amount of about 1% w/w of the composition.

In some embodiments, the humectant is propanediol. In some embodiments, the propanediol is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the propanediol is present in an amount of about 0.5% to about 2%, about 0.5% to about 5%, about 1% to about 10%, about 1% to about 5%, about 2% to about 7%, about 5% to about 10%, about 7% to about 12%, or about 10% to about 15% w/w of the composition. In some embodiments, the propanediol is present in an amount of about 0.5% to about 1.5% w/w of the composition. In some embodiments, the propanediol is present in an amount of about 2% to about 6% w/w of the composition. In some embodiments, the propanediol is present in an amount of about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the propanediol is present in an amount of about 4% w/w of the composition. In some embodiments, the propanediol is present in an amount of about 1% w/w of the composition.

In some embodiments, the humectant is glycerin. In some embodiments, the glycerin is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the glycerin is present in an amount of about 1% to about 10%, about 1% to about 5%, about 2% to about 7%, about 5% to about 10%, about 7% to about 12%, or about 10% to about 15% w/w of the composition. In some embodiments, the glycerol is present in an amount of about 2% to about 6% w/w of the composition. In some embodiments, the glycerol is present in an amount of about 1%, about 1.5%, about 2%, about 2.5%, about 3%, about 3.5%, about 4%, about 4.5%, about 5%, about about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the glycerol is present in an amount of about 4% w/w of the composition.

In some embodiments, the humectant is vegetable oil. In some embodiments, the vegetable oil is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the vegetable oil is present in an amount of about 1% to about 10%, about 1% to about 5%, about 2% to about 7%, about 5% to about 10%, about 7% to about 12%, or about 10% to about 15% w/w of the composition. In some embodiments, the vegetable oil is present in an amount of about 7% to about 11% w/w of the composition. In some embodiments, the vegetable oil is present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the vegetable oil is present in an amount of about 9% w/w of the composition.

In some embodiments, the composition further includes a film-forming agent. As used herein, a “film-forming agent” refers to a compound that can produce a continuous film on skin. In some embodiments, a film-forming agent is a compound that produces a continuous film on skin upon drying. Non-limiting examples of film-forming agents include volatile silicone resins, polyvinylpyrrolidone, acrylates, acrylamides, copolymers, and isododecane resins. Non-limiting examples of volatile silicone resins include polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, and polysilicone-14. Non-limiting examples of copolymers include acrylates copolymer, styrene/acrylates copolymer, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, and polyester-10.

In some embodiments, the film-forming agent is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the film-forming agent is present in an amount of about 1% to about 10%, about 1% to about 5%, about 2% to about 7%, about 5% to about 10%, about 7% to about 12%, or about 10% to about 15% w/w of the composition. In some embodiments, the film-forming agent is present in an amount of about 4% to about 6% w/w of the composition. In some embodiments, the film-forming agent is present in an amount of about 6% to about 9% w/w of the composition. In some embodiments, the film-forming agent is present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the film-forming agent is present in an amount of about 5% w/w of the composition. In some embodiments, the film-forming agent is present in an amount of about 7.5% w/w of the composition.

In some embodiments, the film-forming agent is a combination of two or more film-forming agents. For example, two or more of polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates copolymer, styrene/acrylates copolymer, polyvinylpyrrolidone, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, and polyester-10. In some embodiments, each film-forming agent is independently present in an amount of about 0.1% to about 15% w/w of the composition. For example, about 0.1% to about 1%, about 0.1% to about 5%, about 0.1% to about 10%, about 10% to about 15%, about 5% to about 15%, or about 1% to about 15% w/w of the composition. In some embodiments, each film-forming agent is present in an amount of about 0.1% to about 2%, about 0.5% to about 2%, about 1% to about 10%, about 1% to about 5%, about 2% to about 7%, about 5% to about 10%, about 7% to about 12%, or about 10% to about 15% w/w of the composition. In some embodiments, the film-forming agent is present in an amount of about 1% to about 10%, about 1% to about 5%, about 2% to about 7%, about 5% to about 10%, about 7% to about 12%, or about 10% to about 15% w/w of the composition. For example, about 0.1%, about 0.25%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition.

In some embodiments, the film-forming agent is a combination of polymethylsilsesquioxane and acrylates/C12-22 alkyl methacrylate copolymer. In some embodiments, the polymethylsilsesquioxane is present in an amount of about 0.5% to about 10% w/w of the composition and the acrylates/C12-22 alkyl methacrylate copolymer is present in an amount of about 0.5% to about 10% w/w of the composition. For example, the polymethylsilsesquioxane is present in an amount of about 0.05% to about 1%, about 0.05% to about 5%, about 0.05% to about 8%, about 8% to about 10%, about 5% to about 10%, about 1% to about 10%, or about 1% to about 5% w/w of the composition and the acrylates/C12-22 alkyl methacrylate copolymer is present in an amount of about 0.05% to about 1%, about 0.05% to about 5%, about 0.05% to about 8%, about 8% to about 10%, about 5% to about 10%, about 1% to about 10%, or about 1% to about 5% w/w of the composition. In some embodiments, the polymethylsilsesquioxane is present in an amount of about 1% to about 5% w/w of the composition and the acrylates/C12-22 alkyl methacrylate copolymer is present in an amount of about 1% to about 5% w/w of the composition. In some embodiments, the polymethylsilsesquioxane is present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition and the acrylates/C12-22 alkyl methacrylate copolymer is present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition. In some embodiments, the polymethylsilsesquioxane is present in an amount of about 2% w/w of the composition and the acrylates/C12-22 alkyl methacrylate copolymer is present in an amount of about 3% w/w of the composition.

In some embodiments, the film-forming agent is a combination of acrylates copolymer, polyvinylpyrrolidone, and styrene/acrylates copolymer. In some embodiments, the acrylates copolymer is present in an amount of about 0.1% to about 10% w/w of the composition; the polyvinylpyrrolidone is present in an amount of about 0.1% to about 10% w/w of the composition; and the styrene/acrylates copolymer is present in an amount of about 0.1% to about 10% w/w of the composition. In some embodiments, the acrylates copolymer is present in an amount of about 0.1% to about 1%, about 0.1% to about 5%, about 0.1% to about 8%, about 8% to about 10%, about 5% to about 10%, or about 1% to about 10% w/w of the composition; the polyvinylpyrrolidone is present in an amount of about 0.1% to about 1%, about 0.1% to about 5%, about 0.1% to about 8%, about 8% to about 10%, about 5% to about 10%, or about 1% to about 10% w/w of the composition; and the styrene/acrylates copolymer is present in an amount of about 0.1% to about 1%, about 0.1% to about 5%, about 0.1% to about 8%, about 8% to about 10%, about 5% to about 10%, or about 1% to about 10% w/w of the composition. In some embodiments, the acrylates copolymer is present in an amount of about 0.1% to about 1% w/w of the composition; the polyvinylpyrrolidone is present in an amount of about 0.5% to about 1.5% w/w of the composition; and the styrene/acrylates copolymer is present in an amount of about 5% to about 7% w/w of the composition. In some embodiments, the acrylates copolymer is present in an amount of about 0.1%, about 0.25%, about 0.5%, about 1%, about 1.5%, about 2%, about 2.5%, about 3%, about 3.5%, about 4%, about 4.5%, or about 5% w/w of the composition; the polyvinylpyrrolidone is present in an amount of about 0.25%, about 0.5%, about 1%, about 1.5%, about 2%, about 2.5%, about 3%, about 3.5%, about 4%, about 4.5%, about 5%, about 5.5%, or about 6% w/w of the composition; and the styrene/acrylates copolymer is present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition. In some embodiments, the acrylates copolymer is present in an amount of about 0.25% w/w of the composition; the polyvinylpyrrolidone is present in an amount of about 1% w/w of the composition; and the styrene/acrylates copolymer is present in an amount of about 6% w/w of the composition.

In some embodiments, the composition can further include an amine. Non-limiting examples of amines include: arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, 6-aminolevulinic acid, 4-aminobenzoic acid, and γ-aminobutyric acid.

In some embodiments, the amine is present in an amount of about 0.01% to about 1% w/w of the composition. For example, about 0.01% to about 0.05%, about 0.01% to about 0.1%, about 0.01% to about 0.5%, about 0.5% to about 1%, about 0.1% to about 1%, about 0.05% to about 1%, or about 0.1% to about 1% w/w of the composition. In some embodiments, the amine is present in an amount of about 0.02% w/w of the composition.

In some embodiments, the composition can further include a temporary colorant (e.g., any of the temporary colorants described herein). In some embodiments, the temporary colorant is ultramarine blue.

In some embodiments, the temporary colorant is present in an amount of less than about 0.01% w/w of the composition. In some embodiments, the temporary colorant is present in an amount of about 0.001% to about 0.01% w/w of the composition.

In some embodiments, the composition can further include a preservative (e.g., any of the preservatives described herein). In some embodiments, the preservative is present in an amount of about 0.05% to about 10% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 0.05% to about 8%, about 0.05% to about 9%, about 9% to about 10%, about 8% to about 10%, about 7% to about 10%, about 6% to about 10%, about 5% to about 10%, about 4% to about 10%, about 3% to about 10%, about 2% to about 10%, about 1% to about 10%, about 0.5% to about 10%, or about 0.1% to about 10% w/w of the composition. In some embodiments, the preservative is present in an amount of about 1% to about 5% w/w of the composition. In some embodiments, the preservative is present in an amount of 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, the preservative is present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about about 0.05% to about 0.2%, about 0.2% to about 0.5%, about 0.15% to about or about 0.1% to about 0.3% w/w of the composition. In some embodiments, the preservative is present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition. In some embodiments, the preservatives is present in an amount of about 3% w/w of the composition. In some embodiments, the preservatives is present in an amount of about 1% w/w of the composition.

In some embodiments, the preservative is a combination of two or more preservatives. For example, two or more of ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′43-(hydroxymethyl)-2,5-dioxoimidazolidin-4-yl]urea], polyaminopropyl biguanide, methenamine, quaternium-climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), sodium lignosulfonate, butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol. In some embodiments, each preservative is independently present in an amount of about 0.01% to about 8% w/w of the composition. For example, about 0.01% to about 0.05%, about 0.01% to about 0.1%, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 7% to about 8%, about 6% to about 8%, about 5% to about 8%, about 4% to about 8%, about 3% to about 8%, about 2% to about 8%, about 1% to about 8%, about 0.5% to about 8%, or about 0.1% to about 8% w/w of the composition. In some embodiments, each preservative is independently present in an amount of about 0.05% to about 8% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 7% to about 8%, about 6% to about 8%, about 5% to about 8%, about 4% to about 8%, about 3% to about 8%, about 2% to about 8%, about 1% to about 8%, about 0.5% to about 8%, or about 0.1% to about 8% w/w of the composition. In some embodiments, the each preservative is independently present in an amount of about 1% to about 5% w/w of the composition. In some embodiments, each preservative is independently present in an amount of about 0.025% to about 0.1%, about 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, each preservative is independently present in an amount of about 0.5% to about 1.5%, about 0.5% to about 1.0%, about 1.5% to about 2%, about 1% to about 2%, about 0.5% to about 2%, or about 0.8% to about 2% w/w of the composition. In some embodiments, each preservative is independently present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition.

In some embodiments, the preservative includes a combination of phenoxyethanol and ethylhexylglycerin. In some embodiments, the preservative further includes 1,2-hexanediol (e.g., the preservative is a combination of phenoxyethanol, ethylhexylglycerin, and 1,2-hexanediol). In some embodiments, the preservative further includes EDTA (e.g., the preservative is a combination of phenoxyethanol, ethylhexylglycerin, and EDTA). In some embodiments, the phenoxyethanol is present in an amount of about 0.1% and 5% w/w of the composition; the ethylhexylglycerin is present in an amount of about 0.01% to about 3% w/w of the composition; and the 1,2-hexanediol is present in an amount of about 0.1% to about 5% w/w of the composition. In some embodiments, the phenoxyethanol is present in an amount of about 0.1% to about 0.5%, about 0.1% to about 1%, about 0.1% to about 2%, about 0.1% to about 3%, about 0.1% to about 4%, about 4% to about 5%, about 3% to about 5%, about 2% to about 5%, about 1% to about 5%, about 0.5% to about 5%, about 0.5% to about 1%, about 0.5% to about 2.5%, about 1% to about 3%, about 1.5% to about 3.5%, about 2% to about 4%, or about 2.5% to about 4.5% w/w of the composition; the ethylhexylglycerin is present in an amount of about 0.01% to about 0.01% to about 0.5%, 0.01% to about 1%, about 0.01% to about 2%, about 2% to about 3%, about 1% to about 3%, about 0.1% to about 3%, about 0.05% to about 3%, about 0.05% to about 0.15%, about 0.05% to about 1%, 0.5% to about 2.5%, about 1% to about 3%, about 1.5% to about 3.5%, about 2% to about 4%, about 2.5% to about 4.5%, or about 3% to about 5% w/w of the composition; and the 1,2-hexanediol is present in an amount of about 0.1% to about 1%, about 0.1% to about 2%, about 0.1% to about 3%, about 0.1% to about 4%, about 4% to about 5%, about 3% to about 5%, about 2% to about 5%, about 1% to about 5%, 0.5% to about 2.5%, about 1% to about 3%, about 1.5% to about 3.5%, about 2% to about 4%, about 2.5% to about 4.5%, or about 3% to about 5% w/w of the composition. In some embodiments, the phenoxyethanol is present in an amount of about 0.5%, about 0.6%, about 0.7%, about 0.8%, about 0.9%, about 1%, about 1.1%, about 1.2%, about 1.3%, about 1.4%, about 1.5%, about 2%, about 2.5%, about 3%, about 3.5%, about 4%, or about 4.5% w/w of the composition; the ethylhexylglycerin is present in an amount of about 0.01%, about 0.05%, about 0.06%, about 0.07%, about 0.08%, about 0.09%, about 0.1%, about 0.12%, about 0.13%, about 0.14%, about 0.15%, about 0.25%, about 0.5%, about 1%, about 1.5%, about 2%, or about 2.5% w/w of the composition; and the 1,2-hexanediol is present in an amount of about 0.5%, about 1%, about 1.5%, about 2%, about 2.5%, about 3%, about 3.5%, about 4%, about 4.5%, about 5%, about 5.5%, or about 6% w/w of the composition.

In some embodiments, the preservative further includes EDTA (e.g., the preservative is a combination of phenoxyethanol, ethylhexylglycerin, and EDTA). In some embodiments, the phenoxyethanol is present in an amount of about 0.1% and 5% w/w of the composition; the ethylhexylglycerin is present in an amount of about 0.01% to about 3% w/w of the composition; and the EDTA is present in an amount of about 0.01% to about 2% w/w of the composition. In some embodiments, the phenoxyethanol is present in an amount of about 0.1% to about 0.5%, about 0.1% to about 1%, about 0.1% to about 2%, about 0.1% to about 3%, about 0.1% to about 4%, about 4% to about 5%, about 3% to about 5%, about 2% to about 5%, about 1% to about 5%, about 0.5% to about 5%, about 0.5% to about 1%, about 0.5% to about 2.5%, about 1% to about 3%, about 1.5% to about 3.5%, about 2% to about 4%, or about 2.5% to about 4.5% w/w of the composition; the ethylhexylglycerin is present in an amount of about 0.01% to about 0.1%, 0.01% to about 0.5%, 0.01% to about 1%, about 0.01% to about 2%, about 2% to about 3%, about 1% to about 3%, about 0.1% to about 3%, about 0.05% to about 3%, about 0.05% to about 0.15%, about 0.05% to about 1%, 0.5% to about 2.5%, about 1% to about 3%, about 1.5% to about 3.5%, about 2% to about 4%, about 2.5% to about 4.5%, or about 3% to about 5% w/w of the composition; and the EDTA is present in an amount of about 0.01% to about about 0.01% to about 0.1%, about 0.01% to about 0.5%, about 0.01% to about 1%, about 0.1% to about 1.5%, about 1.5% to about 2%, about 1% to about 2%, about 0.5% to about 2%, about 0.1% to about 2%, or about 0.05% to about 2% w/w of the composition. In some embodiments, the phenoxyethanol is present in an amount of about 0.5%, about 0.6%, about 0.7%, about 0.8%, about 0.9%, about 1%, about 1.1%, about 1.2%, about 1.3%, about 1.4%, about 1.5%, about 2%, about 2.5%, about 3%, about 3.5%, about 4%, or about 4.5% w/w of the composition; the ethylhexylglycerin is present in an amount of about 0.01%, about 0.05%, about 0.06%, about 0.07%, about 0.08%, about 0.09%, about 0.1%, about 0.12%, about 0.13%, about 0.14%, about 0.15%, about 0.25%, about 0.5%, about 1%, about 1.5%, about 2%, or about 2.5% w/w of the composition; and the EDTA is present in an amount of about 0.01%, about 0.05%, about 0.1%, about 0.5%, about 1%, about 1.5%, or about 2% w/w of the composition.

In some embodiments, the phenoxyethanol and ethylhexylglycerin are present in the composition at a ratio of about 9:1 phenoxyethanol:ethylhexylglycerin (e.g., EUXYL® PE9010).

In some embodiments, the EDTA is disodium EDTA.

In some embodiments, the preservative further includes sodium lignosulfonate.

In some embodiments, the ink composition can further include one or more temporary colorants. In some embodiments, the one or more temporary colorants are present in an amount of about 0.1% to about 10% w/w of the composition. For example, about 0.1% to about 1%, about 0.1% to about 5%, about 0.1% to about 8%, about 8% to about 10%, about 5% to about 10%, or about 1% to about 10% w/w of the composition. In some embodiments, the one or more temporary colorant are present in an amount of about 2% to about 3% w/w of the composition. In some embodiments, the one or more temporary colorant are present in an amount of about 2.5% w/w of the composition. In some embodiments, the one or more temporary colorants are selected from the group consisting of: iron oxide black (Fe3O4), iron oxide (FeO), carbon, logwood, ochre, cinnabar (HgS), cadmium red (CdSe), iron (III) oxide (Fe2O3), naphthol-AS pigment, disazodiarylide, disazopyrazolone, cadmium seleno-sulfide, cadmium yellow (CdS, CdZnS), curcuma yellow, chrome yellow (PbCrO4), disazodiarylide, chromium oxide (Cr2O3), malachite [Cu2(CO3)(OH)2], a ferrocyanide, a ferricyanide, lead chromate, monoazo pigment, Cu/Al phthalocyanine, Cu phthalocyanine, azure blue, cobalt blue, Cu-phthalocyanine, manganese violet (manganese ammonium pyrophosphate), an aluminum salt, quinacridone, dioxazine/carbazole, lead white (lead carbonate), titanium dioxide (TiO2), barium sulfate (BaSO4), zinc oxide, anthraquinone dyes and derivatives, annatto, caramel, β-carotene, bismuth citrate, disodium EDTA copper, potassium sodium copper chlorophyllin, dihydroxyacetone, bismuth oxychloride, guaiazulene, henna, ferric ammonium ferrocyanide, ferric ferrocyanide, chromium hydroxide green, chromium oxide greens, guanine, lead acetate, pyrophillite, mica, silver, titanium dioxide, aluminum powder, bronze powder, copper powder, ultramarines, manganese violet, zinc oxide, luminescent zinc sulfide, D&C Black Nos. 2 and 3, FD&C Blue No. 1 (e.g., acid blue 9), D&C Blue No. 4, D&C Brown No. 1, FD&C Green No. 3, D&C Green Nos. 5, 6, and 8, D&C Orange Nos. 4, 5, 10 and 11, FD&C Red Nos. 4, D&C Red Nos. 6, 7, 17, 21, 22, 27, 28, 30, 32, 33, 34, 36 and 40, D&C Violet No. 2, Ext. D&C Violet No. 2, FD&C Yellow Nos. 5 and 6, D&C Yellow Nos. 7, 8, 10 and 11, and Ext. D&C Yellow No. 7. In some embodiments, the temporary colorant does not contain an amine group. In some embodiments, the temporary colorant is D&C Black No. 2.

In some embodiments, a composition as provided herein includes:
a semi-permanent colorant (e.g., a genipin derivative, lawsone, a lawsone derivative, or a combination thereof, e.g., any of the combinations described herein) present in an amount of about 10% to about 20% w/w of the composition;
a solvent present in an amount of about 65% to about 80% w/w of the composition; and
a thickening agent present in an amount of about 0.01% to about 0.3% w/w of the composition. In some embodiments, the solvent is a combination of water and ethanol. In some such embodiments, the water is present in an amount of about 25% to about 30% w/w of the composition, and the ethanol is present in an amount of about 40% to about 50% w/w of the composition. In some embodiments, the thickening agent is a combination of hydroxyethylcellulose and xanthan gum. In some such embodiments, the hydroxyethylcellulose is present in an amount of about 0.01% to about 0.1% w/w of the composition, and the xanthan gum is present in an amount of about 0.01% to about 0.1% w/w of the composition.

In some embodiments, the composition further includes a humectant present in an amount of about 1% to about 10% w/w of the composition. In some embodiments, the humectant is glycerol.

In some embodiments, the composition further includes a film-forming agent present in an amount of about 0.1% to about 10% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition.

In some embodiments, the composition further includes a preservative present in an amount of about 0.1% to about 5% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 10% to about 20% w/w of the composition;
a solvent present in an amount of about 65% to about 80% w/w of the composition; and
a humectant present in an amount of about 1% to about 10% w/w of the composition. In some embodiments, the solvent is a combination of water and ethanol.

In some embodiments, the water is present in an amount of about 25% to about 30% w/w of the composition. In some embodiments, the ethanol is present in an amount of about 40% to about 50% w/w of the composition. In some embodiments, the humectant is glycerol.

In some embodiments, the composition further includes a thickening agent present in an amount of about 0.01% to about 0.3% w/w of the composition. In some embodiments, the thickening agent is a combination of hydroxyethylcellulose and xanthan gum. In some such embodiments, the hydroxyethylcellulose is present in an amount of about 0.01% to about 0.1% w/w of the composition, and the xanthan gum is present in an amount of about 0.01% to about 0.1% w/w of the composition.

In some embodiments, the composition further includes a film-forming agent present in an amount of about 0.1% to about 10% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition. In some embodiments, the composition further includes a humectant present in an amount of about 1% to about 10% w/w of the composition. In some embodiments, the humectant is glycerol.

In some embodiments, the composition further includes a preservative present in an amount of about 0.1% to about 5% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 12% to about 16% w/w of the composition;
a solvent present in an amount of about 72% to about 76% w/w of the composition; and
a thickening agent present in an amount of about 0.05% to about 0.20% w/w of the composition. In some embodiments, the solvent is a combination of water and ethanol. In some such embodiments, the water is present in an amount of about 25% to about 30% w/w of the composition, and the ethanol is present in an amount of about 45% to about 47% w/w of the composition. In some embodiments, the thickening agent is a combination of hydroxyethylcellulose and xanthan gum. In some such embodiments, the hydroxyethylcellulose is present in an amount of about 0.04% to about 0.08% w/w of the composition, and the xanthan gum is present in an amount of about 0.04% to about 0.08% w/w of the composition.

In some embodiments, the composition further includes a film-forming agent present in an amount of about 4% to about 6% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition.

In some embodiments, the composition further includes a humectant present in an amount of about 2% to about 6% w/w of the composition. In some embodiments, the humectant is glycerol.

In some embodiments, the composition further includes a preservative present in an amount of 2% to about 4% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 12% to about 16% w/w of the composition;
a solvent present in an amount of about 72% to about 76% w/w of the composition; and
a humectant present in an amount of about 2% to about 6% w/w of the composition. In some embodiments, the solvent is a combination of water and ethanol. In some such embodiments, the water is present in an amount of about 25% to about 30% w/w of the composition, and the ethanol is present in an amount of about 45% to about 47% w/w of the composition. In some embodiments, the humectant is glycerol.

In some embodiments, the composition further includes a thickening agent present in an amount of about 0.05% to about 0.20% w/w of the composition. In some embodiments, the thickening agent is a combination of hydroxyethylcellulose and xanthan gum. In some such embodiments, the hydroxyethylcellulose is present in an amount of about 0.04% to about 0.08% w/w of the composition, and the xanthan gum is present in an amount of about 0.04% to about 0.08% w/w of the composition.

In some embodiments, the composition further includes a film-forming agent present in an amount of about 4% to about 6% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition.

In some embodiments, the composition further includes a preservative present in an amount of 2% to about 4% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 14% w/w of the composition;
a solvent present in an amount of about 74% w/w of the composition; and
a thickening agent present in an amount of about 0.12% w/w of the composition. In some embodiments, the solvent is a combination of water and ethanol. In some such embodiments, the water is present in an amount of about 28% w/w of the composition, and the ethanol is present in an amount of about 46% w/w of the composition. In some embodiments, the thickening agent is a combination of hydroxyethylcellulose and xanthan gum. In some embodiments, the hydroxyethylcellulose is present in an amount of about 0.06% w/w of the composition, and the xanthan gum is present in an amount of about 0.06% w/w of the composition.

In some embodiments, the composition further includes a film-forming agent present in an amount of about 5% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 2% w/w of the composition, and the acrylates/C12-22 alkyl methacrylate copolymer is present in an amount of about 3% w/w of the composition. In some embodiments, the composition further includes a humectant present in an amount of about 4% w/w of the composition. In some embodiments, the humectant is glycerol.

In some embodiments, the composition further includes a preservative present in an amount of 3% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 1% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 2% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 14% w/w of the composition;
a solvent present in an amount of about 74% w/w of the composition; and
a humectant present in an amount of about 4% w/w of the composition. In some embodiments, the solvent is a combination of water and ethanol. In some such embodiments, the water is present in an amount of about 28% w/w of the composition, and the ethanol is present in an amount of about 46% w/w of the composition. In some embodiments, the humectant is glycerol.

In some embodiments, the composition further includes a thickening agent present in an amount of about 0.12% w/w of the composition. In some embodiments, the thickening agent is a combination of hydroxyethylcellulose and xanthan gum. In some such embodiments, the hydroxyethylcellulose is present in an amount of about w/w of the composition, and the xanthan gum is present in an amount of about w/w of the composition.

In some embodiments, the composition further includes a film-forming agent present in an amount of about 5% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 2% w/w of the composition, and the acrylates/C12-22 alkyl methacrylate copolymer is present in an amount of about 3% w/w of the composition.

In some embodiments, the composition further includes a preservative present in an amount of 3% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 1% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 2% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 5% to about 15% w/w of the composition;
a solvent present in an amount of about 65% to about 85% w/w of the composition; and
a film-forming agent present in an amount of about 5% to about 10% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 9% to about 11% w/w of the composition;
a solvent present in an amount of about 75% to about 80% w/w of the composition; and
a film-forming agent present in an amount of about 6% to about 9% w/w of the composition.

In some embodiments, the solvent is a combination of water and ethanol. In some such embodiments, the water is present in an amount of about 25% to about 35% w/w of the composition, and the ethanol is present in an amount of about 45% to about 50% w/w of the composition. In some embodiments, the film-forming agent is a combination of acrylates copolymer, polyvinylpyrrolidone, and styrene/acrylates copolymer.

In some embodiments, the composition further includes a humectant present in an amount of about 0.5% to about 1.5% w/w of the composition. In some embodiments, the humectant is propanediol.

In some embodiments, the composition further includes a preservative present in an amount of 0.5% to about 1.5% w/w of the composition. In some embodiments, the preservative includes a combination of phenoxyethanol and ethylhexylglycerin. In some embodiments, the preservative includes a combination of phenoxyethanol, ethylhexylglycerin, and EDTA. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the EDTA is present in an amount of about 0.05% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 10% w/w of the composition;
a solvent present in an amount of about 77% w/w of the composition; and
a film-forming agent present in an amount of about 7.5% w/w of the composition.

In some embodiments, the solvent is a combination of water and ethanol. In some such embodiments, the water is present in an amount of about 30% w/w of the composition, and the ethanol is present in an amount of about 48% w/w of the composition. In some embodiments, the film-forming agent is a combination of acrylates copolymer, polyvinylpyrrolidone, and styrene/acrylates copolymer.

In some embodiments, the composition further includes a humectant present in an amount of about 1% w/w of the composition. In some embodiments, the humectant is propanediol.

In some embodiments, the composition further includes a preservative present in an amount of about 1.05% w/w of the composition. In some embodiments, the preservative includes a combination of phenoxyethanol and ethylhexylglycerin. In some embodiments, the preservative includes a combination of phenoxyethanol, ethylhexylglycerin, and EDTA. In some such embodiments, the phenoxyethanol and ethylhexylglycerin are together present in the composition in an amount of about 1% w/w of the composition. In some such embodiments, the phenoxyethanol and ethylhexylglycerin are present in the composition at a ratio of about 9:1 phenoxyethanol:ethylhexylglycerin. In some such embodiments, the EDTA is present in an amount of about 0.05% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 0.1% to about 1.5% w/w of the composition;
a solvent present in an amount of about 80% to about 90% w/w of the composition; and
a humectant present in an amount of about 10% to about 15% w/w of the composition.

In some embodiments, the solvent is a combination of water and ethanol. In some such embodiments, the water is present in an amount of about 65% to about 75% w/w of the composition, and the ethanol is present in an amount of about 10% to about 20% w/w of the composition. In some embodiments, the humectant is vegetable oil.

In some embodiments, the composition further includes a thickening agent present in an amount of about 1% to about 10% w/w of the composition. In some embodiments, the thickening agent is xanthan gum. In some embodiments, the composition further comprises a temporary colorant present in an amount of less than about 0.1% w/w of the composition. In some embodiments, the composition further comprises an amine present in an amount of about 0.01% to about 1% w/w of the composition.

In some embodiments, a composition as provided herein includes:
a semi-permanent colorant (e.g., a genipin derivative, lawsone, a lawsone derivative, or a combination thereof, e.g., any of the combinations described herein) present in an amount of about 0.9% w/w of the composition;
a solvent present in an amount of about 86% w/w of the composition; and
a humectant present in an amount of about 9% w/w of the composition. In some embodiments, the solvent is a combination of water and ethanol. In some such embodiments, the water is present in an amount of about 71% w/w of the composition, and the ethanol is present in an amount of about 14% w/w of the composition. In some embodiments, the humectant is vegetable oil.

In some embodiments, the composition further includes a thickening agent present in an amount of about 4% w/w of the composition. In some embodiments, the thickening agent is xanthan gum. In some embodiments, the composition further comprises a temporary colorant present in an amount of less than about 0.1% w/w of the composition. In some embodiments, the composition further comprises an amine present in an amount of about 0.02% w/w of the composition.

Also provided herein are methods of manufacturing a composition, e.g., Composition B as described herein. For example, such methods can include i) combining a thickening agent with a first solvent to form a first solution; ii) combining genipin with a second solvent to form a second solution; and iii) combining the first solution and second solution to form a composition. In some embodiments, methods of manufacturing a composition as described herein include: i) combining a thickening agent and a humectant with a first solvent to form a first solution; ii) combining genipin and a film-forming agent (e.g., a first film-forming agent) with a second solvent to form a second solution; and iii) combining the first solution and second solution to form a composition. In some embodiments, the methods described herein further include: iv) adding a film-forming agent (e.g., a first or second film-forming agent) agent and/or a preservative to the composition.

In some embodiments, the first solvent is any of the solvents described herein. In some embodiments, the second solvent is any of the solvents described herein. In some embodiments, the first solvent and second solvent are different.

In some embodiments, the first solvent is water. In some embodiments, the second solvent is ethanol.

In some embodiments, the thickening agent (e.g., any of the thickening agents described herein) and first solvent are present in a ratio of about 1:500 to about 4:500 thickening agent:first solvent in the first solution. In some embodiments, the thickening agent and first solvent are present in a ratio of about 2:500 thickening agent:first solvent in the first solution.

In some embodiments, the thickening agent is a combination of hydroxyethylcellulose and xanthan gum. In some embodiments, the combination of hydroxyethylcellulose and xanthan gum is present at a ratio of about 2:1 to about 1:2 hydroxyethylcellulose:xanthan gum. In some embodiments, the combination of hydroxyethylcellulose and xanthan gum is present at a ratio of about 1:1 hydroxyethylcellulose:xanthan gum.

In some embodiments, the thickening agent, humectant (e.g., any of the humectants described herein), and first solvent are present in a ratio of about 1:15:234 to about 1:62:187 thickening agent:humectant:first solvent in the first solution. In some embodiments, the thickening agent and first solvent are present in a ratio of about 1:31:218 thickening agent:humectant:first solvent in the first solution.

In some embodiments, the humectant is glycerin.

In some embodiments, the first solution is incubated at a temperature of about 70° C. to about 120° C. For example, about 70° C. to about 75° C., about 70° C. to about 80° C., about 70° C. to about 85° C., about 70° C. to about 90° C., about 70° C. to about 95° C., about 70° C. to about 100° C., about 70° C. to about 105° C., about 70° C. to about 110° C., about 70° C. to about 115° C., about 115° C. to about 120° C., about 110° C. to about 120° C., about 105° C. to about 120° C., about 100° C. to about 120° C., about 95° C. to about 120° C., about 90° C. to about 120° C., about 85° C. to about 120° C., about 80° C. to about 120° C., or about 75° C. to about 120° C. In some embodiments, the first solution is incubated at a temperature of about 80° C. to about 105° C., about 85° C. to about 100° C., or about 90° C. to about 95° C.

In some embodiments, the first solution is stirred.

In some embodiments, the first solution is incubated and/or stirred until the thickening agent is dissolved.

In some embodiments, the genipin and second solvent are present in a ratio of about 4:25 to about 3:5 genipin:second solvent in the second solution. In some embodiments, the genipin and second solvent are present in a ratio of about 3:10 genipin:second solvent in the second solution.

In some embodiments, the genipin, first film-forming agent (e.g., any of the film-forming agents described herein), and second solvent are present in a ratio of about 57:16:177 to about 57:4:189 genipin:film-forming agent:second solvent in the second solution. In some embodiments, the genipin, first film-forming agent, and second solvent are present in a ratio of about 57:8:185 genipin:film-forming agent:second solvent in the second solution. In some embodiments, the first film-forming agent is polymethylsilsesquioxane.

In some embodiments, the second solution is incubated at a temperature of about 30° C. to about 60° C. For example, about 30° C. to about 35° C., about 30° C. to about 40° C., about 30° C. to about 45° C., about 30° C. to about 50° C., about 30° C. to about 55° C., about 55° C. to about 60° C., about 50° C. to about 60° C., about 45° C. to about 60° C., about 40° C. to about 60° C., or about 35° C. to about 60° C. In some embodiments, the second solution is incubated at a temperature of about 35° C. to about 55° C., about 35° C. to about 45° C., about 40° C. to about 50° C., or about 45° C. to about 55° C.

In some embodiments, the second solution is stirred.

In some embodiments, the second solution is incubated and/or stirred until the genipin is dissolved.

In some embodiments, the temperature of the first solution is cooled. In some embodiments, the temperature of the first solution is cooled to about 20° C. to about 50° C. For example, about 20° C. to about 25° C., about 20° C. to about 30° C., about 20° C. to about 35° C., about 20° C. to about 40° C., about 20° C. about 45° C., about 45° C. to about 50° C., about 40° C. to about 50° C., about 35° C. to about 50° C., about 30° C. to about 50° C., or about 25° C. to about 50° C. In some embodiments, the temperature of the first solution is cooled to about 35° C. to about 45° C. For example, about 35° C., about 36° C., about 37° C., about 38° C., about 39° C., about 40° C., about 41° C., about 42° C., about 43° C., about 44° C., or about 45° C.

In some embodiments, the temperature of the second solution is cooled. In some embodiments, the temperature of the second solution is cooled to about 20° C. to about 50° C. For example, about 20° C. to about 25° C., about 20° C. to about 30° C., about 20° C. to about 35° C., about 20° C. to about 40° C., about 20° C. about 45° C., about 45° C. to about 50° C., about 40° C. to about 50° C., about 35° C. to about 50° C., about 30° C. to about 50° C., or about 25° C. to about 50° C. In some embodiments, the temperature of the second solution is cooled to about 35° C. to about 45° C. For example, about 35° C., about 36° C., about 37° C., about 38° C., about 39° C., about 40° C., about 41° C., about 42° C., about 43° C., about 44° C., or about 45° C.

In some embodiments, the first solution and second solution are present in a ratio of about 1:4 to about 1:1 first solution:second solution in the composition. In some embodiments, the first solution and second solution are present in a ratio of about 1:2 first solution:second solution in the composition.

In some embodiments, combining the first solution and second solution to form a composition includes stirring. In some embodiments, the composition is stirred until the composition is clear without any visible suspended particles.

In some embodiments, the composition is cooled to about room temperature. In some embodiments, the composition is cooled to about 20° C. to about 30° C. In some embodiments, the composition is cooled to about 25° C.

In some embodiments, the composition is cooled prior to the adding a film-forming agent (e.g., a first or second film-forming agent) agent and/or a preservative to the composition. In some embodiments, adding a film-forming agent (e.g., a first or second film-forming agent) agent and/or a preservative to the composition further comprises stirring the composition.

In some embodiments, the film-forming agent (e.g., any of the film-forming agents described herein) is present in an amount of about 1% to about 10% w/w of the composition. In some embodiments, the film-forming agent is present in an amount of about 2% to about 6% w/w of the composition. In some embodiments, the film-forming agent is selected from the group consisting of: polymethylsilsesquioxane, acrylates/C12-22 alkyl methacrylate copolymer, and a combination thereof.

In some embodiments, the second film-forming agent (e.g., any of the film-forming agents described herein) is present in an amount of about 1% to about 5% w/w of the composition. In some embodiments, the second film-forming agent is present in an amount of about 3% w/w of the composition. In some embodiments, the first film-forming agent and the second film-forming agent are different.

In some embodiments, the second film-forming agent is acrylates/C12-22 alkyl methacrylate copolymer.

In some embodiments, the preservative (e.g., any of the preservatives described herein) is present in an amount of about 0.1% to about 5% w/w of the composition. In some embodiments, the preservative is present in an amount of about 2% to about 4% w/w of the composition. In some embodiments, the preservative is present in an amount of about 3% w/w of the composition.

In some embodiments, the preservative comprises a combination of phenoxyethanol and ethylhexylglycerin. In some embodiments, phenoxyethanol and ethylhexylglycerin are present in the composition at a ratio of about 9:1 phenoxyethanol:ethylhexylglycerin. In some embodiments, the combination of phenoxyethanol and ethylhexylglycerin is present in an amount of about 0.1% to about 2% w/w of the composition. In some embodiments, the combination of phenoxyethanol and ethylhexylglycerin is present in an amount of about 1% w/w of the composition.

In some embodiments, the preservative comprises 1,2-hexanediol. In some embodiments, the 1,2-hexanediol is present in an amount of about 1% to about 3% w/w of the composition. In some embodiments, the 1,2-hexanediol is present in an amount of about 2% w/w of the composition.

In some embodiments, methods of manufacturing an ink composition as described herein include: i) combining genipin with a first solvent to form a genipin solution; ii) combining a film-forming agent (e.g., a first film-forming agent) with a second solvent to form a solution (e.g., a first solution or a subsequent solution); and iii) combining the genipin solution and a solution (e.g., a first solution or a subsequent solution) to form an ink composition. In some embodiments, the methods described herein can further include adding one or more of a film-forming agent (e.g., a second or third film-forming agent), a humectant (e.g., a first humectant), a preservative (e.g., a first preservative), and a temporary colorant (e.g., a first temporary colorant) to a genipin solution, a solution (e.g., a first solution or a subsequent solution), or the ink composition. In some embodiments, the methods described herein include adding one or more of a film-forming agent (e.g., a second or third film-forming agent), a humectant (e.g., a first humectant), and a preservative (e.g., a first preservative) to the solution (e.g., a first solution) to form a second solution (or a third solution, a fourth solution, etc., or a subsequent solution).

In some embodiments, the first solvent is any of the solvents described herein. In some embodiments, the second solvent is any of the solvents described herein. In some embodiments, the first solvent and second solvent are different.

In some embodiments, the first solvent comprises ethanol. In some embodiments, the first solvent comprises ethanol and water. In some embodiments, the second solvent comprises water.

In some embodiments, the genipin and first solvent are present in a ratio of about 4:25 to about 3:5 genipin:first solvent in the genipin solution. In some embodiments, the genipin and first solvent are present in a ratio of about 1:5 genipin:first solvent in the genipin solution.

In some embodiments, the genipin solution is stirred. In some embodiments, the genipin solution is stirred until the genipin is dissolved.

In some embodiments, the film-forming agent (e.g., a first film-forming agent) and second solvent are present in a ratio of about 1:40 to about 1:120 film-forming agent:second solvent in the solution. In some embodiments, the film-forming agent and second solvent are present in a ratio of about 1:80 film-forming agent:second solvent in the solution.

In some embodiments, the film-forming agent (e.g., a first film-forming agent) is any of the film-forming agents described herein. In some embodiments, the film-forming agent comprises acrylates copolymer.

In some embodiments, the solution (e.g., the first solution) is stirred. In some embodiments, the solution (e.g., the first solution) is stirred until the film-forming agent (e.g., a first film-forming agent) is dispersed.

In some embodiments, a second film-forming agent is combined with the first solution to form a second solution. In some embodiments, the second film-forming agent and first solution are present in a ratio of about 1:10 to about 1:40 second film-forming agent:first solution in the second solution. In some embodiments, the second film-forming agent and first solution are present in a ratio of about 1:21 second film-forming agent:first solution in the second solution.

In some embodiments, the second film-forming agent is any of the film-forming agents described herein. In some embodiments, the second film-forming agent comprises polyvinylpyrrolidone.

In some embodiments, the second solution is stirred.

In some embodiments, a third film-forming agent is combined with the second solution to form a third solution. In some embodiments, the third film-forming agent and second solution are present in a ratio of about 1:2 to about 1:10 third film-forming agent:second solution in the third solution. In some embodiments, the third film-forming agent and second solution are present in a ratio of about 3:11 third film-forming agent:second solution in the third solution.

In some embodiments, the third film-forming agent is any of the film-forming agents described herein. In some embodiments, the third film-forming agent comprises styrene/acrylates copolymer.

In some embodiments, the third solution is stirred.

In some embodiments, a humectant is combined with a solution (e.g., a first solution, a second solution, or a third solution) to form a fourth solution. In some embodiments, a humectant is combined with the third solution to form a fourth solution. In some embodiments, the humectant and solution (e.g., third solution) are present in a ratio of about 1:10 to about 1:40 humectant:solution in the fourth solution. In some embodiments, the humectant and solution (e.g., third solution) are present in a ratio of about 1:28 humectant:solution in the fourth solution.

In some embodiments, the humectant is any of the humectants described herein. In some embodiments, the humectant comprises propanediol.

In some embodiments, the fourth solution is stirred.

In some embodiments, a preservative is combined with a solution (e.g., a first solution, a second solution, a third solution, or a fourth solution) to form a fifth solution. In some embodiments, a preservative is combined with the fourth solution to form a fifth solution. In some embodiments, the preservative and solution (e.g., fourth solution) are present in a ratio of about 1:10 to about 1:40 preservative:solution in the fifth solution. In some embodiments, the preservative and solution (e.g., fourth solution) are present in a ratio of about 1:28 preservative:solution in the fifth solution.

In some embodiments, the preservative is any of the preservatives described herein. In some embodiments, the preservative comprises a combination of phenoxyethanol and ethylhexylglycerin. In some embodiments, the preservative comprises a combination of phenoxyethanol ethylhexylglycerin, and EDTA. In some embodiments, the phenoxyethanol and ethylhexylglycerin are combined with the solution (e.g., a fourth solution) before the EDTA is combined with the solution. In some embodiments, the solution (e.g., the fourth solution) is stirred between combining the phenoxyethanol and ethylhexylglycerin with the solution (e.g., the fourth solution) and combining the EDTA with the solution (e.g., the fourth solution).

In some embodiments, phenoxyethanol and ethylhexylglycerin are present in the composition at a ratio of about 9:1 phenoxyethanol:ethylhexylglycerin.

In some embodiments, the fifth solution is stirred.

In some embodiments, the genipin solution and solution (e.g., a first solution, a second solution, a third solution, a fourth solution, or a fifth solution) are present in a ratio of about 1:6 to about 1:1 genipin solution:solution in the ink composition. In some embodiments, the genipin solution and solution (e.g., a first solution, a second solution, a third solution, a fourth solution, or a fifth solution) are present in a ratio of about 1:2 genipin solution:solution in the ink composition.

In some embodiments, combining the genipin solution and second solution to form an ink composition includes stirring. In some embodiments, the ink composition is stirred until the ink composition is clear without any visible suspended particles.

In some embodiments, a temporary colorant (e.g., any of the temporary colorants described herein) is combined with the ink composition. In some embodiments, the temporary colorant is present in an amount of about 0.1% to about 10% w/w of the ink composition. In some embodiments, the temporary colorant is present in an amount of about 2.5% w/w of the ink composition. In some embodiments, the temporary colorant comprises D&C Black No. 2.

Also provided herein are compositions prepared by the methods described above.

The disclosure will be further described in the following examples, which do not limit the scope of the disclosure described in the claims.

### EXAMPLES

### Example 1. Preparation of Genipin Derivatives

Scheme for the preparation of Compounds 1-13: The following examples are included to describe detailed procedures to synthesize and characterize Compounds 1-13.

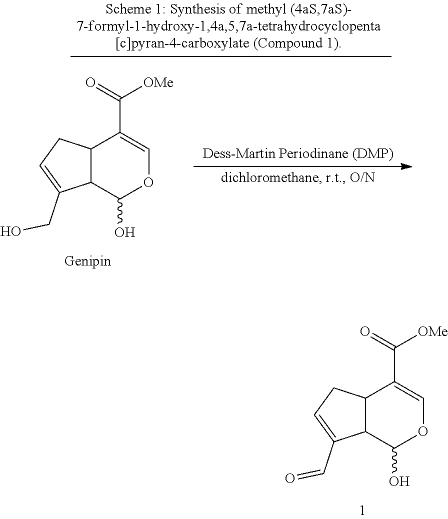 [SMILES: COC(=O)C1=COC(O)C2C(CO)=CCC12 ; COC(=O)C1=COC(O)C2C(C=O)=CCC12]

To a stirred solution of genipin (50 mmol, 1 equivalent) in dichloromethane, was added Dess-Martin periodinane (DMP) (55 mmol, 1.2 equivalent) and the mixture was stirred overnight. A saturated solution of NaHCO₃ and Na₂S₂O₃ was added sequentially to the reaction mixture. The resulting mixture was added to a separatory funnel and extracted with dichloromethane/water (3 times) followed by brine. The combined organic layer was dried with MgSO₄ and concentrated under reduced pressure. The resultant crude material had 10-15% hydration product. The crude mixture was then dissolved in methanol and to this was added a spatula tip of Amberlite® IRA-400 resin (hydroxide form). This combination was stirred at room temperature for 15 minutes. The resin was filtered, the crude material was dry loaded onto silica and purified by flash chromatography (hexane:ethyl acetate 60:40). NMR data was consistent with previous reports (see Tetrahedron, 1993, 49, 10555-10576; Tetrahedron Letters, 1993, 34, 2621-2624).

¹H NMR (400 MHz, CDCl₃) δ 9.80 (s, 1H), 9.69 (s, 1H), 7.59-7.54 (m, 1H), 7.50 (s, 1H), 7.23 (m, 1H), 7.16 (m, 1H), 6.71 (d, J=10.9 Hz, 1H), 5.24 (dd, J=10.9, 2.1 Hz, 1H), 4.79 (dd, J=8.4, 5.9 Hz, 1H), 4.57 (d, J=6.2 Hz, 1H), 3.74 (s, 3H), 3.72 (s, 3H), 3.50 (m, 2H), 3.36-3.18 (m, 2H), 3.02-2.87 (m, 2H), 2.72 (dtd, J=19.8, 2.8, 1.7 Hz, 1H), 2.49-2.33 (m, 1H).

¹³C NMR (100 MHz, CDCl₃) δ 191.8, 191.0, 167.6, 167.4, 161.8, 156.0, 154.7, 153.3, 144.9, 144.6, 109.9, 109.6, 95.6, 95.1, 51.5, 51.4, 48.3, 45.8, 40.8, 39.6, 37.3, 36.0; (M+H)⁺ 225.07637 m/z.

HRMS (DART+): Calculated for C₁₁H₁₃O₅ [M+H]⁺: 225.07575 m/z, found: 225.07637 m/z

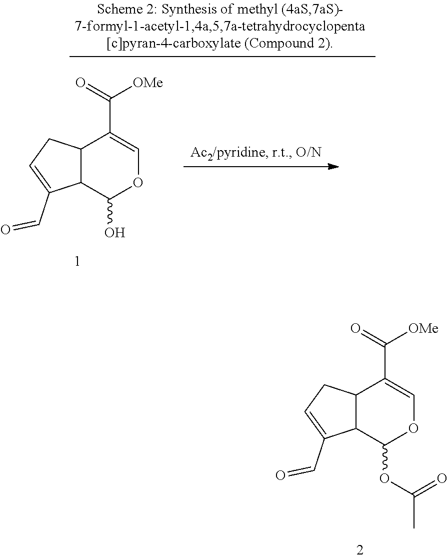 [SMILES: COC(=O)C1=COC(O)C2C(C=O)=CCC12 ; COC(=O)C1=COC(OC(C)=O)C2C(C=O)=CCC12]

A 1:1 (v/v) mixture of acetic anhydride and pyridine were added to Compound 1 and stirred overnight at room temperature. Excess solvent from the mixture was removed under reduced pressure and Compound 2 was isolated by flash column chromatography (hexane:ethyl acetate 80:20).

¹H NMR (400 MHz, CDCl₃) δ 9.76 (s, 1H), 7.46 (d, J=1.1 Hz, 1H), 7.03 (m, 1H), 6.34 (d, J=5.7 Hz, 1H), 3.75 (s, 3H), 3.38 (m, 1H), 3.28 (m, 1H), 3.10 (dddd, J=19.5, 8.0, 2.8, 1.6 Hz, 1H), 2.57 (ddt, J=19.5, 5.6, 2.2 Hz, 1H), 2.11 (s, 3H).

¹³C NMR (100 MHz, CDCl₃): δ 188.7, 169.4, 167.1, 155.8, 152.2, 144.3, 110.6, 90.1, 51.6, 43.6, 39.8, 34.0, 21.0

HRMS (DART+): Calculated for C₁₃H₁₈NO₆ [M+NH4]⁺ 284.11263 m/z, found: 284.11263 m/z.

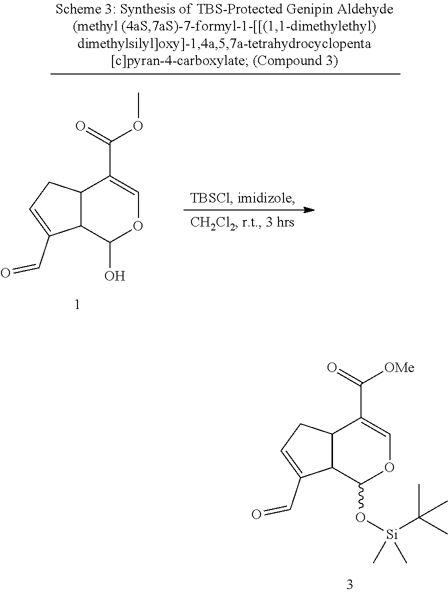 [SMILES: COC(=O)C1=COC(O)C2C(C=O)=CCC12 ; COC(=O)C1=COC(O[Si](C)(C)C(C)(C)C)C2C(C=O)=CCC12]

Tert-butyldimethylsilyl chloride (1.3 equivalents) was added to a stirred solution of Compound 1 (1 equivalent) in anhydrous dichloromethane followed by imidazole (2.5 equivalents). The solution was then stirred for 3 hours at room temperature. The mixture was quenched with water, added to a separatory funnel and extracted with dichloromethane/brine (3λ). The combined organic layer was dried with sodium sulfate and concentrated under reduced pressure. The crude material was dry loaded onto silica and purified by flash chromatography (hexane:diethyl ether 80:20).

¹H NMR (400 MHz, Me₄Si, CDCl₃): δ 9.78 (s, 1H), 7.48 (d, J=0.8 Hz, 1H), 7.00-6.97 (m, 1H), 5.44 (d, J=5.3 Hz, 1H), 3.75 (s, 3H), 3.39-3.31 (m, 1H), 3.21-3.14 (m, 1H), 3.03 (dddd, J=19.8, 8.0, 2.7, 1.6 Hz, 1H), 2.57-2.47 (m, 1H), 0.90 (s, 9H), (s, 6H).

¹³C{¹H} NMR (400 MHz, Me₄Si, CDCl₃): δ 188.8, 167.6, 153.8, 152.8, 145.2, 110.0, 93.6, 51.2, 46.9, 39.6, 34.3, 25.5, 17.9, −4.5, −5.3.

HRMS (DART+) m/z: Calculated for C₁₇H²⁷O₅Si [M+H]⁺: 339.16210 m/z, found: 339.16223 m/z.

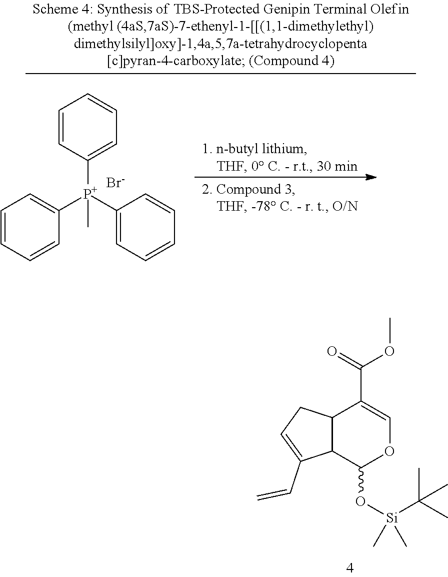 [SMILES: C[P+](c1ccccc1)(c1ccccc1)c1ccccc1 ; [Br-] ; C=CC1=CCC2C(C(=O)OC)=COC(O[Si](C)(C)C(C)(C)C)C12]

To a stirred solution of methyltriphenylphosphonium bromide (1.1 equivalents) in anhydrous tetrahydrofuran at 0° C., n-butyl lithium (1.1 equivalents) was added. The mixture was allowed to warm to room temperature while stirring for 30 minutes, then brought to −78° C. A 0° C. solution of Compound 3 (1 equivalent) in anhydrous tetrahydrofuran was slowly added to the mixture and stirred at −78° C. for 1 hour. The reaction was allowed to gradually warm to room temperature and stirred overnight. The reaction was quenched with saturated ammonium chloride, added to a separatory funnel and extracted with ethyl acetate/brine (3×). The combined organic layer was dried with sodium sulfate and concentrated under reduced pressure. The crude material was dry loaded onto silica and purified by flash chromatography (hexane:ethyl acetate 95:5).

1H NMR (400 MHz, Me4Si, CDCl3): δ 7.57 (s, 1H), 6.46 (dd, J=17.50, 10.89 Hz, 1H), 5.93 (t, J=2.51 Hz, 1H), 5.42 (d, J=17.46 Hz, 1H), 5.13 (d, J=10.85 Hz, 1H), 4.80 (d, J=8.49 Hz, 1H), δ 3.75 (s, 3H), 3.22-3.13 (m, 1H), 2.91 (ddd, J=17.44, 8.78, 3.35 Hz, 1H), 2.78 (t, J=8.68 Hz, 1H), 2.12 (dd, J=17.39, 9.67 Hz, 1H), 0.91 (s, 9H), 0.12 (s, 3H), 0.07 (s, 3H).

¹³C{¹H} NMR (400 MHz, Me₄Si, CDCl₃): δ 168.0, 152.9, 143.4, 132.4, 132.2, 116.6, 110.5, 97.5, 51.2, 46.4, 39.4, 37.2, 25.8, 18.1, −4.6, −4.9.

HRMS (DART+) m/z: [M+H]⁺ calculated for C₁₈H₂₉O₄Si 337.18368; Found 337.18296.

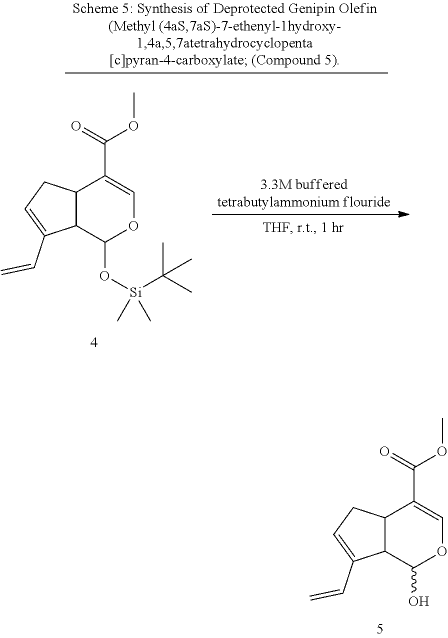 [SMILES: C=CC1=CCC2C(C(=O)OC)=COC(O[Si](C)(C)C(C)(C)C)C12 ; C=CC1=CCC2C(C(=O)OC)=COC(O)C12]

Tetrabutylammonium fluoride hydrate (0.24 g, 0.90 mmol) was dissolved in anhydrous tetrahydrofuran (90 μL). The resulting solution was sonicated for 5 min. Potassium phosphate buffer (0.1 M, 180 μL) was added to the tetrabutylammonium fluoride solution and the entire mixture was sonicated for an additional 5 min yielding a 3.3 M buffered tetrabutylammonium fluoride solution. To a stirred solution of Compound 4 (1 equivalent) in anhydrous tetrahydrofuran, was added 3.3 M buffered tetrabutylammonium fluoride solution (1 equivalent). The reaction was stirred at room temperature for 1.5 hours then the solvent was removed by rotary evaporation. The sample was dry loaded onto silica and purified by flash chromatography (hexane:ethyl acetate 60:40).

¹H NMR (400 MHz, Me₄Si, CDCl₃): δ 7.53 (s, 0.62H), 7.46 (d, J=1.39 Hz, 0.32H), 6.58-6.48 (m, 1H), 6.00 (t, J=2.63 Hz, 0.35H), 5.93 (t, J=2.75 Hz, 0.65H), 5.73 (t, J=3.13 Hz, 0.35H), 5.44 (d, J=17.57 Hz, 0.68H), 5.19-5.10 (m, 1.41H), 4.84 (d, J=8.61 Hz, 0.65H), 3.89 (br, 0.65H), 3.73 (s, 1H), 3.72 (s, 2H), 3.22-3.09 (m, 1.73H), 2.94-2.87 (m, 1H), 2.76 (t, J=8.70 Hz, 0.69H), 2.38 (dd, J=17.11, 9.31 Hz, 0.36H), 2.09 (dd, J=17.45, 9.57 Hz, 0.69H).

¹³C{¹H} NMR (400 MHz, Me₄Si, CDCl₃): δ 168.2, 152.7, 150.7, 142.6, 141.0, 134.9, 132.8, 115.9, 114.0, 110.7, 110.6, 97.0, 91.9, 51.4, 45.4, 43.7, 39.5, 34.4, 36.7, 33.0.

HRMS (DART+) m/z: [M+H]⁺ calculated for C₁₂H₁₅O₄ 223.09631; Found 223.09649.

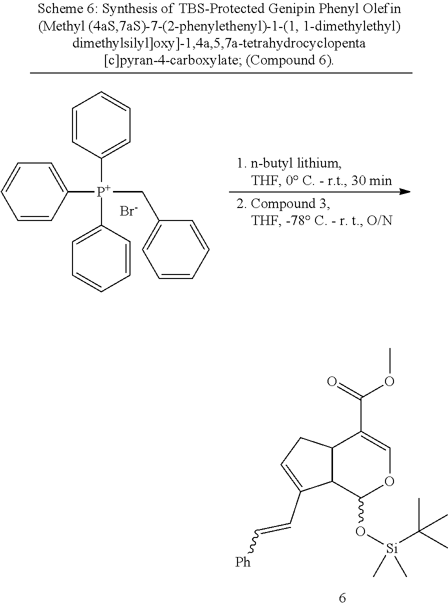 [SMILES: c1ccc(C[P+](c2ccccc2)(c2ccccc2)c2ccccc2)cc1 ; [Br-] ; COC(=O)C1=COC(O[Si](C)(C)C(C)(C)C)C2C(C=Cc3ccccc3)=CCC12]

To a stirred solution of benzyltriphenylphosphonium bromide (1.1 equivalents) in anhydrous tetrahydrofuran at 0° C., was added n-butyl lithium (1.1 equivalents). The mixture was allowed to warm to room temperature while stirring for 30 minutes, then brought to −78° C. A 0° C. solution of Compound 3 (1 equivalent) in anhydrous tetrahydrofuran was slowly added to the mixture and stirred at −78° C. for 1 hour. The reaction was allowed to gradually warm to room temperature and stirred overnight. The reaction was quenched with saturated ammonium chloride, added to a separatory funnel and extracted with ethyl acetate/brine (3×). The combined organic layer was dried with sodium sulfate and concentrated under reduced pressure. The crude material was dry loaded onto silica and purified by flash chromatography (hexane:diethyl ether 90:10). The purified product was isolated as an E/Z isomer mixture.

¹H NMR (400 MHz, Me₄Si, CDCl₃): δ 7.58 (s, 0.4H), 7.45 (s, 0.6H), 7.42-7.37 (m, 0.8H), 7.34-7.27 (m, 3H), 7.23-7.17 (m, 1H), 7.89 (d, J=16.2 Hz, 0.4H), 6.75 (d, J=16.2 Hz, 0.4H), 6.44 (d, J=12.3 Hz, 0.6H), 6.21 (d, J=12.3 Hz, 0.6H), 6.04 (t, J=2.6 Hz, 0.4H), 5.85 (br, 0.6H), 4.84 (d, J=7.0 Hz, 0.6H), 4.81 (d, J=7.6 Hz, 0.4H), 3.74 (s, 1.1H), 3.71 (s, 1.9H), 3.48 (br, 0.6H), 3.27-3.12 (m, 1H), 2.96 (ddd, J=17.5, 8.3, 3.4 Hz, 0.4H), 2.92-2.78 (m, 1H), 2.70 (t, J=7.3 Hz, 0.6H), 2.23-2.07 (m, 1H), 0.88 (s, 5.7H), 0.84 (s, 3.5H), 0.08 (m, 3H), 0.04 (s, 1.9H), −0.03 (s, 1.1H).

¹³C{¹H} NMR (400 MHz, Me₄Si, CDCl₃): δ 168.0, 167.9, 152.9, 152.5, 143.1, 139.3, 138.0, 137.6, 132.6, 132.4, 131.1, 130.2, 128.6, 128.5, 128.2, 127.3, 127.0, 126.4, 126.1, 124.6, 110.7, 110.5, 97.5, 96.3, 51.2, 51.1, 49.9, 46.6, 39.6, 39.5, 37.3, 34.8, 25.6, 18.1, 17.9, −4.4, −4.9, −5.0, −5.2.

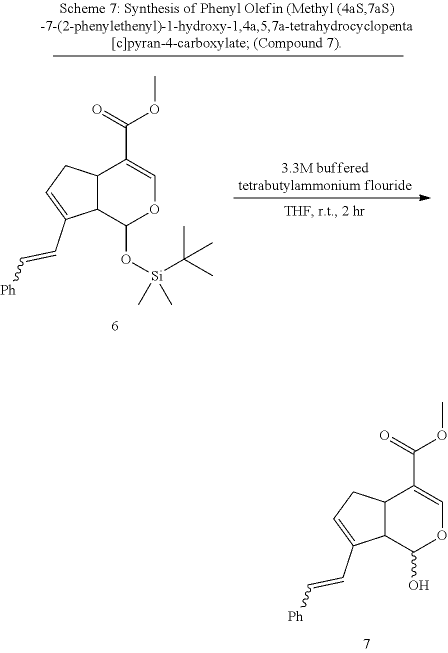 [SMILES: COC(=O)C1=COC(O[Si](C)(C)C(C)(C)C)C2C(C=Cc3ccccc3)=CCC12 ; COC(=O)C1=COC(O)C2C(C=Cc3ccccc3)=CCC12]

Tetrabutylammonium fluoride hydrate (0.24 g, 0.90 mmol) was dissolved in anhydrous tetrahydrofuran (90 μL). The resulting solution was sonicated for 5 min. Potassium phosphate buffer (0.1 M, 180 μL) was added to the tetrabutylammonium fluoride solution and the entire mixture was sonicated for an additional 5 min yielding a 3.3 M buffered tetrabutylammonium fluoride solution. To a stirred solution of Compound 6 (1 equivalent) in anhydrous tetrahydrofuran, was added 3.3 M buffered tetrabutylammonium fluoride solution (1 equivalent). The reaction was stirred at room temperature for 2 hours then the solvent was removed by rotary evaporation. The sample was dry loaded onto silica and purified by flash chromatography (hexane:diethyl ether 60:40). The purified product was isolated as an E/Z isomer mixture.

¹H NMR (400 MHz, Me₄Si, CDCl₃): δ 7.59 (s, 1H), 7.23 (d, J=1.5 Hz, 0.6H), 7.47-7.42 (m, 3.9H), 7.38-7.30 (m, 7.7H), 7.28-7.22 (m, 2.5H), 7.03 (d, J=16.3 Hz, 0.5H), 6.95 (d, J=16.2 Hz, 1H), 6.86 (d, J=16.3 Hz, 1H), 6.58-6.50 (m, 1.5H), 6.38-6.29 (m, 1H), 6.15 (br, 0.5H), 6.07 (br, 1.2H), 5.93 (br, 0.8H), 5.85 (t, J=3.5 Hz, 0.4H), (t, J=3.8 Hz, 0.2H), 4.92 (dd, J=8.7, 5.3 Hz, 1H), 4.79 (t, J=7.9 Hz, 0.9H), 3.92 (d, J=5.3 Hz, 1H), 3.78-3.75 (m, 4.2H), 3.75-3.72 (m, 3H), 3.33-3.04 (m, 4.5H), 3.04-2.85 (m, 4.1H), 2.64 (t, J=7.5 Hz, 0.9H), 2.50 (dd, J=17.1, 9.4 Hz, 0.5H), 2.35-2.25 (m, 0.3H), 2.23-2.05 (m, 2H).

¹³C{¹H} NMR (400 MHz, Me₄Si, CDCl₃): δ 168.0, 167.9, 167.9, 152.6, 152.4, 152.4, 150.6, 150.5, 142.3, 140.6, 139.1, 138.5, 137.8, 137.4, 137.0, 136.5, 136.4, 135.3, 134.5, 133.8, 133.0, 130.8, 130.4, 130.3, 129.8, 129.0, 128.9, 128.8, 128.7, 128.6, 128.6, 128.5, 128.4, 128.1, 127.6, 127.5, 127.4, 127.3, 127.0, 126.7, 126.5, 126.4, 126.3, 126.2, 125.4, 124.6, 124.4, 110.7, 110.7, 110.6, 110.6, 96.9, 96.3, 92.2, 91.9, 51.3, 51.3, 51.2, 48.5, 45.6, 45.6, 44.0, 39.7, 39.5, 39.3, 39.2, 36.7, 35.3, 33.0, 32.6, 30.3, 29.9, 29.7.

HRMS (DART+) m/z: [M+H]⁺ calculated for C₁₈H₁₈O₄ 299.12865; Found 299.12912.

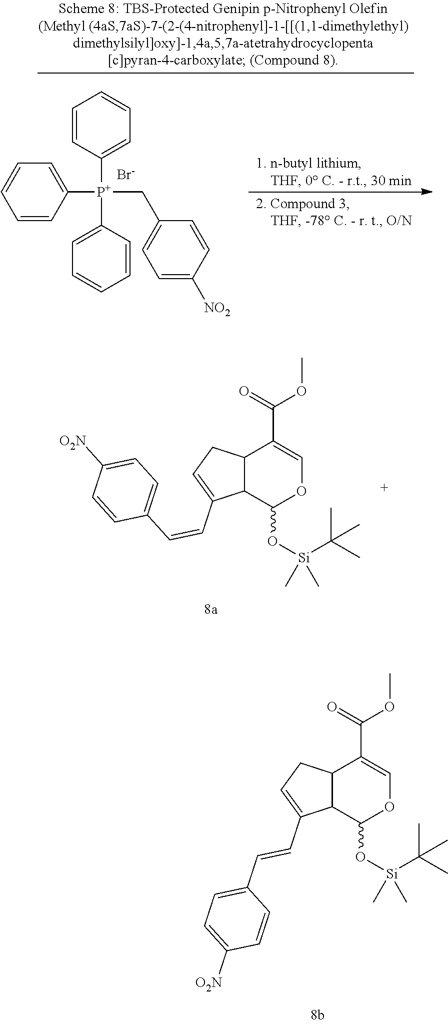 [SMILES: O=[N+]([O-])c1ccc(C[P+](c2ccccc2)(c2ccccc2)c2ccccc2)cc1 ; [Br-] ; COC(=O)C1=COC(O[Si](C)(C)C(C)(C)C)C2C(/C=C\c3ccc([N+](=O)[O-])cc3)=CCC12 ; COC(=O)C1=COC(O[Si](C)(C)C(C)(C)C)C2C(/C=C/c3ccc([N+](=O)[O-])cc3)=CCC12]

To a stirred solution of 4-nitrobenzyltriphenylphosphonium bromide (1.1 equivalents) in anhydrous tetrahydrofuran at 0° C., was added n-butyl lithium (1.1 equivalents). The mixture was allowed to warm to room temperature while stirring for 30 minutes, then brought to −78° C. A 0° C. solution of Compound 3 (1 equivalent) in anhydrous tetrahydrofuran was slowly added to the mixture and stirred at −78° C. for 1 hour. The reaction was allowed to gradually warm to room temperature and stirred overnight. The reaction was quenched with saturated ammonium chloride, added to a separatory funnel and extracted with ethyl acetate/brine (3×). The combined organic layer was dried with sodium sulfate and concentrated under reduced pressure. The crude material was dry loaded onto silica and purified by flash chromatography (hexane:diethyl ether 90:10). E and Z isomers were separated successfully.

¹H NMR (400 MHz, Me₄Si, CDCl₃) of Z isomer: δ 8.16 (d, J=8.7 Hz, 2H), 7.53-7.47 (m, 3H), 6.47 (d, J=12.4 Hz, 1H), 6.40 (d, J=12.7 Hz, 1H), 5.90 (br, 1H), 4.85 (d, J=7.4 Hz, 1H), 3.74 (s, 3H), 3.20 (q, J=8.1 Hz, 1H), 2.88 (dd, J=17.7, 8.4 Hz, 1H), 2.63 (t, J=7.4 Hz, 1H), 2.14 (ddd, J=17.7, 8.2, 1.8 Hz, 1H), 0.90 (s, 9H), 0.12 (s, 3H), 0.09 (s, 3H).

¹³C{¹H} NMR (400 MHz, Me₄Si, CDCl₃) of Z isomer: δ 167.8, 152.5, 146.5, 144.9, 138.7, 134.2, 129.7, 129.3, 128.0, 123.6, 110.6, 96.5, 51.2, 50.0, 39.7, 35.1, 17.9, −4.3, −5.2.

¹H NMR (400 MHz, Me₄Si, CDCl₃) of E isomer: δ 8.16 (d, J=8.8 Hz, 2H), 7.58 (s, 1H), 7.50 (d, J=8.8 Hz, 2H), 7.06 (d, J=16.1 Hz, 1H), 6.80 (d, J=16.2 Hz, 1H), 6.20 (t, J=2.7 Hz, 1H), 4.80 (d, J=8.6 Hz, 1H), 3.74 (s, 3H), 3.23 (q, J=8.6 Hz, 1H), 3.00 (ddd, J=18.1, 8.4, 3.5 Hz, 1H), 2.89 (t, J=8.0 Hz, 1H), 2.20 (dd, J=18.0, 9.9 Hz, 1H), 0.82 (s, 9H), 0.08 (s, 3H), 0.05 (s, 3H).

¹³C{¹H} NMR (400 MHz, Me₄Si, CDCl3) of E isomer: δ 167.8, 152.9, 146.6, 144.2, 142.7, 136.6, 129.0, 128.9, 126.6, 124.1, 110.3, 97.3, 51.3, 46.5, 39.8, 37.3, 18.0, −4.8, −4.9.

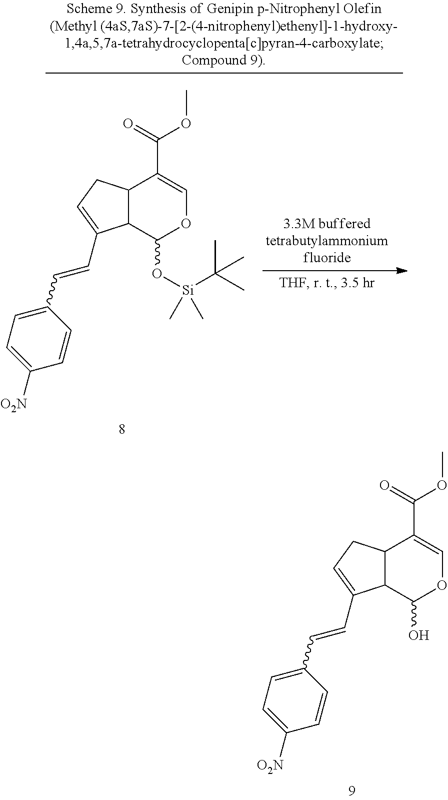 [SMILES: COC(=O)C1=COC(O[Si](C)(C)C(C)(C)C)C2C(C=Cc3ccc([N+](=O)[O-])cc3)=CCC12 ; COC(=O)C1=COC(O)C2C(C=Cc3ccc([N+](=O)[O-])cc3)=CCC12]

Tetrabutylammonium fluoride hydrate (0.24 g, 0.90 mmol) was dissolved in anhydrous tetrahydrofuran (90 μL). The resulting solution was sonicated for 5 min. Potassium phosphate buffer (0.1 M, 180 μL) was added to the tetrabutylammonium fluoride solution and the entire mixture was sonicated for an additional 5 min yielding a 3.3 M buffered tetrabutylammonium fluoride solution. To a stirred solution of Compound 8 (1 equivalent) in anhydrous tetrahydrofuran, was added 3.3 M buffered tetrabutylammonium fluoride solution (1 equivalent). The reaction was stirred at room temperature for 3.5 hours then the solvent was removed by rotary evaporation. The sample was dry loaded onto silica and purified by flash chromatography (hexane:diethyl ether 50:50).

¹H NMR (400 MHz, Me₄Si, CDCl₃): δ 8.24-8.16 (m, 2H), 7.61-7.51 (m, 3H), 7.18 (d, J=17.0 Hz, 0.3H), 7.10 (d, J=16.4 Hz, 0.7H), 6.90 (d, J=16.2 Hz, 0.7H), 6.61-6.53 (m, 0.3H), 6.33 (t, J=2.4 Hz, 0.2H), 6.25 (t, J=2.7 Hz, 0.7H), 5.86 (d, J=2.5 Hz, 4.93 (d, J=8.8 Hz, 0.7H), 3.81-3.76 (m, 3H), 3.63 (br, 0.7H), 3.30 (m, 1H), 3.21 (q, J=8.9 Hz, 0.3H), 3.17-3.10 (m, 0.1H), 3.10-2.99 (m, 1H), 2.96-2.85 (m, 1H), 2.53 (dd, J=17.8, 9.8 Hz, 0.3H), 2.22 (dd, J=18.0, 9.6 Hz, 0.8H).

¹³C{¹H} NMR (400 MHz, Me₄Si, CDCl₃): δ 167.8, 167.7, 152.3, 152.2, 150.4, 146.7, 146.6, 144.2, 143.7, 142.1, 140.2, 139.3, 136.8, 129.4, 129.0, 128.9, 128.7, 128.4, 126.7, 126.7, 126.2, 124.2, 124.1, 123.7, 110.7, 110.6, 96.7, 96.0, 91.7, 51.4, 51.4, 49.0, 45.4, 43.9, 39.9, 39.8, 36.8, 35.1, 34.2, 33.2, 30.3, 29.7.

HRMS (DART+) m/z: [M+H]⁺ calculated for C₁₈H₁₈NO₆ 344.11316; Found 344.11286.

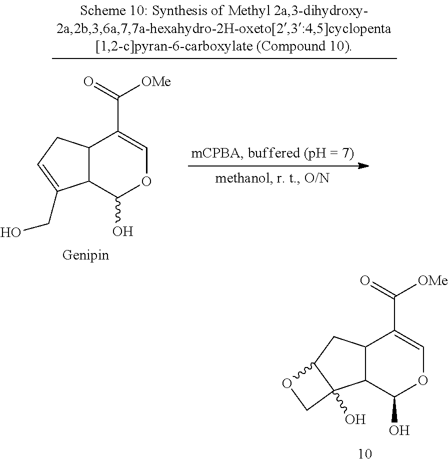 [SMILES: COC(=O)C1=COC(O)C2C(CO)=CCC12 ; COC(=O)C1=CO[C@@H](O)C2C1CC1OCC12O]

Genipin (5 mmol, 1 equivalent) was dissolved in 20 ml of methanol. A few drops of phosphate buffer (pH=7) and mCPBA (6 mmol, 1.2 equivalent) was added to the reaction mixture and the mixture was stirred at room temperature overnight. The solvent was removed under reduced pressure and the residue was dissolved in 5% (w/w) K₂CO₃. The product was extracted with ethyl acetate and dried over MgSO₄. The product was purified using silica gel column chromatography (dichloromethane:ethyl acetate) yielding white needles.

¹H NMR (400 MHz, DMSO-d₆): δ 7.28 (d, J=0.8 Hz, 1H, H-3), 5.98 (d, J=4.2 Hz, 1H, OH), 5.51 (s, 1H, OH), 4.88 (d, J=4.1 Hz, 1H, H-1), 4.27 (ddt, J=2.8, 1.9, 1.0 Hz, 1H, H-7), 4.03 (d, J=9.0 Hz, 1H, H-10a), 3.69 (d, J=9.0 Hz, 1H, H-10b), 3.61 (s, 3H, OCH3), 3.11-3.04 (m, 1H), 2.37 (d, J=6.6 Hz, 1H), 2.26 (ddd, J=12.1, 4.4, 2.7 Hz, 1H), 1.75 (d, J=12.1 Hz, 1H).

¹³C NMR (101 MHz, DMSO-d₆): δ 166.95, 154.38, 110.20, 98.65, 90.70, 84.24, 70.48, 67.75, 51.41, 33.13, 32.02.

HRMS (DART+) m/z: 265.0865 (M+Na)+

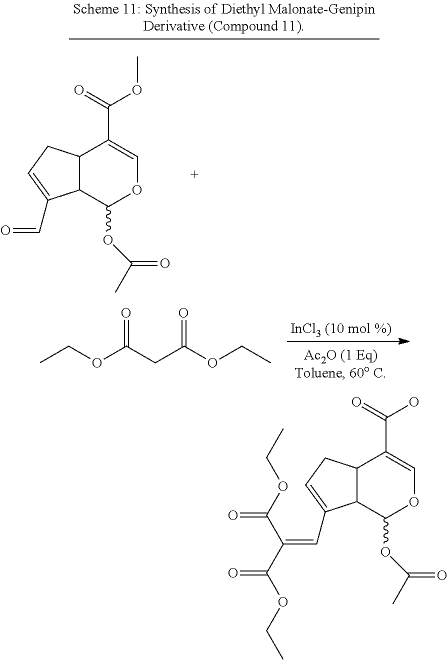

Acetylated genipin aldehyde (Compound 2) (1 equivalent) was added to a solution of InCl₃ (1.1 equivalents) in toluene (0.5 M). Diethyl malonate (1.1 equivalent) and acetic anhydride (1 equivalent) were added. The reaction mixture was stirred at 60° C. for 18 hours. The crude reaction mixture was quenched with saturated sodium bicarbonate and extracted with ethyl acetate. The resulting compound was isolated from the crude reaction mixture using flash column chromatography.

¹H NMR (400 MHz, CDCl₃): δ 7.48 (s, 1H), 7.31 (s, 1H), 5.65 (d, J=8.6 Hz, 1H), 4.39-4.19 (m, 4H), 3.74 (s, 3H), 3.32 (m, 1H), 3.10-2.94 (m, 2H), 2.34-2.24 (m, 1H), 2.12 (s, 3H), 1.30 (t, J=7.1, 6H).

¹³C NMR (100 MHz, CDCl₃): δ 169.3, 167.2, 165.9, 164.3, 152.2, 142.8, 137.6, 136.7, 126.6, 111.0, 92.4, 61.7, 61.7, 51.6, 45.4, 40.1, 35.6, 20.9, 14.2, 14.1.

HRMS (ESI+): Calculated for C₂₀H₂₄O₉Na [M+Na]⁺: 431.1313 m/z, found: 431.1303 m/z.

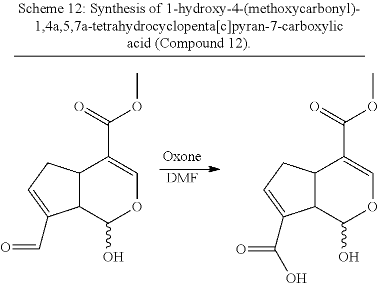

Genipin aldehyde (Compound 1) was added to a suspension of oxone (potassium peroxymonosulfate; 4 equivalents) in DMF (0.2 M), and the reaction mixture was stirred at room temperature for four days. The crude mixture was added to a separatory funnel with ethyl acetate and brine. The aqueous layer was washed thoroughly with ethyl acetate. The combined ethyl acetate layers was subsequently extracted (3×) with saturated NHCO₃. The combined aqueous basic layer was carefully re-acidified with concentrated HCl until the pH was below 7. The acidified solution was then extracted with ethyl acetate (3×). The combined ethyl acetate layer was dried with Na₂SO₄ and concentrated.

¹H NMR (400 MHz, CDCl₃): δ 7.57 (s, 1H), 7.50 (s, 0.4H), 7.23 (s, 0.4H), 7.20 (t, J=2.7 Hz, 1H), 5.53-5.45 (m, 0.4H), 4.85 (d, J=8.4 Hz, 1H), 3.74 (s, 5H), 3.41-3.26 (m, 2H), 3.15 (ddd, J=19.0, 8.5, 3.2 Hz, 1H), 3.02-2.92 (m, 0.4H), 2.89 (m, 1H), 2.6-2.52 (m, 0.4H), 2.34 (ddt, J=19.0, 9.4, 2.2 Hz, 1H).

¹³C NMR (100 MHz, CDCl₃): δ 170.3, 170.2, 167.8, 167.7, 153.3, 153.1, 153.0, 150.8, 134.0, 133.0, 110.0, 95.9, 93.9, 51.6, 51.5, 46.9, 46.3, 40.5, 39.7, 36.4, 35.1.

HRMS (DART+): Calculated for C₁₁H₁₃O₆ [M+H]⁺: 241.07066 m/z, found: 241.07077 m/z.

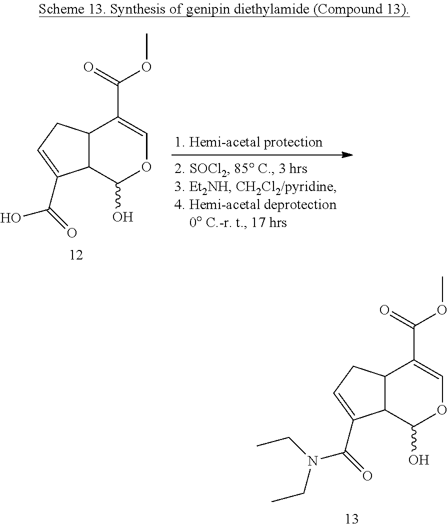 [SMILES: COC(=O)C1=COC(O)C2C(C(=O)O)=CCC12 ; CCN(CC)C(=O)C1=CCC2C(C(=O)OC)=COC(O)C12]

Genipin carboxylic acid (Compound 12, which had been protected at the hemi-acetal), was dissolved in SOCl₂ (1.1 M) and set to 85° C. for 3 hrs. After completion, the excess SOCl₂ was removed under reduced pressure. The crude residue was diluted with dichloromethane/pyridine (1:1 v/v) and cooled to 0° C. Diethyl amine was added dropwise, and the reaction was allowed to warm to room temperature and left for 17 hours. The crude residue was separated between dichloromethane and water. The aqueous layer was washed with DCM 3×. The combined dichloromethane layer was dried with Na₂SO₄ and concentrated under reduced pressure and purified using flash column chromatography. Compound 13 was isolated by acid deprotection followed by flash column chromatography.

¹H NMR (400 MHz, CDCl₃): δ 8.38 (s, 1H), 7.60 (s, 1H), 6.29 (m, 1H), 4.92 (d, J=8.3 Hz, 1H), 3.74 (s, 3H), 3.62 (m, 1H), 3.56-3.46 (m, 1H), 3.40 (m, 2H), 3.25-3.16 (m, 1H), 3.08 (ddd, J=17.7, 8.4, 3.2 Hz, 1H), 1.22 (m, 6H).

¹³C NMR (100 MHz, CDCl₃): δ 169.0, 167.9, 153.6, 138.1, 135.6, 109.6, 96.5, 51.4, 49.8, 44.2, 40.9, 40.4, 36.1, 14.9, 12.7.

HRMS (DART+): Calculated for C₁₅H₂₂NO₅ [M+H]⁺: 296.14925 m/z, found: 296.14962 m/z.

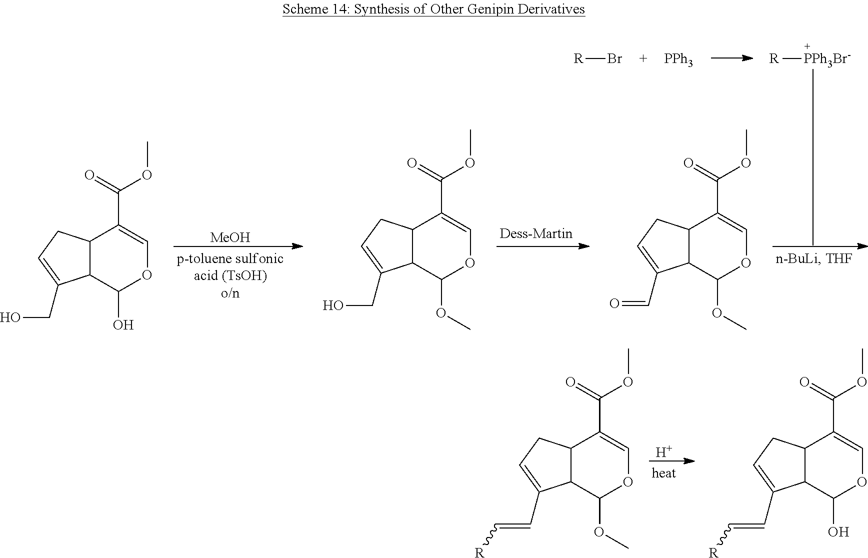

To a stirred solution of genipin (1 equivalents) in methanol, p-toluene sulfonic acid (TsOH) is added and the reaction mixture is incubated.

To a stirred solution of methyl 7-(hydroxymethyl)-1-methoxy-1,4a,5,7a-tetrahydrocyclopenta[c]pyran-4-carboxylate in solvent, Dess-Martin periodinane (1 equivalents) is added and the reaction mixture is stirred. The reaction mixture is extracted.

To a solution of R—Br in THF n-BuLi (X mL, X mol) is added. Next, triphenylphosphine (PPh₃) is added and the reaction mixture is stirred. Methyl 7-formyl-1-m ethoxy-1,4a, 5,7a-tetrahydrocyclopenta[c]pyran-4-carboxylate is added to the reaction mixture and allowed to stir. The product is isolated. In some embodiments, R is A-CH₂, where A is H or optionally substituted C₆₋₁₀ aryl.

Alternatively, a solution of R—Br in toluene is added to triphenylphosphine (1 equivalent) and brought to reflux. In some embodiments, R is A-CH₂, where A is H or optionally substituted C₆₋₁₀ aryl. The product is isolated by precipitation and filtration. In a THF solution, n-BuLi (2 equivalents) is added to the salt. Next, methyl 7-formyl-1-methoxy-1,4a,5,7a-tetrahydrocyclopenta[c]pyran-4-carboxylate (1 equivalent) is added to the reaction mixture and allowed to stir. The product is isolated.

A solution of the product above in acid is stirred. The resulting compound is isolated.

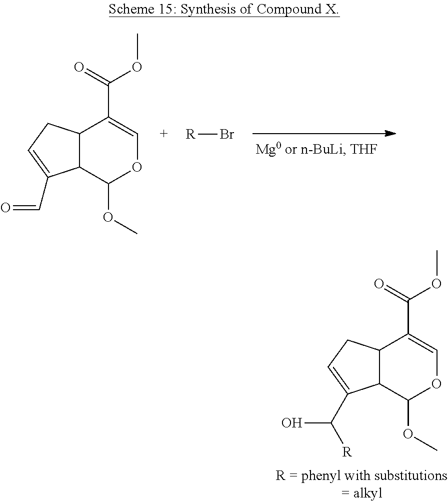

To a solution of R—Br in THF is added n-BuLi or Mg⁰ and allowed to stir. Next, methyl 7-formyl-1-methoxy-1,4a,5,7a-tetrahydrocyclopenta[c]pyran-4-carboxylate is added to the reaction mixture. The resulting compound is isolated. A solution of the product in acid is stirred. The final compound is isolated.

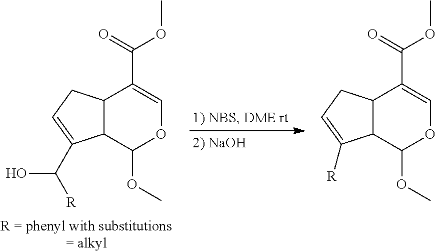

The compound is added to a solution of dimethoxyethane (DME). N-bromosuccinimide (NBS) is added and the reaction mixture is stirred and incubated. NaOH is added and the reaction mixture is stirred. The resulting compound is isolated. A solution of the product in acid is stirred. The final compound is isolated.

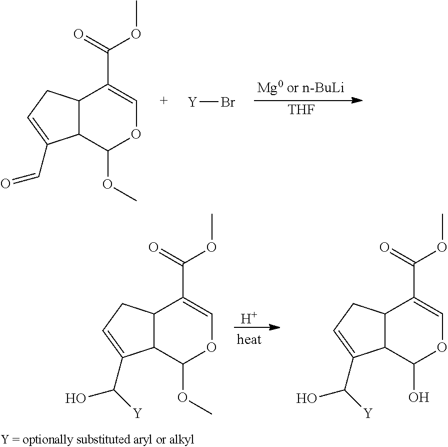

In some embodiments, to a solution of Y—Br in THF is added n-BuLi or Mg⁰ and allowed to stir. Next, methyl 7-formyl-1-methoxy-1,4a,5,7a-tetrahydrocyclopenta[c]pyran-4-carboxylate is added to the reaction mixture. The resulting compound is isolated. A solution of the product in acid is stirred. The final compound is isolated.

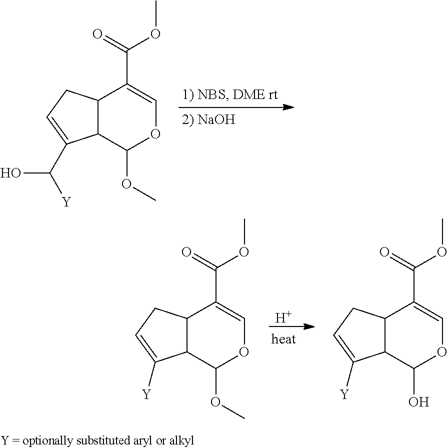

The compound is added to a solution of dimethoxyethane (DME). N-bromosuccinimide (NBS) is added and the reaction mixture is stirred and incubated. NaOH is added and the reaction mixture is stirred. The resulting compound is isolated. A solution of the product in acid is stirred. The final compound is isolated.

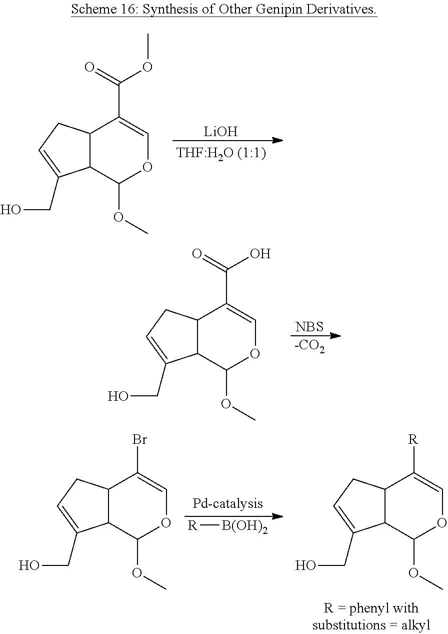

To a stirred solution of genipin in THF:H₂O (1:1) LiOH is added, and the reaction mixture is incubated. The product is isolated.

To a solution of said product is added NBS, and the reaction mixture is incubated. The product is isolated.

To a solution of said product is added a Pd-catalyst and R—B(OH)₂, and the reaction mixture is incubated. The resulting compound is isolated.

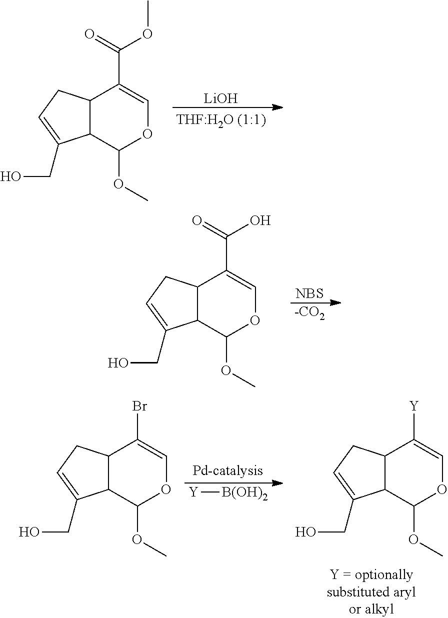

In some embodiments, to a stirred solution of genipin in THF:H₂O (1:1) LiOH is added, and the reaction mixture is incubated. The product is isolated.

To a solution of said product is added NBS, and the reaction mixture is incubated. The product is isolated.

To a solution of said product is added a Pd-catalyst and Y—B(OH)₂, and the reaction mixture is incubated. The resulting compound is isolated.

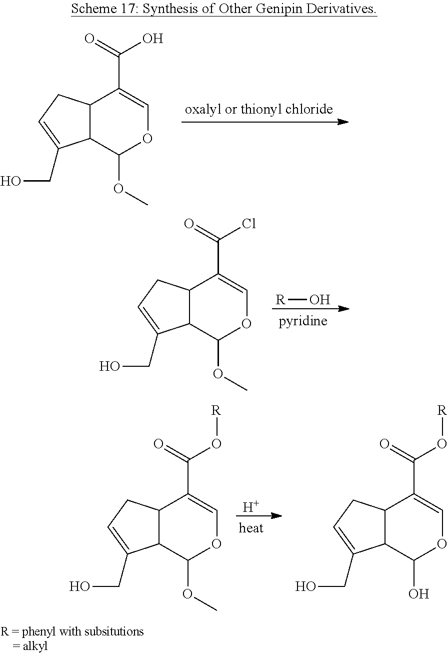

To a stirred solution of genipin is added oxyalyl or thionyl chloride, and the reaction mixture is incubated. The product is isolated.

To a solution of said product is added R—OH and pyridine, and the reaction mixture is incubated. The product is isolated.

To a solution of said product is added acid, and the reaction mixture is incubated. The resulting compound is isolated.

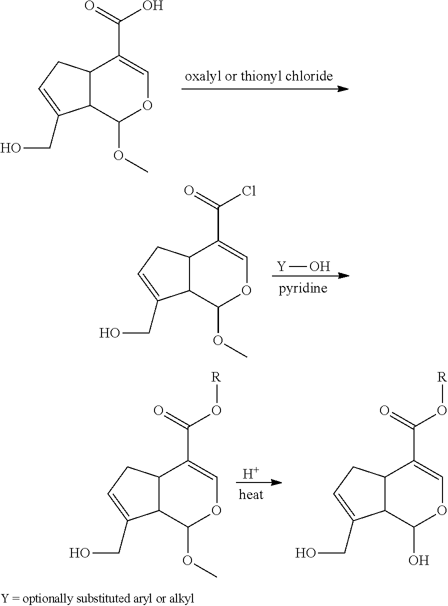

In some embodiments, to a stirred solution of genipin is added oxyalyl or thionyl chloride, and the reaction mixture is incubated. The product is isolated.

To a solution of said product is added Y—OH and pyridine, and the reaction mixture is incubated. The product is isolated.

To a solution of said product is added acid, and the reaction mixture is incubated. The resulting compound is isolated.

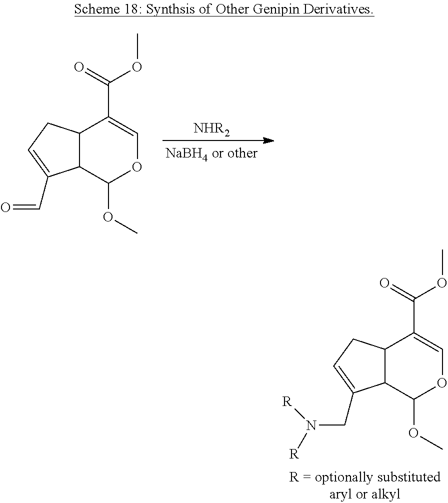

To a solution of genipin is added NHR₂ and a reducing agent (e.g., NaBH₄), and the reaction mixture is incubated. The resulting compound is isolated.

To a solution of said product is added acid, and the reaction mixture is incubated. The resulting compound is isolated.

### Example 2. Preparation of Lawsone Derivatives

Scheme 14: Synthesis of Compound 14: 2-Hydroxy-3-phenyl-1,4-Naphthalenedione.

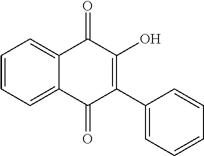 [SMILES: O=C1C(O)=C(c2ccccc2)C(=O)c2ccccc21]

The above compound was made using established protocols (see, e.g., Synlett, 2006, 16, 2597-2600 and New J. Chem. 2016, 40, 7643-7656). The product was isolated via precipitation or using a gradient column of hexane:ethyl acetate with 0.5% acetic acid.

¹H NMR (400 MHz, CDCl₃): δ 8.26-8.11 (m, 2H), 7.86-7.70 (m, 2H), 7.56-7.37 (m, 5H).

¹³C NMR (100 MHz, CDCl₃): δ 183.9, 182.0, 152.3, 135.5, 133.3, 133.0, 130.8, 130.1, 129.4, 128.8, 128.1, 127.5, 126.3, 122.3;

HRAM (ESI+): Calculated for C₁₆H₁₁O₃ [M+H]⁺: 251.0703 m/z, found: 251.0703 m/z.

### Example 3. Reaction of Genipin and Lawsone Derivatives with Amino Acids

Reaction of Compound 1 with Amino Acids to Mimic Skin Binding

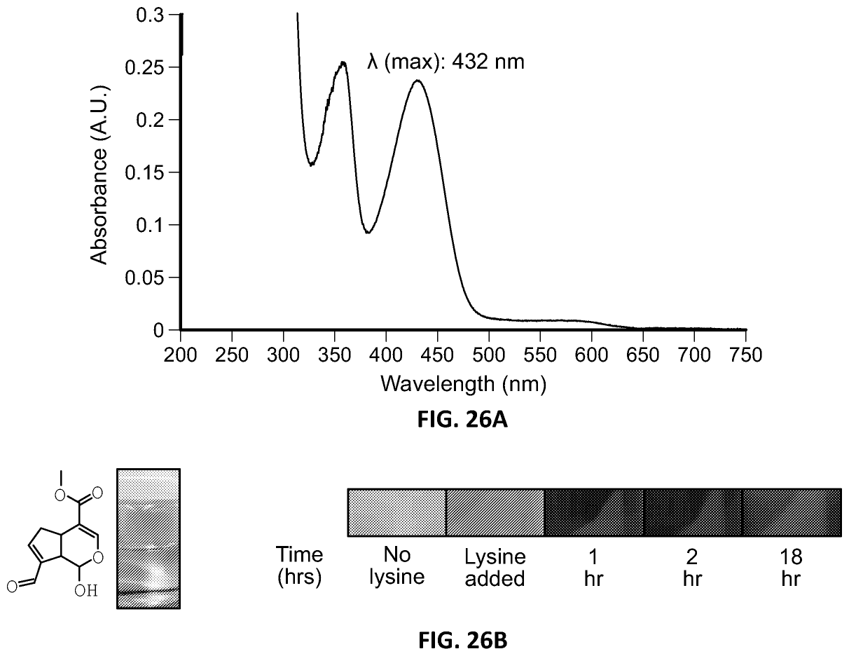

Compound 1 was weighed out (0.03 mmol) into a small vial equipped with a magnetic stir bar. To this vial, 1 mL of methanol was added and stirred until solubilized. L-lysine was then added to the stirred solution (2 equivalents) and the reaction mixture was capped and stirred over 44 hours at room temperature. A needle was punctured through the vial cap to ensure the reaction is conducted under open air. This method produced a yellow dye with a λₘₐₓ=436 nm. See FIGS. 1A and 1B. See also FIGS. 26A and 26B.

Reaction of Compound 10 with Amino Acids to Mimic Skin Binding.

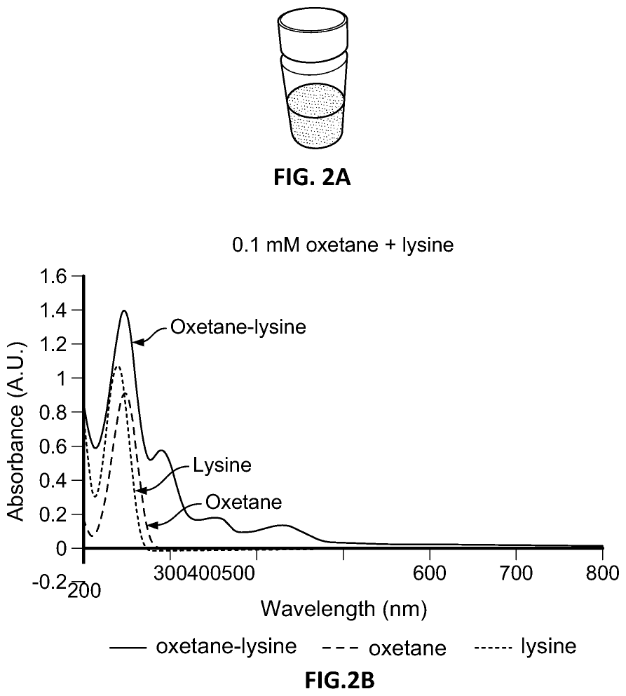

Compound 10 was weighed out (n=0.03 mmol) into a small vial equipped with a magnetic stir bar. To this, 1 mL of methanol was added and stirred until solubilized. L-lysine was then added to the stirred solution (2 equivalents) and the reaction mixture was capped and stirred overnight at room temperature. A needle was punctured through the vial cap to ensure the reaction is conducted under open air. This produced a yellow dye with a λₘₐₓ=433 nm. See FIGS. 2A and 2B.

Reaction of Compound 14 with Amino Acids to Mimic Skin Binding.

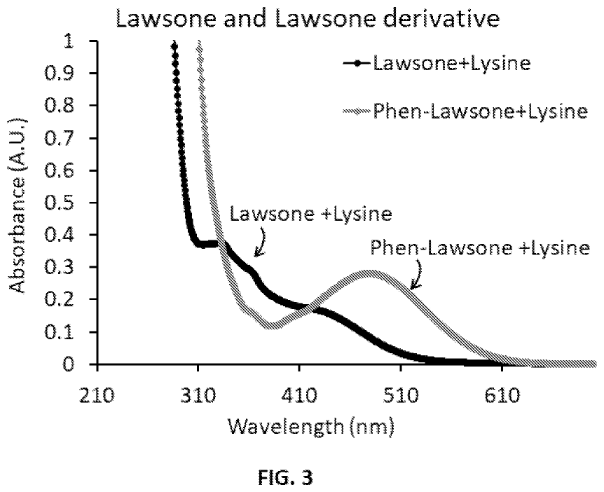

Compound 14 was weighed out (n=0.03 mmol) into a small vial equipped with a magnetic stir bar. To this, 1 mL of methanol was added and stirred until solubilized. L-lysine was then added to the stirred solution (2 equivalents) and the reaction mixture was capped and stirred overnight at room temperature. A needle was punctured through the vial cap to ensure the reaction is conducted under open air. This produces a red dye with a λₘₐₓ=480 nm. See FIG. 3.

Reaction of Compound 11 with Amino Acids to Mimic Skin Binding.

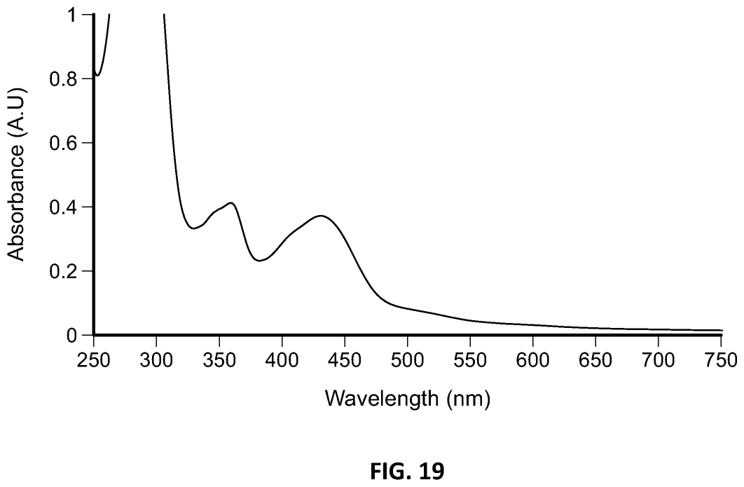

Compound 11 was weighed out (n=0.03 mmol) into a small vial equipped with a magnetic stir bar. To this, 2.5 mL of methanol was added followed by L-lysine (0.06 mmol, 2 equivalents). The reaction mixture was stirred for 24 hours at room temperature under open air. Compound 11 and Compound 11+lysine were diluted with methanol to make 0.06 mM solutions for UV-VIS characterization. See FIG. 19.

Reaction of Compound 13 with Amino Acids to Mimic Skin Binding.

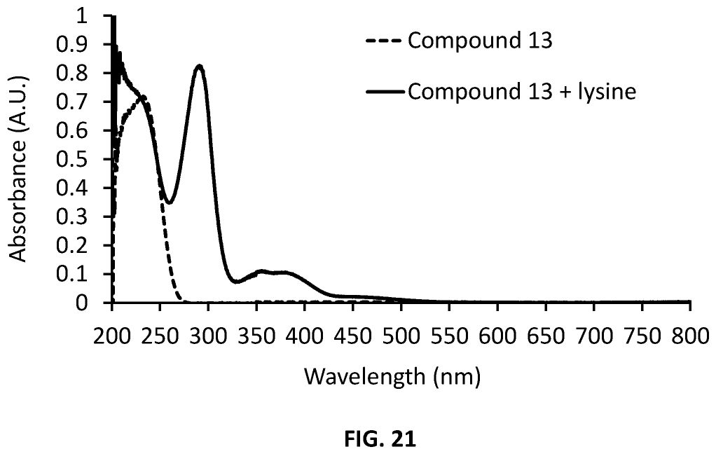

Compound 13 was weighed out (n=0.03 mmol) into a small vial equipped with a magnetic stir bar. To this, 1.5 mL of methanol was added followed by L-lysine (0.06 mmol, 2 equivalents). The reaction mixture was stirred for 24 hours at room temperature under open air. Compound 13+lysine were diluted with methanol to make 0.06 mM solutions for UV-VIS characterization. See FIG. 21.

Reaction of Compound 5 with Amino Acids to Mimic Skin Binding.

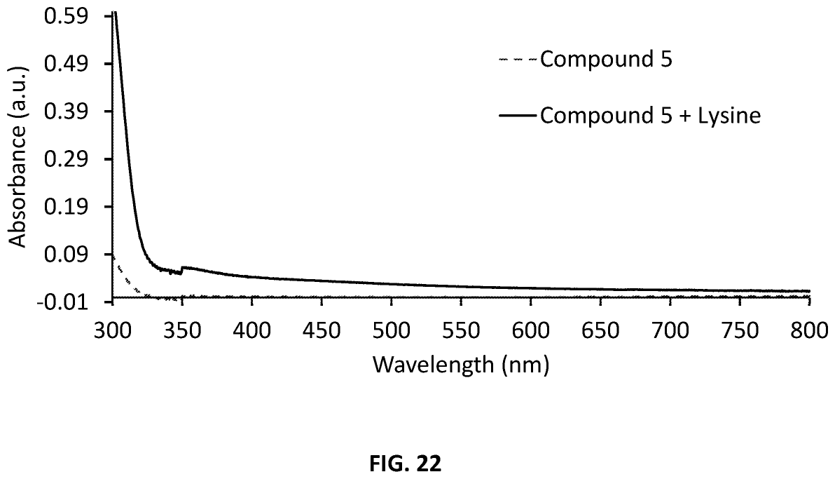

Compound 5 was weighed out (n=0.05 mmol) into a small vial equipped with a magnetic stir bar. To this, 2.5 mL of methanol was added followed by L-lysine (0.1 mmol, 2 equivalents). The reaction mixture was stirred for 24 hours at room temperature under open air. Compound 5 and Compound 5+lysine were diluted with methanol to make 0.1 mM solutions for UV-VIS characterization. See FIG. 22.

Reaction of Compound 7 with Amino Acids to Mimic Skin Binding.

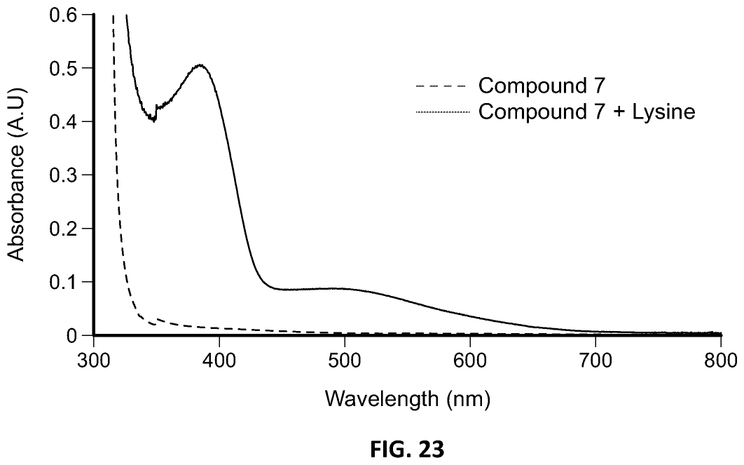

Compound 7 was weighed out (n=0.04 mmol) into a small vial equipped with a magnetic stir bar. To this, 2 mL of methanol was added followed by L-lysine (0.08 mmol, 2 equivalents). The reaction mixture was stirred for 24 hours at room temperature under open air. Compound 7 and Compound 7+lysine were diluted with methanol to make 0.1 and 0.15 mM solutions, respectively, for UV-VIS characterization. See FIG. 23.

Reaction of Compound 9 with Amino Acids to Mimic Skin Binding.

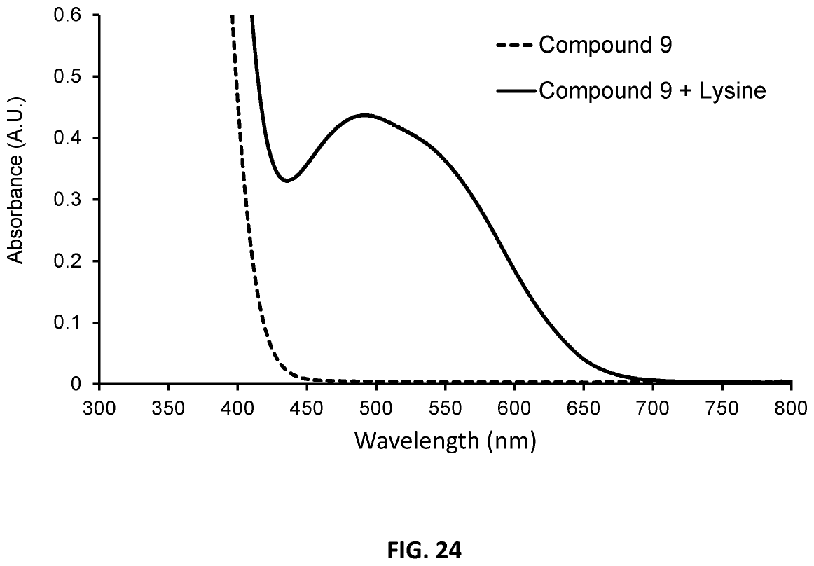

Compound 9 was weighed out (n=0.03 mmol) into a small vial equipped with a magnetic stir bar. To this, 1.5 mL of methanol was added followed by L-lysine (0.06 mmol, 2 equivalents). The reaction mixture was stirred for 24 hours at room temperature under open air. Compound 9 and Compound 9+lysine were diluted with methanol to make 0.1 and 0.15 mM solutions, respectively, for UV-VIS characterization. See FIG. 24.

Reaction of Genipin with Amino Acids to Mimic Skin Binding.

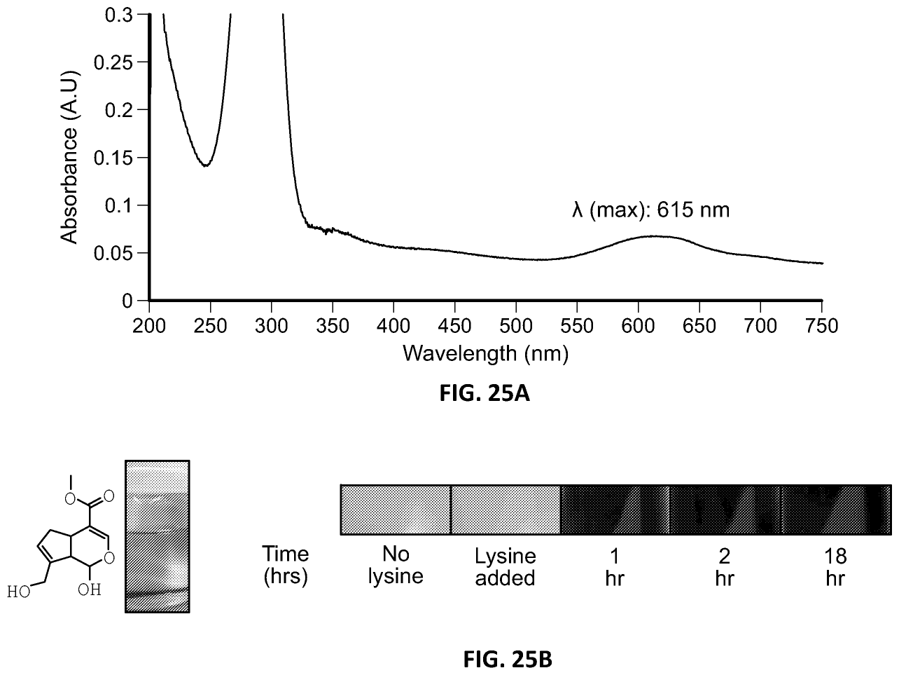

Genipin was weighed out (n=0.03 mmol) into a small vial equipped with a magnetic stir bar. To this, 1.5 mL of methanol was added followed by L-lysine (0.06 mmol, 2 equivalents). The reaction mixture was stirred for 24 hours at room temperature under open air. Genipin+lysine were diluted with methanol to make 0.06 mM solutions for UV-VIS characterization. See FIGS. 25A and 25B.

Reaction of Compound 12 with Amino Acids to Mimic Skin Binding.

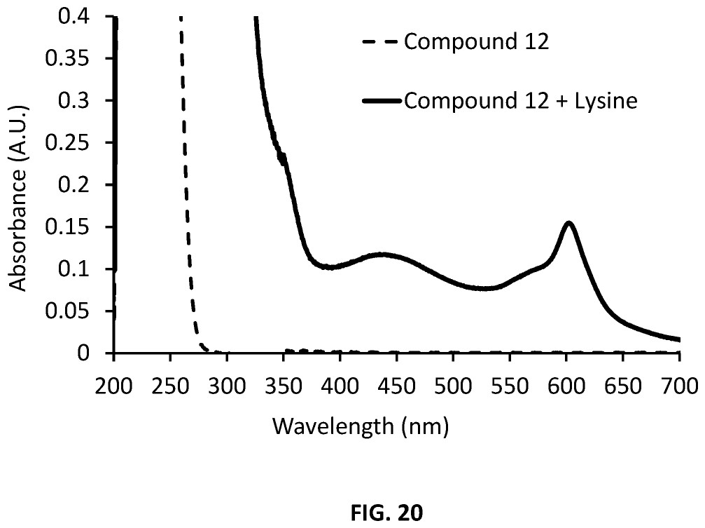

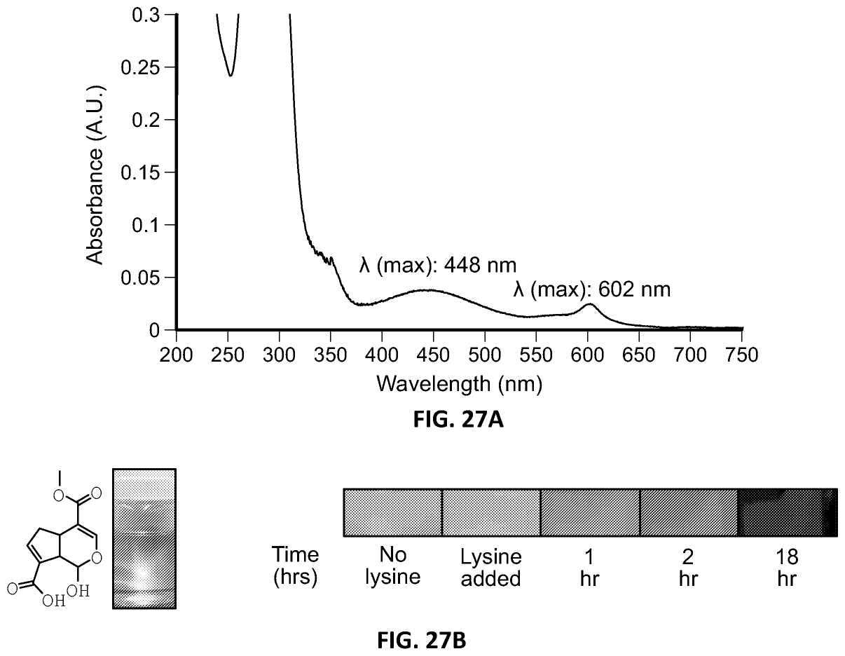

Compound 12 was weighed out (n=0.03 mmol) into a small vial equipped with a magnetic stir bar. To this, 1.5 mL of methanol was added followed by L-lysine (0.06 mmol, 2 equivalents). The reaction mixture was stirred for 24 hours at room temperature under open air. Compound 12+lysine were diluted with methanol to make 0.06 mM solutions for UV-VIS characterization. See FIG. 20. See also FIGS. 27A and 27B.

### Example 4. Combinations of Genipin, Genipin Derivatives, Lawsone, and Lawsone Derivatives

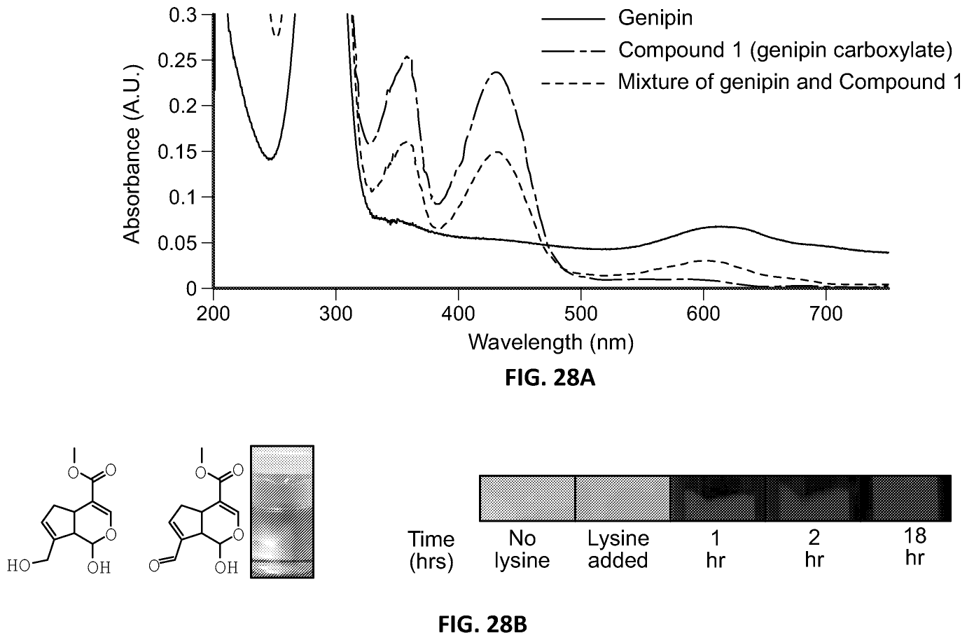

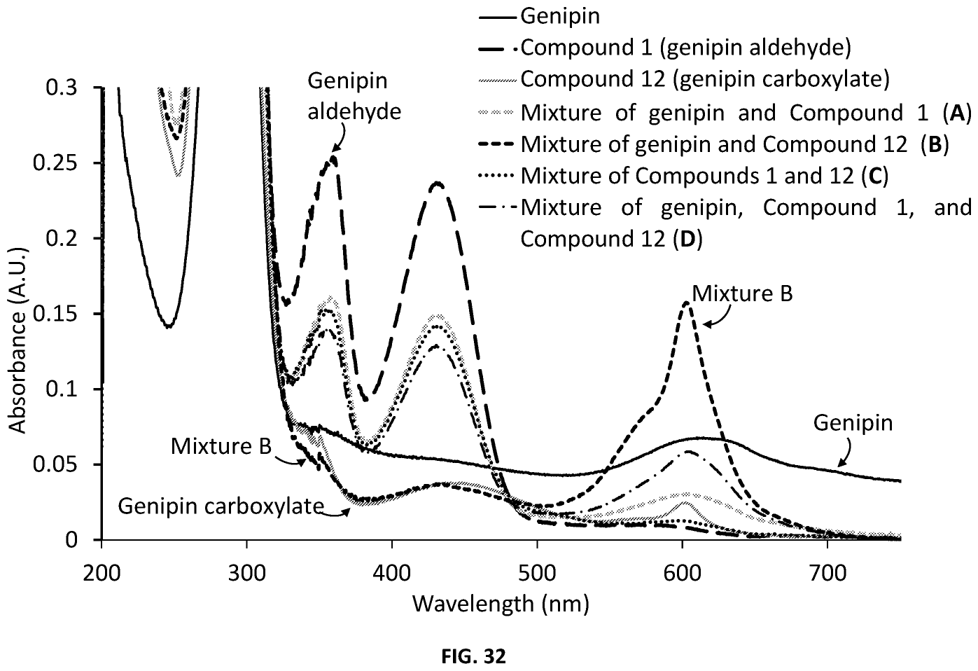

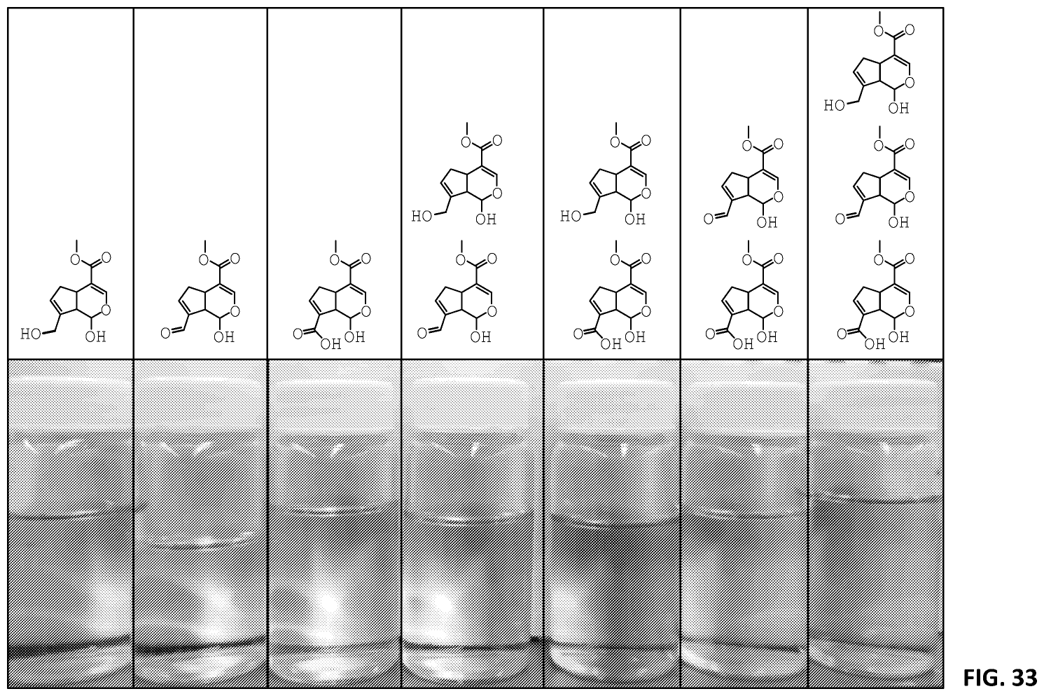

Genipin and Compound 1 were weighed out (n=0.03 mmol each) into a small vial equipped with a magnetic stir bar. To this, 1.5 mL of methanol was added followed by L-lysine (0.12 mmol, 2 equivalents). The reaction mixture was stirred for 24 hours at room temperature under open air. This mixture was diluted with methanol to make 0.06 mM solutions for UV-VIS characterization. See FIGS. 28A and 28B. See also FIGS. 32 and 33.

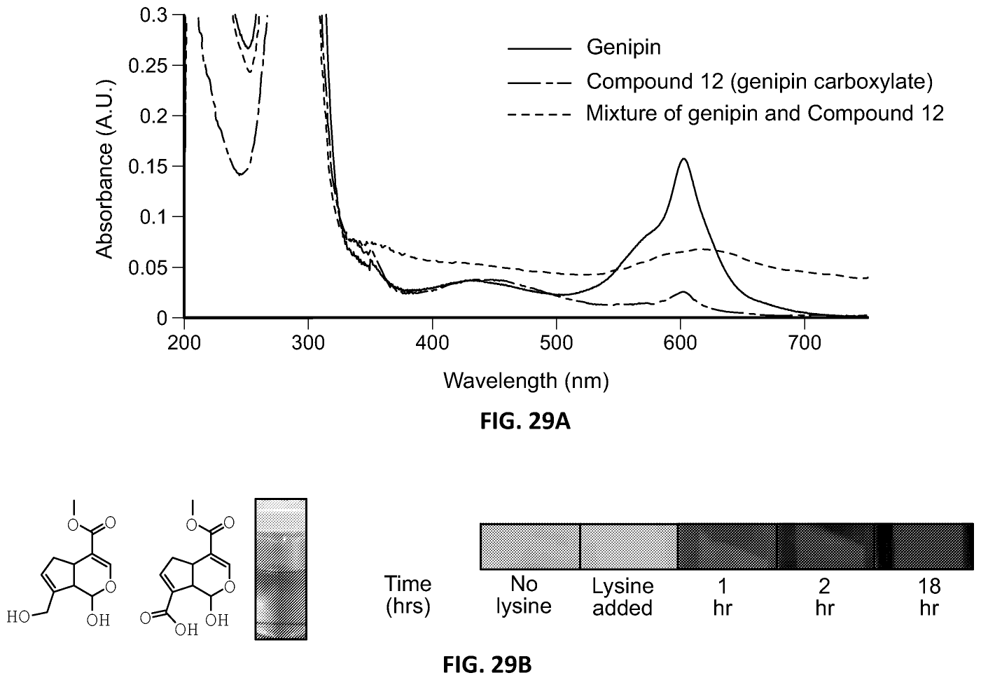

Genipin and Compound 12 were weighed out (n=0.03 mmol each) into a small vial equipped with a magnetic stir bar. To this, 1.5 mL of methanol was added followed by L-lysine (0.12 mmol, 2 equivalents). The reaction mixture was stirred for 24 hours at room temperature under open air. This mixture was diluted with methanol to make 0.06 mM solutions for UV-VIS characterization. See FIGS. 29A and 29B. See also FIGS. 32 and 33.

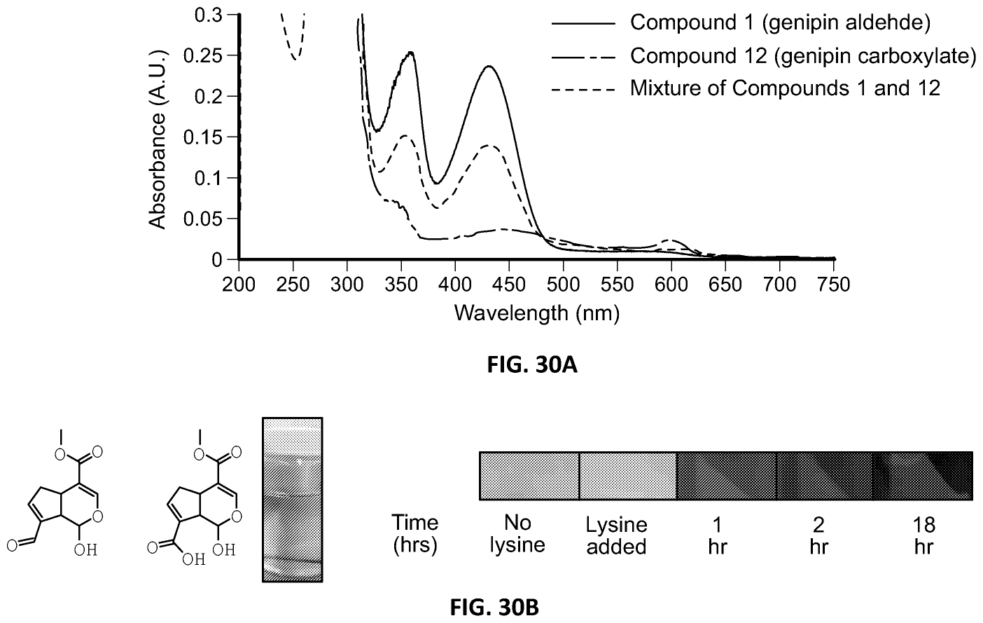

Compound 1 and Compound 12 were weighed out (n=0.03 mmol each) into a small vial equipped with a magnetic stir bar. To this, 1.5 mL of methanol was added followed by L-lysine (0.12 mmol, 2 equivalents). The reaction mixture was stirred for 24 hours at room temperature under open air. This mixture was diluted with methanol to make 0.06 mM solutions for UV-VIS characterization. See FIGS. 30A and See also FIGS. 32 and 33.

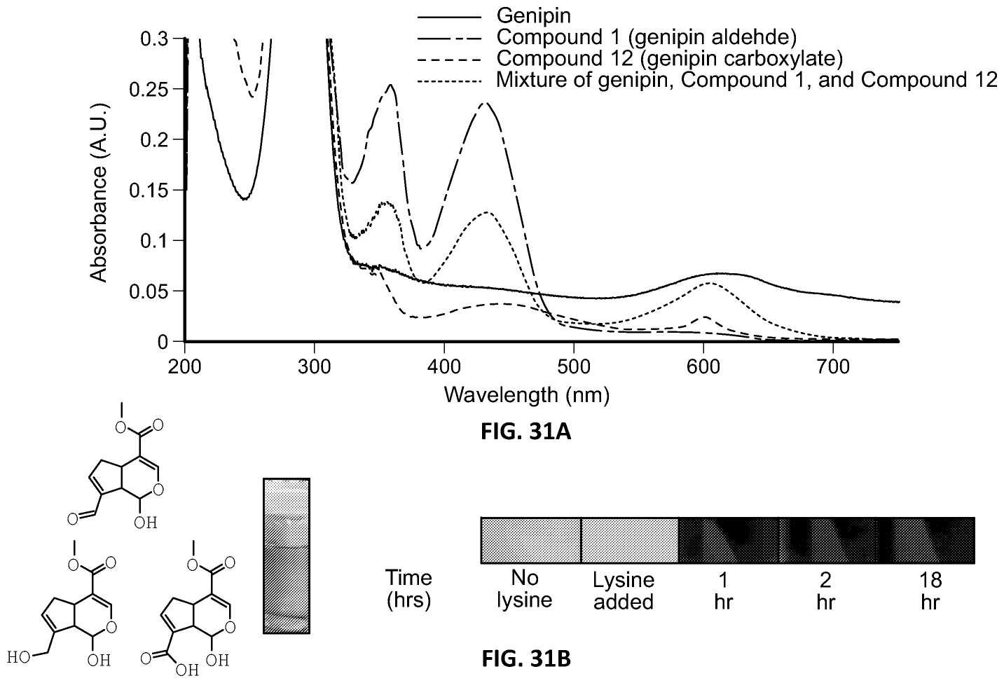

Genipin, Compound 1 and Compound 12 were weighed out (n=0.03 mmol each) into a small vial equipped with a magnetic stir bar. To this, 1.5 mL of methanol was added followed by L-lysine (0.18 mmol, 2 equivalents). The reaction mixture was stirred for 24 hours at room temperature under open air. This mixture was diluted with methanol to make 0.06 mM solutions for UV-VIS characterization. See FIGS. 31A and 31B. See also FIGS. 32 and 33.

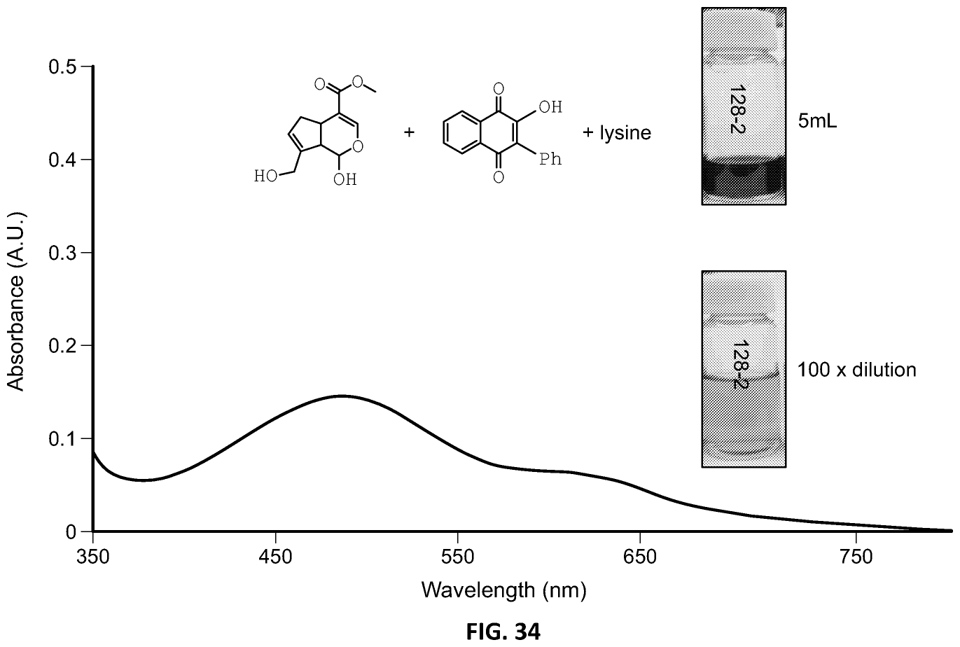

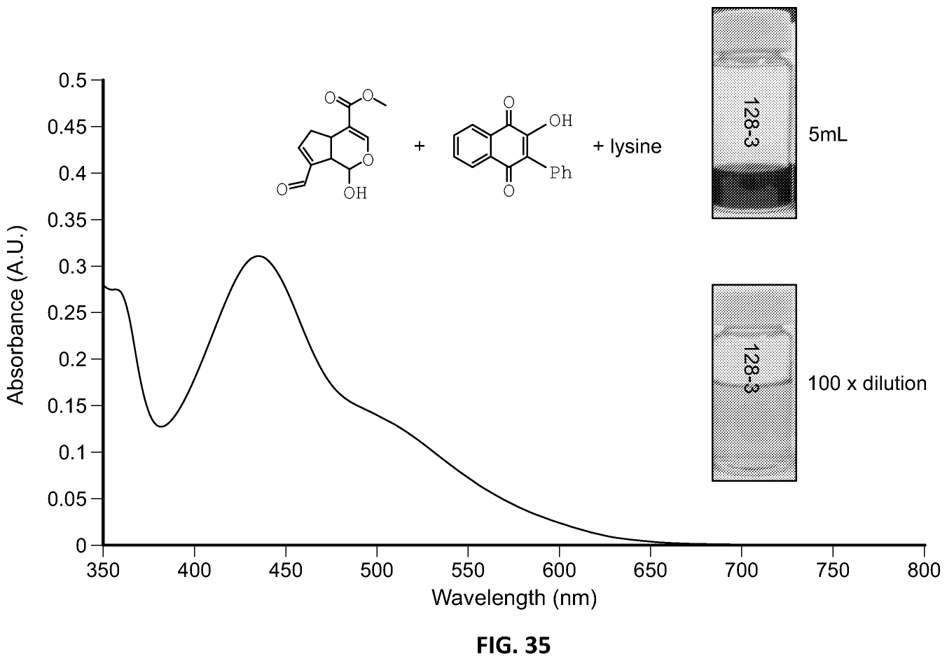

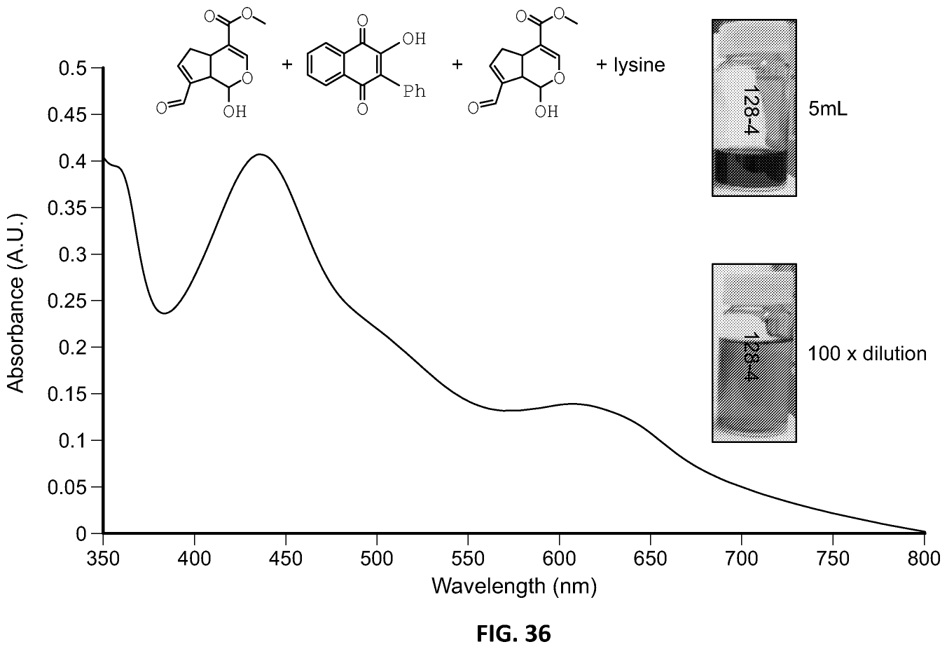

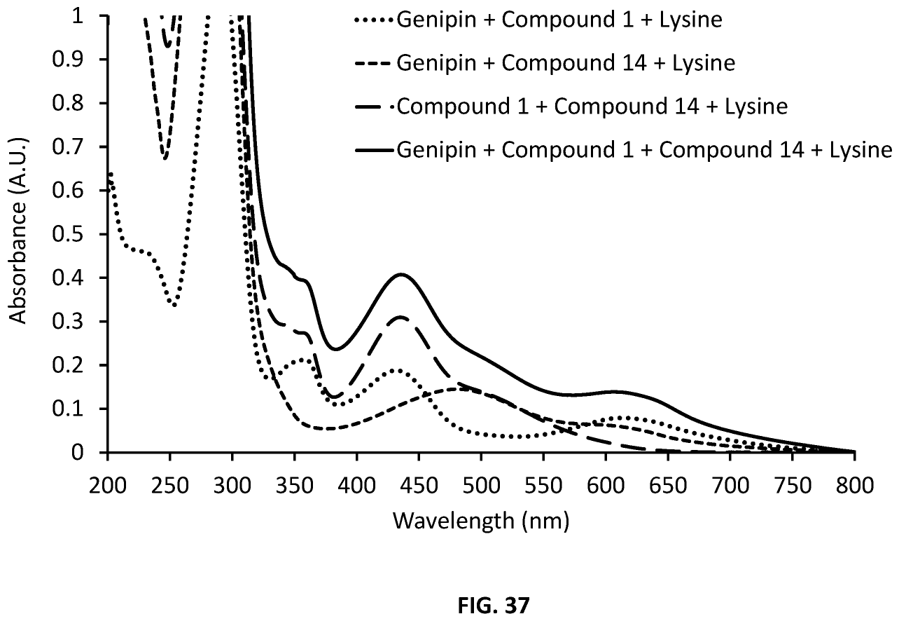

FIGS. 34-37 shows the color profile for compositions containing genipin, lawsone, Compound 1, and Compound 14 including single, binary, and ternary dye combinations. Table 5 shows the color profile for compositions containing genipin and Compounds 1, 2, and 14 including single, binary, and ternary dye combinations.

TABLE 5

| | Conc. | | | |
|---|---|---|---|---|
| | (mmol/g) of | | Color when | |
| Compounds | total dye in the | | reacted | |
| Present | final | | with amino | |
| (mole ratio) | formulation | Color | acids | Solubility |
| Genipin, | 0.008-0.400 | Green | Bright Forest | Similar in |
| Compound 1 | | | Green | ethanol and |
| (1:1) | | | | water |
| Compound 1, | 0.008-0.401 | Orange | Bright | Similar in |
| Compound 14 | | | Orange | ethanol and |
| (1:1) | | | | water |
| Genipin, | 0.008-0.402 | Purple | Vibrant | Similar in |
| Compound 14 | | | Purple | ethanol and |
| (1:1) | | | | water |
| Genipin, | 0.008-0.403 | Black/Brown | Dark | Similar in |
| Compound 1, | | | Black/Brown | ethanol and |
| Compound 14 | | | | water |
| (1:1:1) | | | | |
| Compound 1 | 0.008-0.404 | N/A | Vibrant | Similar in |
| | | | Yellow | ethanol and |
| | | | | water |
| Compound 2 | 0.008-0.405 | N/A | Vibrant Red | Similar in |
| | | | | ethanol and |
| | | | | water |

Additional Color Combinations

Color mixing of the compounds described herein can be utilized to yield different colors and shades. These could be binary, ternary, quaternary, etc. mixtures with varying percentages of each.

Blue: the active colorant in these tattoos includes genipin.

Yellow: Compound 1 produces a vibrant yellow color upon reaction with amino acids.

Red: Compounds 11, 7, and 9 produce a vibrant red color upon reaction with amino acids.

Green: Compound 12 produces a vibrant green color upon reaction with amino acids.

Orange: Compound 13 produces a vibrant orange color upon reaction with amino acids.

### OTHER EMBODIMENTS

It is to be understood that while the disclosure has been described in conjunction with the detailed description thereof, the foregoing description is intended to illustrate and not limit the scope of the disclosure which is defined by the scope of the appended claims. Other aspects, advantages, and modification are within the scope of the following claims.

1.-239. (canceled)

240. A composition comprising:
a semi-permanent colorant, wherein the semi-permanent colorant comprises a genipin derivative;
a solvent; and
further comprising a semi-volatile semi-permanent colorant solubilizer or a film-forming agent or a thickening agent;
and wherein the genipin derivative is a compound of Formula I: 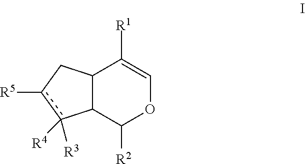
wherein:
R¹ is —C(═O)OR⁶, C₁₋₆ alkyl, or C₆₋₁₀ aryl, wherein said C₁₋₆ alkyl and C₆₋₁₀ aryl are each optionally substituted with 1-5 R⁷;
R⁶ is H, C₁₋₆ alkyl, or C₆₋₁₀ aryl, wherein said C₁₋₆ alkyl and C₆₋₁₀ aryl are each optionally substituted with 1-5 R⁷;
each R⁷ is independently an electron withdrawing or an electron donating group;
R² is H, —OR⁹, or OC(═O)R^{9A};
R⁹ is selected from the group consisting of: H, C₁₋₆ alkyl, C₆₋₁₀ aryl, and a hydroxyl-protecting group;
R^{9A} is selected from C₁₋₆ alkyl and C₆₋₁₀ aryl; and
the dotted line indicates the optional presence of a double bond;
wherein if the double bond is absent, then R⁴ and R⁵ together with the carbon atoms to which they are attached form a 3-4 membered heterocyclic ring and R³ is —OH or —CH₂OH; or
if the double bond is present, then R⁴ and R⁵ are absent and R³ is selected from the group consisting of: —NR⁷R⁸, —CH₂NR⁷R⁸, —C(═O)OR¹⁰, —C(═O)NR¹⁰R¹⁰, —CH(═O), phenyl, —CHR¹⁰R¹⁰, —CH═CHR¹⁰, —CH═CR¹⁰R¹⁰, —CH₂OR¹⁰, and phenyl substituted with 1-5 R⁷;
wherein R¹⁰ is selected from H, C₁₋₆ alkyl, alkoxy, oxo, —OH, —CH(═O), —C(═O)OR⁶, phenyl, and phenyl substituted with 1-5 R⁷; and
R⁸ is H or R⁷.

241. The composition of claim 240, wherein the semi-permanent colorant further comprises genipin.

242. The composition of claim 240, wherein:
R¹ is —C(═O)OR⁶ or phenyl substituted with 1-5 R⁷;
R⁶ is H, C₁₋₆ alkyl, or phenyl substituted with 1-5 R⁷;
R² is H, —OR⁹, or OC(═O)R⁹;
R⁹ is H or C₁₋₆ alkyl;
if the double bond is present, then R⁴ and R⁵ are absent and R³ is selected from the group consisting of: —NR⁷R⁸, —CH₂NR⁷R⁸, —C(═O)OR¹⁰, —CH(═O), phenyl, —CHR¹⁰R¹⁰, —CH═CHR¹⁰, —CH₂OR¹⁰, diethyl malonate, and phenyl substituted with 1-5 R⁷; and
R¹⁰ is selected from H, C₁₋₆ alkyl, alkoxy, oxo, —OH, —CH(═O) and phenyl substituted with 1-5 R⁷.

243. The composition of claim 240,
wherein R¹ is —C(═O)OR⁶ and R⁶ is methyl; or
wherein R¹ is —C(═O)OR⁶ and wherein R⁶ is phenyl substituted with 1-5 R⁷.

244. The composition of claim 240,
wherein R² is —OC(═O)CH₃; or
wherein R² is —OH; or
wherein R² is —OR⁹ and R⁹ is selected from the group consisting of: H, C₁₋₆ alkyl, C₆₋₁₀ aryl, and a tri(C₁₋₆ alkyl)silyl.

245. The composition of claim 240, wherein R⁹ is selected from the group consisting of: H, C₁₋₆ alkyl, C₆₋₁₀ aryl, and a tri(C₁₋₆ alkyl)silyl.

246. The composition of claim 240, wherein the double bond is absent.

247. The composition of claim 246, wherein then R⁴ and R⁵ together with the carbon atoms to which they are attached form a 3-membered heterocyclic ring and R³ is —CH₂OH; or wherein then R⁴ and R⁵ together with the carbon atoms to which they are attached form a 4-membered heterocyclic ring and R³ is —OH.

248. The composition of claim 240, wherein the double bond is present.

249. The composition of claim 248, wherein R³ is selected from the group consisting of: —NR⁷R⁸, —CH₂NR⁷R⁸, —C(═O)OR¹⁰, —C(═O)N R¹⁰R¹⁰, —CH(═O), phenyl, —CHR¹⁰R¹⁰, —CH═CHR¹⁰, —CH═CR¹⁰R¹⁰, —CH₂OR¹⁰, and phenyl substituted with 1-5 R⁷;
R¹ is —C(═O)OH;
R² is —OH; and
R¹⁰ is selected from H, C₁₋₆ alkyl, alkoxy, oxo, —OH, —CH(═O), —C(═O)OR⁶, phenyl, and phenyl substituted with 1-5 R⁷,
and/or
wherein R³ is selected from the group consisting of: —NR⁷R⁸, —CH₂—NR⁷R⁸, —C(═O)OR¹⁰, —CH(═O), phenyl, —CHR¹⁰R¹⁰, —CH═CHR¹⁰, —C(═O)NR¹⁰R¹⁰, —CH₂OR¹⁶, and phenyl substituted with 1-5 R⁷;
R¹ is —C(═O)OH; and
R² is —OR⁹ and R⁹ is selected from the group consisting of: H, C₁₋₆ alkyl, tert-butyldimethylsilyl (TBS), trimethylsilyl (TMS), triethylsilyl (TES), and C₆₋₁₀ aryl,
and/or
wherein R³ is selected from the group consisting of: —NR⁷R⁸, —CH₂—NR⁷R⁸, —C(═O)OR¹⁰, —CH(═O), phenyl, —CHR¹⁰R¹⁰, —CH═CHR¹⁰, —CH₂OR¹⁰, diethyl malonate, and phenyl substituted with 1-5 R⁷;
R¹ is —C(═O)OH; and
R² is —OH.

250. The composition of claim 240, wherein the genipin derivative is a compound selected from the group consisting of the compounds delineated in Table 1.

251. The composition of claim 1, wherein R⁷ is selected from the group consisting of: —(C₁₋₉ alkyl), —(C₂₋₉ alkenyl), phenyl, —NR⁸R⁸, —OR⁸, —NHC(═O)R⁸, —OC(═O)R⁸, —SR⁸, —CO₂⁻, —SO₂CF₃, —NO, —NO₂, —SO₂R⁸, —CN, —CR¹¹, —COR¹¹, —CHO, —CO₂R⁸, —C(═O)NR⁸R⁸, and halide;
wherein R⁸ is selected from the group consisting of: H, —(C₁₋₉ alkyl), and —(C₂₋₉ alkenyl); and R¹¹ is halide.

252. The composition of claim 240, wherein the semi-permanent colorant is a combination of a genipin derivative with one or more compounds chosen from genipin, lawsone and a further genipin derivative.

253. The composition of claim 240, wherein the composition comprises semi-volatile semi-permanent colorant solubilizer and further comprises a surface-active agent, and/or a matrix component, and/or a skin penetration enhancer, and/or a temporary colorant, and/or a preservative.

254. The composition of claim 240, wherein the composition comprises a thickening agent and further comprises a film-forming agent, and/or a humectant and/or a preservative; or
wherein the composition comprises a film-forming agent and further comprises a humectant and/or a preservative.

255. A compound of Formula IIa: 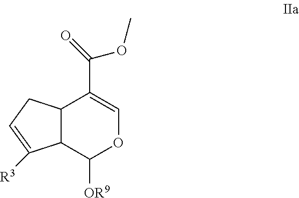
or a pharmaceutically acceptable salt thereof, wherein:
R⁹ is selected from the group consisting of: H, C₁₋₆ alkyl, C₆₋₁₀ aryl, and a hydroxyl-protecting group, wherein said C₁₋₆ alkyl and C₆₋₁₀ aryl are each optionally substituted with 1-5 R⁷;
R³ is selected from the group consisting of: —CH₂NR⁷R⁸, —C(═O)OR¹⁰, —C(═O)N R¹⁰R¹⁰, —CH(═O), —CHR¹⁰R¹⁰, —CH═CHR¹⁰, —CH═CR¹⁰R¹⁰, —CH₂OR¹⁰, phenyl, and phenyl substituted with 1-5 R⁷;
wherein each R¹⁰ is independently selected from H, C₁₋₆ alkyl, —OH, —CH(═O), C(═O)OR⁹, phenyl, and phenyl substituted with 1-5 R⁷; and
R⁷ is an electron withdrawing or an electron donating group; and
R⁸ is H or R⁷.

256. The compound of claim 255, wherein:
R³ is selected from —C(═O)OR¹⁰, —CH(═O), and —CH₂OR¹⁰;
R¹⁰ is selected from H, C₁₋₆ alkyl, phenyl, and phenyl substituted with 1-5 R⁷; and
R⁹ is selected from C₁₋₆ alkyl, C₆₋₁₀ aryl, and a hydroxyl-protecting group, wherein said C₁₋₆ alkyl and C₆₋₁₀ aryl are each optionally substituted with 1-5 R⁷,
or wherein:
R³ is —CH═CR¹⁰R¹⁰;
each R¹⁰ is independently CH(═O) or C(═O)OR⁹; and
R⁹ is selected from C₁₋₆ alkyl, C₆₋₁₀ aryl, and a hydroxyl-protecting group, wherein said C₁₋₆ alkyl and C₆₋₁₀ aryl are each optionally substituted with 1-5 R⁷, or wherein:
R³ is selected from —CH₂NR⁷R⁸, —C(═O)N R¹⁰R¹⁰, —CH(OH)R¹⁰, —CH═CHR¹⁰, phenyl, and phenyl substituted with 1-5 R⁷; and
each R¹⁰ is independently selected from C₁₋₆ alkyl, phenyl, and phenyl substituted with 1-5 R⁷.

257. The compound of claim 255, wherein the compound has formula IIb: 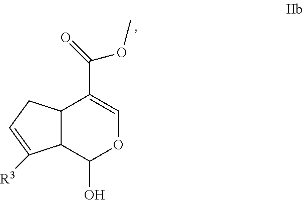
or a pharmaceutically acceptable salt thereof.

258. The compound of claim 255, wherein the compound is selected from the group consisting of the compounds delineated in Table 2 or Table 3.

259. A compound selected from the group consisting of: 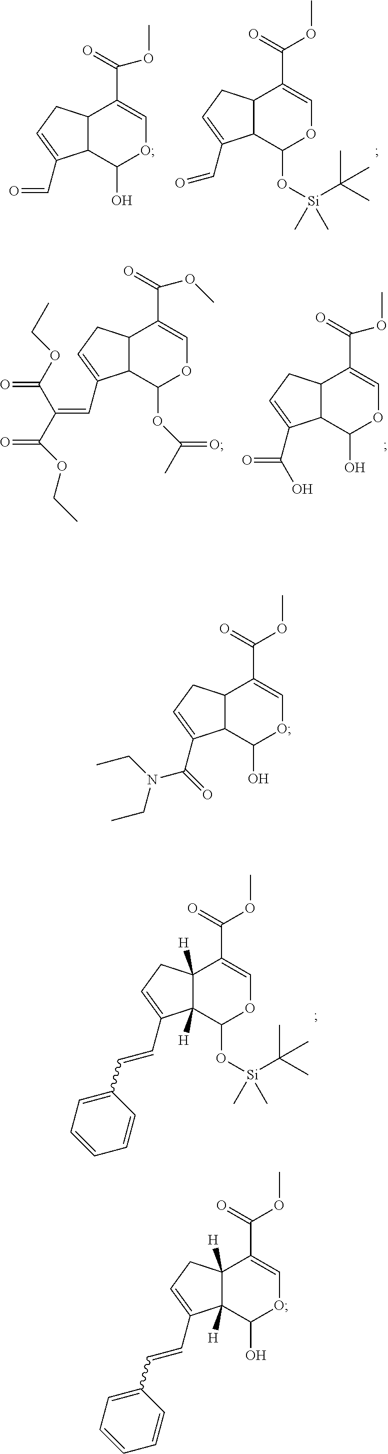 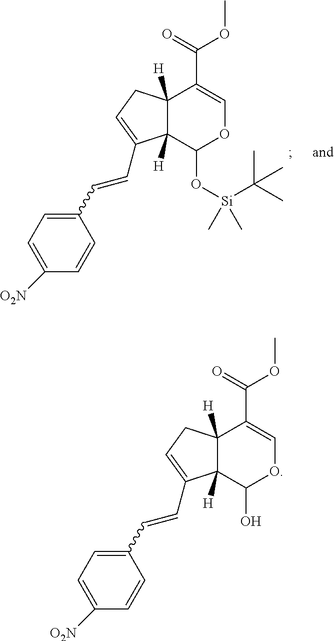 [SMILES: COC(=O)C1=COC(O[Si](C)(C)C(C)(C)C)[C@@H]2C(C=Cc3ccc([N+](=O)[O-])cc3)=CC[C@H]12 ; COC(=O)C1=COC(O)[C@@H]2C(C=Cc3ccc([N+](=O)[O-])cc3)=CC[C@H]12]
